(19) United States
(12) Patent Application Publication
(10) Pub. No.: US 2011/0189668 A1
(43) Pub. Date: Aug. 4, 2011

(54) Methods for determining likelihood of longevity and of developing an age-related disease

(76) Inventors: Aviv Bergman, New York, NY (US); Nir Barzilai, Hartsdale, NY (US); Gil Atzmon, New York, NY (US)

(21) Appl. No.: 12/930,972

(22) Filed: Jan. 21, 2011

Related U.S. Application Data
(60) Provisional application No. 61/336,987, filed on Jan. 29, 2010.

(51) Int. Cl. C12Q 1/68

(52) U.S. Cl. 435/6.11

(57) ABSTRACT

The present invention provides a method for identifying a subject's likelihood of achieving longevity or of developing an age-related disease, the method for identifying a subject's likelihood of achieving longevity or of developing an age-related disease comprising detecting at least one specified single nucleotide polymorphisms or genotype at a single nucleotide polymorphism or combination of a genotype at a first single nucleotide polymorphism and a genotype at a second single nucleotide polymorphism in a sample of the subject's DNA and, wherein the detection of at least one single nucleotide polymorphisms or genotype at a single nucleotide polymorphism or combination of a genotype at a first single nucleotide polymorphism and a genotype at a second single nucleotide polymorphism identifies the subject's as likely achieving longevity or of developing an age-related disease.

### CROSS-REFERENCE TO RELATED APPLICATIONS

This application claims benefit of U.S. Provisional Application No. 61/336,987, filed Jan. 29, 2010, the contents of which are hereby incorporated by reference.

### STATEMENT OF GOVERNMENT SUPPORT

This invention was made with government support under grant numbers AG028872 and AG027734 awarded by the National Institute on Aging, National Institutes of Health, U.S. Department of Health and Human Services. The government has certain rights in the invention.

### FIELD OF THE INVENTION

The present invention relates to the use of specific single nucleotide polymorphisms (SNPs) for determining an individual's likelihood of longevity and/or of developing age-related disease.

### BACKGROUND OF THE INVENTION

Throughout this application various publications are referred to in brackets. Full citations for these references may be found at the end of the specification. The disclosures of these publications are hereby incorporated by reference in their entirety into the subject application to more fully describe the art to which the subject invention pertains.

Defining the genetic and environmental factors that influence longevity in humans has profound implications for the development of strategies to delay or prevent age-related diseases. Identification of specific traits relevant to aging phenotypes and genes that promote longevity provide important mechanistic insights into the molecular basis of aging.

Subjects with exceptional longevity are generally spared major age-related diseases, such as cardiovascular disease, diabetes mellitus, Alzheimer's disease and cancer, which are responsible for most deaths in the elderly [1]. Studies suggest that while the effect of genetics on life expectancy is minimal across ages, this is not the case with centenarians (a phenotype achieved by only ˜ 1/10,000 individuals). Siblings of current centenarians have odds ratios of 8-17 of achieving 100 years of age, and parents of centenarians (born at ˜1870) had odds ratio of ˜7 of achieving ages 90-99 than an appropriate control [2-4]. Furthermore, offspring of long-lived parents had significantly lower prevalence (˜50%) of hypertension, diabetes mellitus, myocardial infarctions and strokes/transient ischemic attacks compared with several age-matched control groups [4-5]. In support of the inheritance of longevity, the New England Centenarian Study reported statistically significant linkage between a genetic locus on chromosome 4 and exceptional longevity among siblings [3, 6].

While life-style factors such as obesity have been identified as limiting factors in life expectancy [7], genetic factors are considered central to the process of aging. However, the long and complex list of biological processes associated with aging has not permitted all causes of aging to be determined. Although monogenetic disorders such as abnormalities in the LDL receptor gene in cardiovascular disease [8, 9] and the BRCA1 and BRCA2 genes in breast cancer [10] represent genetically determined phenotypic defects associated with early mortality, they are not common mechanisms of aging. The inherited biological factors determining lifespan in long-lived humans remains unknown [11].

Despite the advances in knowledge of treating age-related illnesses such as dementia, metabolic- and cardiovascular-related diseases, there remains a clear need for markers of longevity which may be used to decrease the risk of developing such age-related diseases, which may be used to advance treatment for such diseases, and/or which can permit for early treatment of such diseases.

### SUMMARY OF THE INVENTION

A method for identifying a subject's likelihood of achieving longevity comprising detecting at least one of the following single nucleotide polymorphisms in a sample of a subject's DNA: nucleotide A at rs2410082, nucleotide A at rs11094258, nucleotide C at rs12131971, nucleotide C at rs966272, nucleotide G at rs6851158, nucleotide G at rs12556351, nucleotide G at rs5991963, nucleotide T at rs1047972, or nucleotide T at rs11001056,

wherein detection of the presence of one of more of the single nucleotide polymorphisms identifies the subject as likely to achieve of longevity.

A method for identifying a subject's likelihood of developing an age-related disease comprising detecting at least one of the following single nucleotide polymorphisms in a sample of a subject's DNA: nucleotide A at rs16951040, nucleotide A at rs17625605, nucleotide A at rs3743180, nucleotide A at rs325380, nucleotide A at rs1528061, nucleotide C at rs17095079, nucleotide A at rs10775623, nucleotide A at rs10515725, nucleotide C at rs2061007, nucleotide A at rs4479454, nucleotide C at rs7630086, nucleotide C at rs3865868, nucleotide C at rs16987522, nucleotide C at rs16916036, nucleotide A at rs11818525, nucleotide A at rs17696856, nucleotide A at rs17014445, nucleotide T at rs1808723, nucleotide T at rs17049044, nucleotide G at rs11497898, nucleotide G at rs16916055, nucleotide T at rs2274703, nucleotide T at rs7067297, nucleotide G at rs2525841, nucleotide G at rs5951437, nucleotide T at rs1991921, nucleotide T at rs11247124, nucleotide T at rs3848883, or nucleotide G at rs5981402, wherein detection of the presence of one or more of the single nucleotide polymorphisms in the subject's DNA identifies the subject as likely to develop an age-related disease.

A method for identifying a subject's likelihood of achieving longevity comprising detecting at least one of the following genotypes in a sample of a subject's DNA at a single nucleotide polymorphism: genotype CC at rs6470902, genotype GC at rs6470902, genotype BB at rs7168796, genotype BB at rs16853867, genotype AA at rs17049044, genotype AA at rs11020277, genotype AA at rs11020294, genotype AA at rs7653267, genotype AA at rs11909021, genotype AB at rs12010050, genotype AB at rs9488847, genotype AB at rs1255575, genotype AB at rs16895959, genotype AB at rs716682, genotype BB at rs2289507, genotype BB at rs17043069, or genotype BB at rs7038833,

wherein the presence of one or more of the genotypes at a single nucleotide polymorphism increases the subject's likelihood of longevity.

A method for identifying a subject's likelihood of developing an age-related disease comprising detecting at least one of the following genotypes at a single nucleotide polymorphism in a sample of the subject's DNA: genotype CC at rs9914076, genotype CC at rs16971905, genotype CC at rs7272951, genotype CC at rs6093591, genotype CG at rs509193, genotype AA at rs11164749, genotype AA at rs1712676, genotype AA at rs12521591, genotype AA at rs7168796, genotype AA at rs16853867, genotype AB at rs16929656, genotype AB at rs3814284, genotype AB at rs6009526, genotype AB at rs7780235, genotype AB at rs2206100, genotype BB at rs16895959, genotype BB at rs716682, genotype BB at rs702743, genotype BB at rs13355963, or genotype BB at rs16860657, wherein the presence of one or more of the genotypes at a single nucleotide polymorphism increases the subject's likelihood of developing an age-related disease.

A method for identifying a subject's likelihood of achieving longevity comprising detecting in a sample of the subject's DNA at least one of the following combinations of a genotype at a single nucleotide polymorphism from column 1 and a corresponding genotype at a single nucleotide polymorphism from column 2:

| | Column 1 | | Column 2 | |
|---|---|---|---|---|
| | Genotype | dbSNP | Genotype | dbSNP |
| 1 | CG | rs10041500 | AG | rs5912192 |
| 2 | CC | rs10738555 | GG | rs10919593 |
| 3 | GC | rs10738555 | GG | rs10919593 |
| 4 | TT | rs10919593 | CT | rs642053 |
| 5 | AT | rs10919593 | CT | rs642053 |
| 6 | CT | rs11738547 | CC | rs11030746 |
| 7 | CT | rs11738547 | TT | rs11030746 |
| 8 | CT | rs11738547 | AA | rs1257689 |
| 9 | CT | rs11738547 | GG | rs17120428 |
| 10 | CT | rs11738547 | AA | rs3749796 |
| 11 | CT | rs11738547 | TT | rs3749796 |
| 12 | TT | rs12276840 | AC | rs10227794 |
| 13 | TT | rs12276840 | CC | rs10227794 |
| 14 | TT | rs12276840 | CC | rs2274703 |
| 15 | CC | rs12276840 | CC | rs2274703 |
| 1 | CC | rs12348066 | GG | rs496666 |
| 17 | CG | rs12348066 | GG | rs496666 |
| 18 | CC | rs12348066 | CC | rs536789 |
| 19 | CG | rs12348066 | CC | rs536789 |
| 20 | CC | rs12348066 | GG | rs5912192 |
| 21 | CG | rs12348066 | GG | rs5912192 |
| 22 | CC | rs12348066 | TT | rs7063827 |
| 23 | CG | rs12348066 | TT | rs7063827 |
| 24 | CC | rs12432648 | CT | rs12838423 |
| 25 | GG | rs12432648 | CT | rs12838423 |
| 26 | CC | rs12432648 | GT | rs12857956 |
| 27 | GG | rs12432648 | GT | rs12857956 |
| 28 | AG | rs1255575 | CC | rs2862274 |
| 29 | AG | rs1255575 | TT | rs2862274 |
| 30 | CC | rs1255575 | CC | rs2862274 |
| 31 | CG | rs1255575 | CC | rs2862274 |
| 32 | CC | rs1255575 | TT | rs2862274 |
| 33 | CG | rs1255575 | TT | rs2862274 |
| 34 | GG | rs1430707 | TT | rs2274703 |
| 35 | GG | rs1430707 | CC | rs2862274 |
| 36 | GG | rs1430707 | TT | rs2862274 |
| 37 | GG | rs1430707 | AA | rs3749796 |
| 38 | GG | rs1461809 | TT | rs3816612 |
| 39 | CC | rs1499953 | AA | rs10919593 |
| 40 | AG | rs1528708 | GG | rs4595891 |
| 41 | TT | rs16880188 | CC | rs11030746 |
| 42 | AT | rs16880188 | CC | rs11030746 |
| 43 | TT | rs16880188 | TT | rs11030746 |
| 44 | AT | rs16880188 | TT | rs11030746 |
| 45 | TT | rs16880188 | CC | rs17003277 |
| 46 | AT | rs16880188 | CC | rs17003277 |
| 47 | TT | rs16880188 | TT | rs17003277 |
| 48 | AT | rs16880188 | TT | rs17003277 |
| 49 | TT | rs16951040 | CC | rs1324362 |
| 50 | AT | rs16951040 | CC | rs1324362 |
| 51 | CC | rs16951040 | AG | rs17154320 |
| 52 | TT | rs17014445 | GG | rs10919593 |
| 53 | AT | rs17014445 | GG | rs10919593 |
| 54 | TT | rs17014445 | GG | rs10919593 |
| 55 | GG | rs17014445 | AA | rs10919593 |
| 56 | GG | rs17014445 | GG | rs10919593 |
| 57 | GG | rs17014445 | AA | rs10919593 |
| 58 | TT | rs17014445 | CC | rs11103184 |
| 59 | AT | rs17014445 | CC | rs11103184 |
| 60 | GG | rs17014445 | CC | rs11103184 |
| 61 | TT | rs17014445 | CC | rs11103512 |
| 62 | AT | rs17014445 | CC | rs11103512 |
| 63 | GG | rs17014445 | CC | rs11103512 |
| 64 | TT | rs17014445 | CC | rs11183915 |
| 65 | AT | rs17014445 | CC | rs11183915 |
| 66 | GG | rs17014445 | CC | rs11183915 |
| 67 | GG | rs17014445 | AG | rs17154320 |
| 68 | TT | rs17014445 | TT | rs1822910 |
| 69 | AT | rs17014445 | TT | rs1822910 |
| 70 | GG | rs17014445 | TT | rs1822910 |
| 71 | GG | rs17014445 | AA | rs2525841 |
| 72 | GG | rs17014445 | AA | rs3749796 |
| 73 | GG | rs17014445 | AA | rs3925518 |
| 74 | GG | rs17014445 | TT | rs6077004 |
| 75 | GG | rs17014445 | CT | rs6419449 |
| 76 | GG | rs17014445 | CG | rs6540375 |
| 77 | GG | rs17014445 | AT | rs6641231 |
| 78 | GG | rs17014445 | CC | rs7827228 |
| 79 | TT | rs17014445 | CT | rs9586459 |
| 80 | AT | rs17014445 | CT | rs9586459 |
| 81 | TT | rs17014445 | TT | rs9586459 |
| 82 | AT | rs17014445 | TT | rs9586459 |
| 83 | GG | rs17014445 | CT | rs9586459 |
| 84 | GG | rs17014445 | TT | rs9586459 |
| 85 | AA | rs17049044 | CT | rs3859965 |
| 86 | AT | rs17049044 | CT | rs3859965 |
| 87 | CC | rs17049044 | AA | rs553369 |
| 88 | AA | rs17083201 | GT | rs2144497 |
| 89 | AT | rs17083201 | GT | rs2144497 |
| 90 | AA | rs17154320 | CC | rs10278652 |
| 91 | GG | rs17154320 | CC | rs10278652 |
| 92 | AA | rs17372695 | CC | rs2862274 |
| 93 | AT | rs17372695 | CC | rs2862274 |
| 94 | AA | rs17372695 | TT | rs2862274 |
| 95 | AT | rs17372695 | TT | rs2862274 |
| 96 | CT | rs17775764 | AG | rs5912192 |
| 97 | TT | rs1822910 | AC | rs11625236 |
| 98 | TT | rs1822910 | CC | rs2274703 |
| 99 | TT | rs1822910 | CT | rs2613216 |
| 100 | TT | rs1822910 | TT | rs2613216 |
| 101 | TT | rs1822910 | AA | rs2623149 |
| 102 | TT | rs1822910 | AG | rs2623149 |
| 103 | TT | rs1822910 | GG | rs5981167 |
| 104 | AT | rs1822910 | GG | rs5981167 |
| 105 | AA | rs1991921 | CC | rs11030746 |
| 106 | AT | rs1991921 | CC | rs11030746 |
| 107 | AA | rs1991921 | TT | rs11030746 |
| 108 | AT | rs1991921 | TT | rs11030746 |
| 109 | AA | rs1991921 | TT | rs3749796 |
| 110 | AT | rs1991921 | TT | rs3749796 |
| 111 | AA | rs1991921 | CC | rs3816612 |
| 112 | AT | rs1991921 | CC | rs3816612 |
| 113 | GG | rs2012129 | CC | rs11030746 |
| 114 | CG | rs2012129 | CC | rs11030746 |
| 115 | GG | rs2012129 | TT | rs11030746 |
| 116 | CG | rs2012129 | TT | rs11030746 |
| 117 | GG | rs2012129 | TT | rs3749796 |
| 118 | CG | rs2012129 | TT | rs3749796 |
| 119 | GG | rs2061007 | CC | rs11030746 |
| 120 | CG | rs2061007 | CC | rs11030746 |
| 121 | GG | rs2061007 | TT | rs11030746 |
| 122 | CG | rs2061007 | TT | rs11030746 |
| 123 | GG | rs2061007 | AA | rs16902320 |
| 124 | CG | rs2061007 | AA | rs16902320 |
| 125 | GG | rs2061007 | CC | rs16902320 |
| 126 | CG | rs2061007 | CC | rs16902320 |
| 127 | GG | rs2061007 | CC | rs17003277 |
| 128 | CG | rs2061007 | CC | rs17003277 |
| 129 | GG | rs2061007 | TT | rs1822910 |
| 130 | CG | rs2061007 | TT | rs1822910 |
| 131 | GG | rs2061007 | TT | rs2274703 |
| 132 | CG | rs2061007 | TT | rs2274703 |
| 133 | GG | rs2061007 | CC | rs2862274 |
| 134 | CG | rs2061007 | CC | rs2862274 |
| 135 | GG | rs2061007 | TT | rs2862274 |
| 136 | CG | rs2061007 | TT | rs2862274 |
| 137 | GG | rs2061007 | TT | rs3749796 |
| 138 | CG | rs2061007 | TT | rs3749796 |
| 139 | GG | rs2061007 | CC | rs3816612 |
| 140 | CG | rs2061007 | CC | rs3816612 |
| 141 | GG | rs2061007 | TT | rs3816612 |
| 142 | CG | rs2061007 | TT | rs3816612 |
| 143 | GG | rs2098846 | CC | rs17003277 |
| 144 | CG | rs2098846 | CC | rs17003277 |
| 145 | GG | rs2098846 | TT | rs17003277 |
| 146 | CG | rs2098846 | TT | rs17003277 |
| 147 | GG | rs2098846 | TT | rs2274703 |
| 148 | CG | rs2098846 | TT | rs2274703 |
| 149 | CC | rs2274703 | CG | rs2237342 |
| 150 | AA | rs2274703 | CG | rs2237342 |
| 151 | AT | rs2274703 | CG | rs2237342 |
| 152 | CC | rs2274703 | CC | rs7827228 |
| 153 | AG | rs2382993 | CC | rs11103184 |
| 154 | AA | rs2525841 | CT | rs12838423 |
| 155 | AA | rs2525841 | GT | rs12857956 |
| 156 | CC | rs2525841 | AG | rs5981167 |
| 157 | CG | rs2525841 | AG | rs5981167 |
| 158 | CC | rs2525841 | GG | rs5981167 |
| 159 | CG | rs2525841 | GG | rs5981167 |
| 160 | TT | rs2615903 | AG | rs17154320 |
| 161 | TT | rs2615903 | AA | rs2525841 |
| 162 | TT | rs2615903 | AA | rs3749796 |
| 163 | AA | rs2762933 | CC | rs2862274 |
| 164 | AT | rs2762933 | CC | rs2862274 |
| 165 | AA | rs2762933 | TT | rs2862274 |
| 166 | AT | rs2762933 | TT | rs2862274 |
| 167 | TT | rs325380 | CC | rs11030746 |
| 168 | AT | rs325380 | CC | rs11030746 |
| 169 | TT | rs325380 | TT | rs11030746 |
| 170 | AT | rs325380 | TT | rs11030746 |
| 171 | TT | rs325380 | TT | rs3749796 |
| 172 | AT | rs325380 | TT | rs3749796 |
| 173 | CC | rs368866 | CC | rs17003277 |
| 174 | CG | rs368866 | CC | rs17003277 |
| 175 | CC | rs368866 | TT | rs17003277 |
| 176 | CG | rs368866 | TT | rs17003277 |
| 177 | TT | rs4686457 | AA | rs10919593 |
| 178 | TT | rs4686457 | TT | rs11030746 |
| 179 | TT | rs4686457 | AA | rs16902320 |
| 180 | TT | rs4686457 | CC | rs16902320 |
| 181 | TT | rs4686457 | TT | rs2862274 |
| 182 | TT | rs4686457 | CC | rs2862274 |
| 183 | TT | rs4686457 | TT | rs3749796 |
| 184 | AA | rs4686870 | AA | rs10919593 |
| 185 | AA | rs4686870 | CC | rs16902320 |
| 186 | AA | rs4686870 | CC | rs2862274 |
| 187 | AA | rs4686870 | TT | rs2862274 |
| 188 | AA | rs4686870 | AA | rs3749796 |
| 189 | AA | rs4686870 | TT | rs3749796 |
| 190 | AA | rs533391 | CG | rs2237342 |
| 191 | AT | rs533391 | CG | rs2237342 |
| 192 | CT | rs5979604 | AA | rs3749796 |
| 193 | GG | rs6066159 | CC | rs10278652 |
| 194 | CG | rs6066159 | CC | rs10278652 |
| 195 | GG | rs6066159 | AA | rs10919593 |
| 196 | CG | rs6066159 | AA | rs10919593 |
| 197 | GG | rs6066159 | AA | rs10920481 |
| 198 | CG | rs6066159 | AA | rs10920481 |
| 199 | TT | rs6094553 | CC | rs10278652 |
| 200 | AT | rs6094553 | CC | rs10278652 |
| 201 | TT | rs6851158 | GG | rs17120428 |
| 202 | AT | rs6851158 | GG | rs17120428 |
| 203 | AA | rs696405 | TT | rs2862274 |
| 204 | AA | rs7106605 | AA | rs3749796 |
| 205 | GG | rs7106605 | AA | rs3749796 |
| 206 | AA | rs7130910 | AA | rs3749796 |
| 207 | GG | rs7130910 | AA | rs3749796 |
| 208 | CC | rs7153013 | TT | rs17003277 |
| 209 | TT | rs7153013 | TT | rs17003277 |
| 210 | GG | rs7221263 | CC | rs11030746 |
| 211 | GG | rs7221263 | TT | rs11030746 |
| 212 | GG | rs7221263 | CC | rs11103184 |
| 213 | GG | rs7221263 | TT | rs11103184 |
| 214 | GG | rs7221263 | AA | rs16902320 |
| 215 | GG | rs7221263 | CC | rs17003277 |
| 216 | GG | rs7221263 | TT | rs17003277 |
| 217 | GG | rs7221263 | CC | rs17078925 |
| 218 | GG | rs7221263 | TT | rs17078925 |
| 219 | GG | rs7221263 | AA | rs17120428 |
| 220 | GG | rs7221263 | GG | rs17120428 |
| 221 | GG | rs7221263 | CC | rs2274703 |
| 222 | GG | rs7221263 | TT | rs2274703 |
| 223 | GG | rs7221263 | CC | rs2615903 |
| 224 | GG | rs7221263 | CC | rs2862274 |
| 225 | GG | rs7221263 | TT | rs2862274 |
| 226 | GG | rs7221263 | AA | rs3749796 |
| 227 | GG | rs7221263 | TT | rs3749796 |
| 228 | GG | rs7221263 | CC | rs3816612 |
| 229 | GG | rs7221263 | TT | rs3816612 |
| 230 | GG | rs7221263 | GG | rs4595891 |
| 231 | GG | rs7221263 | TT | rs4595891 |
| 232 | GG | rs7579939 | CC | rs2274703 |
| 233 | GG | rs7579939 | AA | rs3749796 |
| 234 | CC | rs999430 | CC | rs2274703 |
| 235 | CC | rs999430 | TT | rs2274703 |

wherein the presence of one or more of the combinations of a genotype at a single nucleotide polymorphism from column 1 and the corresponding genotype at a single nucleotide polymorphism from column 2 increases the subject's likelihood of developing an age-related disease.

A method of prophylactically treating a subject for an age-related disease comprising:

a) identifying the subject as likely to develop an age-related disease by, the method of any of claims 11-40; and then
b) administering a prophylactic therapy for the age-related disease to the subject, so as to thereby prophylactically treat the subject for the age-related disease.

### DETAILED DESCRIPTION OF THE INVENTION

A single nucleotide polymorphism (SNP) is a single base pair mutation at a specific locus, usually consisting of two alleles. Single nucleotide polymorphisms may be a substitution, deletion, or insertion to the sequence of the nucleic acid. Genotyping, which provides a measurement of the genetic variation between members of a species, has shown that single nucleotide polymorphisms (SNP) are the most common type of genetic variation. SNPs are often found to be the etiology of many human diseases and are becoming of particular interest in pharmacogenetics.

SNPs are referred to by their unique “rs” number which refers to the National Center for Biotechnology Information SNP cluster identifier. This database is searchable by rs number and includes information about the SNP and the surrounding genetic sequence.

As used herein, “detection” of a SNP shall mean identification of a single nucleotide polymorphism in a given position of a DNA obtained from a subject as determined by any of the well-known methods in the art, including, but not limited to, hybridization-based methods such as dynamic allele-specific hybridization, molecular beacons, and SNP microarrays, or enzyme-based methods such as restriction fragment length polymorphism, PCR-based methods, Flap endonuclease, primer extension, 5′-nuclease, or an oligonucleotide ligase assay.

The present invention provides a method for identifying a subject's likelihood of achieving longevity comprising detecting at least one of the following single nucleotide polymorphisms in a sample of a subject's DNA: nucleotide A at rs2410082, nucleotide A at rs11094258, nucleotide C at rs12131971, nucleotide C at rs966272, nucleotide G at rs6851158, nucleotide G at rs12556351, nucleotide G at rs5991963, nucleotide T at rs1047972, or nucleotide T at rs11001056,

wherein detection of the presence of one of more of the single nucleotide polymorphisms identifies the subject as likely to achieve of longevity.

rs11094256, rs966272 and rs12556351 are all located within the intron of gene HDAC8.

In an embodiment, the method comprises detecting at least one of the following single nucleotide polymorphisms: nucleotide A at rs11094258, nucleotide C at rs966272, or nucleotide G at rs12556351. In an embodiment, the method comprises detecting nucleotide A at rs2410082. In an embodiment, the method comprises detecting nucleotide C at rs12131971. In an embodiment, the method comprises detecting nucleotide G at rs6851158. In an embodiment, the method comprises detecting nucleotide G at rs5991963. In an embodiment, the method comprises detecting nucleotide T at rs1047972. In an embodiment, the method comprises detecting nucleotide T at rs11001056. In an embodiment, the method comprises detecting two or more of the single nucleotide polymorphisms.

In an embodiment, the method further comprises obtaining the sample of the subject's DNA from the subject prior to detecting the one or more single nucleotide polymorphisms.

As used herein, in humans, a subject “achieves longevity” by reaching at least eighty five years of age. Preferably, a subject who achieves longevity reaches at least ninety years of age. Most preferably, a subject who achieves longevity reaches at least ninety five years of age.

As used herein “likelihood of achieving longevity” is the propensity for, or probability that, a subject will live to 85 years of age or longer. A subject is “likely” to achieve longevity if the subject has a greater than 50% chance of living to 85 years of age or longer.

A subject having at least one of the above single nucleotide polymorphisms associated with longevity has a lower likelihood of developing an age-related disease than average, with a correspondingly higher likelihood of attaining exceptional longevity. The single nucleotide polymorphisms are located within the subject's DNA, either in the coding for a gene or in regulatory motifs. The presence of the particular nucleotide at the particular position within the gene or regulatory motifs may change gene expression or stability, affecting longevity. The present invention takes into account a subject's likelihood of longevity based on their genetic makeup. It should be bnorne in mind that life-style factors such as obesity will impact the likelihood of longevity. Appropriate correction can be made if desired.

The detection of two or more of the above-identified single nucleotide polymorphisms associated with longevity can further increase a subject's likelihood of achieving longevity.

The present invention also provides a method for identifying a subject's likelihood of developing an age-related disease comprising detecting at least one of the following single nucleotide polymorphisms in a sample of a subject's DNA: nucleotide A at rs16951040, nucleotide A at rs17625605, nucleotide A at rs3743180, nucleotide A at rs325380, nucleotide A at rs1528061, nucleotide C at rs17095079, nucleotide A at rs10775623, nucleotide A at rs10515725, nucleotide C at rs2061007, nucleotide A at rs4479454, nucleotide C at rs7630086, nucleotide C at rs3865868, nucleotide C at rs16987522, nucleotide C at rs16916036, nucleotide A at rs11818525, nucleotide A at rs17696856, nucleotide A at rs17014445, nucleotide T at rs1808723, nucleotide T at rs17049044, nucleotide G at rs11497898, nucleotide G at rs16916055, nucleotide T at rs2274703, nucleotide T at rs7067297, nucleotide G at rs2525841, nucleotide G at rs5951437, nucleotide T at rs1991921, nucleotide T at rs11247124, nucleotide T at rs3848883, or nucleotide G at rs5981402,

wherein detection of the presence of one or more of the single nucleotide polymorphisms in the subject's DNA identifies the subject as likely to develop an age-related disease

In an embodiment, the method comprises detecting at least one of the following single nucleotide polymorphisms: nucleotide A at rs3743180, or nucleotide T at rs1991921. In an embodiment, the method comprises detecting at least one of the following single nucleotide polymorphisms: nucleotide A at rs325380, nucleotide C at rs2061007, nucleotide T at rs1808723, or nucleotide T at rs11247124. In an embodiment, the method comprises detecting at least one of the following single nucleotide polymorphisms: nucleotide A at rs325380 or nucleotide C at rs2061007. In an embodiment, the method comprises detecting at least one of the following single nucleotide polymorphisms: nucleotide T at rs1808723 or nucleotide T at rs11247124. In an embodiment, the method comprises detecting at least one of the following single nucleotide polymorphisms: nucleotide C at rs16916036, or nucleotide G at rs16916055. In an embodiment, the method comprises detecting nucleotide A at rs16951040. In an embodiment, the method comprises detecting nucleotide A at rs17625605. In an embodiment, the method comprises detecting nucleotide A at rs1528061. In an embodiment, the method comprises detecting nucleotide C at rs17095079. In an embodiment, the method comprises detecting nucleotide A at rs10775623. In an embodiment, the method comprises detecting nucleotide A at rs10515725. In an embodiment, the method comprises detecting nucleotide A at rs4479454. In an embodiment, the method comprises detecting nucleotide C at rs7630086. In an embodiment, the method comprises detecting nucleotide C at rs3865868. In an embodiment, the method comprises detecting nucleotide C at rs16987522. In an embodiment, the method comprises detecting nucleotide A at rs11818525. In an embodiment, the method comprises detecting nucleotide A at rs17696856. In an embodiment, the method comprises detecting nucleotide A at rs17014445. In an embodiment, the method comprises detecting nucleotide T at rs17049044. In an embodiment, the method comprises detecting nucleotide G at rs11497898. In an embodiment, the method comprises detecting nucleotide T at rs2274703. In an embodiment, the method comprises detecting nucleotide T at rs7067297. In an embodiment, the method comprises detecting nucleotide G at rs2525841. In an embodiment, the method comprises detecting nucleotide G at rs5951437. In an embodiment, the method comprises detecting nucleotide T at rs3848883. In an embodiment, the method comprises detecting nucleotide G at rs5981402. In an embodiment, the method comprises detecting two or more of the single nucleotide polymorphisms.

The detection of two or more of the above-identified single nucleotide polymorphisms associated with deleterious effects can increase a subject's likelihood of developing an age-related disease.

rs3743180 and rs1991921 are both located in the intron of gene MEF2A. rs328380, rs2061007, rs1808723 and rs11247124 are all located within gene LYSMD4. rs325380 and rs2061007 are located within the coding sequence of gene LYSMD4 while rs1808723 and rs11247124 are located within the intron of gene LYSMD4. rs16916036 and rs16916055 are located upstream of gene FAT3.

An “age-related disease” as used herein is any disease whose prevalence increases with an increase in a subject's age. Age-related diseases include cardiovascular diseases such as hypertension, myocardial infarctions, strokes, and transient ischemic attacks; as well as diabetes mellitus, Alzheimer's disease, and cancers.

As used herein “likelihood of developing an age-related disease” is the propensity for, or probability that, a subject will develop an age-related disease. A subject is “likely” to develop an age-related disease if the subject has a greater than 50% chance of experiencing the age-related disease.

A subject having at least one of the above-identified single nucleotide polymorphisms has a higher likelihood of developing an age-related disease, with a correspondingly lower likelihood of attaining longevity. The single nucleotide polymorphisms are located within the subject's DNA, including in the coding portion(s) of a gene or in a regulatory motif. The presence of the particular nucleotide at the particular position within the gene or regulatory motifs may change gene expression or stability, affecting longevity.

The deleterious impact on a subject's longevity of the presence of one or more deleterious SNPs may be mitigated by the presence of a buffering SNP. Such buffering SNPs affect genes which buffer the effect of one or more deleterious genes. The buffer-deleterious relationship is specific and limited with only certain SNP genotypes, and by extension, their related genes, buffering only certain deleterious SNP genotypes. The presence in a single subject of both a buffered SNP genotype and the buffering SNP genotype to which it corresponds results in a decreased likelihood of that subject developing an age-related disease than if only the deleterious SNP genotype was present.

In an embodiment, the age-related disease is a cardiovascular disease, diabetes mellitus, Alzheimer's disease, or a cancer. In an embodiment, the age-related disease is cardiovascular disease and is hypertension, myocardial infarction, stroke, or transient ischemic attack.

The present invention further provides a method for identifying a subject's likelihood of achieving longevity comprising detecting in a sample of the subject's DNA at least one of the following combinations of a genotype at a single nucleotide polymorphism from column 1 and a corresponding genotype at a single nucleotide polymorphism from column 2:

| | |
|---|---|
| Column 1 | Column 2 |

| | Genotype | dbSNP | Genotype | dbSNP |
|---|---|---|---|---|
| 1 | CG | rs10041500 | AG | rs5912192 |
| 2 | CC | rs10738555 | GG | rs10919593 |
| 3 | GC | rs10738555 | GG | rs10919593 |
| 4 | TT | rs10919593 | CT | rs642053 |
| 5 | AT | rs10919593 | CT | rs642053 |
| 6 | CT | rs11738547 | CC | rs11030746 |
| 7 | CT | rs11738547 | TT | rs11030746 |
| 8 | CT | rs11738547 | AA | rs1257689 |
| 9 | CT | rs11738547 | GG | rs17120428 |
| 10 | CT | rs11738547 | AA | rs3749796 |
| 11 | CT | rs11738547 | TT | rs3749796 |
| 12 | TT | rs12276840 | AC | rs10227794 |
| 13 | TT | rs12276840 | CC | rs10227794 |
| 14 | TT | rs12276840 | CC | rs2274703 |
| 15 | CC | rs12276840 | CC | rs2274703 |
| 1 | CC | rs12348066 | GG | rs496666 |
| 17 | CG | rs12348066 | GG | rs496666 |
| 18 | CC | rs12348066 | CC | rs536789 |
| 19 | CG | rs12348066 | CC | rs536789 |
| 20 | CC | rs12348066 | GG | rs5912192 |
| 21 | CG | rs12348066 | GG | rs5912192 |
| 22 | CC | rs12348066 | TT | rs7063827 |
| 23 | CG | rs12348066 | TT | rs7063827 |
| 24 | CC | rs12432648 | CT | rs12838423 |
| 25 | GG | rs12432648 | CT | rs12838423 |
| 26 | CC | rs12432648 | GT | rs12857956 |
| 27 | GG | rs12432648 | GT | rs12857956 |
| 28 | AG | rs1255575 | CC | rs2862274 |
| 29 | AG | rs1255575 | TT | rs2862274 |
| 30 | CC | rs1255575 | CC | rs2862274 |
| 31 | CG | rs1255575 | CC | rs2862274 |
| 32 | CC | rs1255575 | TT | rs2862274 |
| 33 | CG | rs1255575 | TT | rs2862274 |
| 34 | GG | rs1430707 | TT | rs2274703 |
| 35 | GG | rs1430707 | CC | rs2862274 |
| 36 | GG | rs1430707 | TT | rs2862274 |
| 37 | GG | rs1430707 | AA | rs3749796 |
| 38 | GG | rs1461809 | TT | rs3816612 |
| 39 | CC | rs1499953 | AA | rs10919593 |
| 40 | AG | rs1528708 | GG | rs4595891 |
| 41 | TT | rs16880188 | CC | rs11030746 |
| 42 | AT | rs16880188 | CC | rs11030746 |
| 43 | TT | rs16880188 | TT | rs11030746 |
| 44 | AT | rs16880188 | TT | rs11030746 |
| 45 | TT | rs16880188 | CC | rs17003277 |
| 46 | AT | rs16880188 | CC | rs17003277 |
| 47 | TT | rs16880188 | TT | rs17003277 |
| 48 | AT | rs16880188 | TT | rs17003277 |
| 49 | TT | rs16951040 | CC | rs1324362 |
| 50 | AT | rs16951040 | CC | rs1324362 |
| 51 | CC | rs16951040 | AG | rs17154320 |
| 52 | TT | rs17014445 | GG | rs10919593 |
| 53 | AT | rs17014445 | GG | rs10919593 |
| 54 | TT | rs17014445 | GG | rs10919593 |
| 55 | GG | rs17014445 | AA | rs10919593 |
| 56 | GG | rs17014445 | GG | rs10919593 |
| 57 | GG | rs17014445 | AA | rs10919593 |
| 58 | TT | rs17014445 | CC | rs11103184 |
| 59 | AT | rs17014445 | CC | rs11103184 |
| 60 | GG | rs17014445 | CC | rs11103184 |
| 61 | TT | rs17014445 | CC | rs11103512 |
| 62 | AT | rs17014445 | CC | rs11103512 |
| 63 | GG | rs17014445 | CC | rs11103512 |
| 64 | TT | rs17014445 | CC | rs11183915 |
| 65 | AT | rs17014445 | CC | rs11183915 |
| 66 | GG | rs17014445 | CC | rs11183915 |
| 67 | GG | rs17014445 | AG | rs17154320 |
| 68 | TT | rs17014445 | TT | rs1822910 |
| 69 | AT | rs17014445 | TT | rs1822910 |
| 70 | GG | rs17014445 | TT | rs1822910 |
| 71 | GG | rs17014445 | AA | rs2525841 |
| 72 | GG | rs17014445 | AA | rs3749796 |
| 73 | GG | rs17014445 | AA | rs3925518 |
| 74 | GG | rs17014445 | TT | rs6077004 |
| 75 | GG | rs17014445 | CT | rs6419449 |
| 76 | GG | rs17014445 | CG | rs6540375 |
| 77 | GG | rs17014445 | AT | rs6641231 |
| 78 | GG | rs17014445 | CC | rs7827228 |
| 79 | TT | rs17014445 | CT | rs9586459 |
| 80 | AT | rs17014445 | CT | rs9586459 |
| 81 | TT | rs17014445 | TT | rs9586459 |
| 82 | AT | rs17014445 | TT | rs9586459 |
| 83 | GG | rs17014445 | CT | rs9586459 |
| 84 | GG | rs17014445 | TT | rs9586459 |
| 85 | AA | rs17049044 | CT | rs3859965 |
| 86 | AT | rs17049044 | CT | rs3859965 |
| 87 | CC | rs17049044 | AA | rs553369 |
| 88 | AA | rs17083201 | GT | rs2144497 |
| 89 | AT | rs17083201 | GT | rs2144497 |
| 90 | AA | rs17154320 | CC | rs10278652 |
| 91 | GG | rs17154320 | CC | rs10278652 |
| 92 | AA | rs17372695 | CC | rs2862274 |
| 93 | AT | rs17372695 | CC | rs2862274 |
| 94 | AA | rs17372695 | TT | rs2862274 |
| 95 | AT | rs17372695 | TT | rs2862274 |
| 96 | CT | rs17775764 | AG | rs5912192 |
| 97 | TT | rs1822910 | AC | rs11625236 |
| 98 | TT | rs1822910 | CC | rs2274703 |
| 99 | TT | rs1822910 | CT | rs2613216 |
| 100 | TT | rs1822910 | TT | rs2613216 |
| 101 | TT | rs1822910 | AA | rs2623149 |
| 102 | TT | rs1822910 | AG | rs2623149 |
| 103 | TT | rs1822910 | GG | rs5981167 |
| 104 | AT | rs1822910 | GG | rs5981167 |
| 105 | AA | rs1991921 | CC | rs11030746 |
| 106 | AT | rs1991921 | CC | rs11030746 |
| 107 | AA | rs1991921 | TT | rs11030746 |
| 108 | AT | rs1991921 | TT | rs11030746 |
| 109 | AA | rs1991921 | TT | rs3749796 |
| 110 | AT | rs1991921 | TT | rs3749796 |
| 111 | AA | rs1991921 | CC | rs3816612 |
| 112 | AT | rs1991921 | CC | rs3816612 |
| 113 | GG | rs2012129 | CC | rs11030746 |
| 114 | CG | rs2012129 | CC | rs11030746 |
| 115 | GG | rs2012129 | TT | rs11030746 |
| 116 | CG | rs2012129 | TT | rs11030746 |
| 117 | GG | rs2012129 | TT | rs3749796 |
| 118 | CG | rs2012129 | TT | rs3749796 |
| 119 | GG | rs2061007 | CC | rs11030746 |
| 120 | CG | rs2061007 | CC | rs11030746 |
| 121 | GG | rs2061007 | TT | rs11030746 |
| 122 | CG | rs2061007 | TT | rs11030746 |
| 123 | GG | rs2061007 | AA | rs16902320 |
| 124 | CG | rs2061007 | AA | rs16902320 |
| 125 | GG | rs2061007 | CC | rs16902320 |
| 126 | CG | rs2061007 | CC | rs16902320 |
| 127 | GG | rs2061007 | CC | rs17003277 |
| 128 | CG | rs2061007 | CC | rs17003277 |
| 129 | GG | rs2061007 | TT | rs1822910 |
| 130 | CG | rs2061007 | TT | rs1822910 |
| 131 | GG | rs2061007 | TT | rs2274703 |
| 132 | CG | rs2061007 | TT | rs2274703 |
| 133 | GG | rs2061007 | CC | rs2862274 |
| 134 | CG | rs2061007 | CC | rs2862274 |
| 135 | GG | rs2061007 | TT | rs2862274 |
| 136 | CG | rs2061007 | TT | rs2862274 |
| 137 | GG | rs2061007 | TT | rs3749796 |
| 138 | CG | rs2061007 | TT | rs3749796 |
| 139 | GG | rs2061007 | CC | rs3816612 |
| 140 | CG | rs2061007 | CC | rs3816612 |
| 141 | GG | rs2061007 | TT | rs3816612 |
| 142 | CG | rs2061007 | TT | rs3816612 |
| 143 | GG | rs2098846 | CC | rs17003277 |
| 144 | CG | rs2098846 | CC | rs17003277 |
| 145 | GG | rs2098846 | TT | rs17003277 |
| 146 | CG | rs2098846 | TT | rs17003277 |
| 147 | GG | rs2098846 | TT | rs2274703 |
| 148 | CG | rs2098846 | TT | rs2274703 |
| 149 | CC | rs2274703 | CG | rs2237342 |
| 150 | AA | rs2274703 | CG | rs2237342 |
| 151 | AT | rs2274703 | CG | rs2237342 |
| 152 | CC | rs2274703 | CC | rs7827228 |
| 153 | AG | rs2382993 | CC | rs11103184 |
| 154 | AA | rs2525841 | CT | rs12838423 |
| 155 | AA | rs2525841 | GT | rs12857956 |
| 156 | CC | rs2525841 | AG | rs5981167 |
| 157 | CG | rs2525841 | AG | rs5981167 |
| 158 | CC | rs2525841 | GG | rs5981167 |
| 159 | CG | rs2525841 | GG | rs5981167 |
| 160 | TT | rs2615903 | AG | rs17154320 |
| 161 | TT | rs2615903 | AA | rs2525841 |
| 162 | TT | rs2615903 | AA | rs3749796 |
| 163 | AA | rs2762933 | CC | rs2862274 |
| 164 | AT | rs2762933 | CC | rs2862274 |
| 165 | AA | rs2762933 | TT | rs2862274 |
| 166 | AT | rs2762933 | TT | rs2862274 |
| 167 | TT | rs325380 | CC | rs11030746 |
| 168 | AT | rs325380 | CC | rs11030746 |
| 169 | TT | rs325380 | TT | rs11030746 |
| 170 | AT | rs325380 | TT | rs11030746 |
| 171 | TT | rs325380 | TT | rs3749796 |
| 172 | AT | rs325380 | TT | rs3749796 |
| 173 | CC | rs368866 | CC | rs17003277 |
| 174 | CG | rs368866 | CC | rs17003277 |
| 175 | CC | rs368866 | TT | rs17003277 |
| 176 | CG | rs368866 | TT | rs17003277 |
| 177 | TT | rs4686457 | AA | rs10919593 |
| 178 | TT | rs4686457 | TT | rs11030746 |
| 179 | TT | rs4686457 | AA | rs16902320 |
| 180 | TT | rs4686457 | CC | rs16902320 |
| 181 | TT | rs4686457 | TT | rs2862274 |
| 182 | TT | rs4686457 | CC | rs2862274 |
| 183 | TT | rs4686457 | TT | rs3749796 |
| 184 | AA | rs4686870 | AA | rs10919593 |
| 185 | AA | rs4686870 | CC | rs16902320 |
| 186 | AA | rs4686870 | CC | rs2862274 |
| 187 | AA | rs4686870 | TT | rs2862274 |
| 188 | AA | rs4686870 | AA | rs3749796 |
| 189 | AA | rs4686870 | TT | rs3749796 |
| 190 | AA | rs533391 | CG | rs2237342 |
| 191 | AT | rs533391 | CG | rs2237342 |
| 192 | CT | rs5979604 | AA | rs3749796 |
| 193 | GG | rs6066159 | CC | rs10278652 |
| 194 | CG | rs6066159 | CC | rs10278652 |
| 195 | GG | rs6066159 | AA | rs10919593 |
| 196 | CG | rs6066159 | AA | rs10919593 |
| 197 | GG | rs6066159 | AA | rs10920481 |
| 198 | CG | rs6066159 | AA | rs10920481 |
| 199 | TT | rs6094553 | CC | rs10278652 |
| 200 | AT | rs6094553 | CC | rs10278652 |
| 201 | TT | rs6851158 | GG | rs17120428 |
| 202 | AT | rs6851158 | GG | rs17120428 |
| 203 | AA | rs696405 | TT | rs2862274 |
| 204 | AA | rs7106605 | AA | rs3749796 |
| 205 | GG | rs7106605 | AA | rs3749796 |
| 206 | AA | rs7130910 | AA | rs3749796 |
| 207 | GG | rs7130910 | AA | rs3749796 |
| 208 | CC | rs7153013 | TT | rs17003277 |
| 209 | TT | rs7153013 | TT | rs17003277 |
| 210 | GG | rs7221263 | CC | rs11030746 |
| 211 | GG | rs7221263 | TT | rs11030746 |
| 212 | GG | rs7221263 | CC | rs11103184 |
| 213 | GG | rs7221263 | TT | rs11103184 |
| 214 | GG | rs7221263 | AA | rs16902320 |
| 215 | GG | rs7221263 | CC | rs17003277 |
| 216 | GG | rs7221263 | TT | rs17003277 |
| 217 | GG | rs7221263 | CC | rs17078925 |
| 218 | GG | rs7221263 | TT | rs17078925 |
| 219 | GG | rs7221263 | AA | rs17120428 |
| 220 | GG | rs7221263 | GG | rs17120428 |
| 221 | GG | rs7221263 | CC | rs2274703 |
| 222 | GG | rs7221263 | TT | rs2274703 |
| 223 | GG | rs7221263 | CC | rs2615903 |
| 224 | GG | rs7221263 | CC | rs2862274 |
| 225 | GG | rs7221263 | TT | rs2862274 |
| 226 | GG | rs7221263 | AA | rs3749796 |
| 227 | GG | rs7221263 | TT | rs3749796 |
| 228 | GG | rs7221263 | CC | rs3816612 |
| 229 | GG | rs7221263 | TT | rs3816612 |
| 230 | GG | rs7221263 | GG | rs4595891 |
| 231 | GG | rs7221263 | TT | rs4595891 |
| 232 | GG | rs7579939 | CC | rs2274703 |
| 233 | GG | rs7579939 | AA | rs3749796 |
| 234 | CC | rs999430 | CC | rs2274703 |
| 235 | CC | rs999430 | TT | rs2274703 |

wherein the presence of one or more of the combinations of the recited genotype at the single nucleotide polymorphism from column 1 and the corresponding recited genotype at the single nucleotide polymorphism from column 2 indicates that the subject is likely to develop an age-related disease.

The present invention further provides a method for identifying a subject's likelihood of achieving longevity comprising detecting at least one of the following genotypes in a sample of a subject's DNA at a single nucleotide polymorphism: genotype CC at rs6470902, genotype GC at rs6470902, genotype BB at rs7168796, genotype BB at rs16853867, genotype AA at rs17049044, genotype AA at rs11020277, genotype AA at rs11020294, genotype AA at rs7653267, genotype AA at rs11909021, genotype AB at rs12010050, genotype AB at rs9488847, genotype AB at rs1255575, genotype AB at rs16895959, genotype AB at rs716682, genotype BB at rs2289507, genotype BB at rs17043069, or genotype BB at rs7038833,

wherein the presence of one or more of the genotypes at a single nucleotide polymorphism increases the subject's likelihood of longevity.

The present invention further provides a method for identifying a subject's likelihood of developing an age-related disease comprising detecting at least one of the following genotypes at a single nucleotide polymorphism in a sample of the subject's DNA: genotype CC at rs9914076, genotype CC at rs16971905, genotype CC at rs7272951, genotype CC at rs6093591, genotype CG at rs509193, genotype AA at rs11164749, genotype AA at rs1712676, genotype AA at rs12521591, genotype AA at rs7168796, genotype AA at rs16853867, genotype AB at rs16929656, genotype AB at rs3814284, genotype AB at rs6009526, genotype AB at rs7780235, genotype AB at rs2206100, genotype BB at rs16895959, genotype BB at rs716682, genotype BB at rs702743, genotype BB at rs13355963, or genotype BB at rs16860657, wherein the presence of one or more of the genotypes at a single nucleotide polymorphism increases the subject's likelihood of developing an age-related disease.

In an embodiment of the methods described herein, the method further comprises obtaining the sample of the subject's DNA from the subject prior to detecting the one or more single nucleotide polymorphisms.

The present invention further provides a method of prophylactically treating a subject for an age-related disease comprising:

a) identifying the subject as likely to develop an age-related disease by the method of any of claims 11-40; and then
b) administering a prophylactic therapy for the age-related disease to the subject, so as to thereby prophylactically treat the subject for the age-related disease.

In an embodiment, the age-related disease is a cardiovascular disease, diabetes mellitus, Alzheimer's disease, or a cancer. In an embodiment, the age-related disease is cardiovascular disease and is hypertension, myocardial infarction, stroke, or transient ischemic attack.

In the methods described herein, the subject in may be any animal. Preferably the subject is a mammal. More preferably the subject is a human.

### Experimental Details

### 1. Methods and Materials

Study design/subjects: Three hundred sixty five Ashkenazi Jewish centenarians (probands, 265 women, median age, 98 years and 90 men, median age 98 years), and 122 of their offsprings' spouses ([control group A] 56 women, median age, 67 years and 66 men, median age, 74 years), were recruited randomly to participate in the study. An additional control group of 129 Ashkenazi Jews ([control group B] 92 women, median age, 78 years and 37 men, median age, 76 years) not family-related to the above subjects but from the same geographic area was similarly assessed, for a total of 615 individuals. Stated age was verified by checking birth certificates or U.S. passports in all subjects. Information on medical history, demographic characteristics and clinical data were obtained uniformly using a structured questionnaire. All subjects underwent a physical examination and were asked to provide a blood sample. Informed written consent was obtained in accordance with the policy of the Committee on Clinical Investigation of the Albert Einstein College of Medicine. Participants in the control groups were excluded if their biological parents achieved longevity (i.e. lived to >85 years). DNA specimens were processed for the genome scanning in both younger and older cohort. In addition, the 6.0 Affymetrix® platform was employed (1.8 million single nucleotide polymorphisms and copy number variants, see below Affymetrix® hybridization protocol) in specimens of 251 younger unrelated controls (F/M 148/103) and 364 centenarians (F/M 264/99) (Table 1). High call rate (99.6%) and expected rate of heterozygosity (26.5%) across the analyzed 615 arrays assure the quality, accuracy, and reproducibility of the results (Table 2).

Procedures: The statistical approach addresses two major challenges: (a)

TABLE 1
population demography.

| Group | Gender | No. of chip | Age range |
|---|---|---|---|
| Control | Female | 148 | 43-94 |
| Control | Male | 103 | 56-86 |
| Total | | 251 | |
| Proband | Female | 265 | 95-109 |
| Proband | Male | 99 | 95-106 |
| Total | | 364 | |

appropriately controlling Type I and type II errors following multiple comparisons adjustment; and (b) dissecting the genetic components of complex quantitative traits (Longevity), specifically teasing out whether there are susceptibility disease loci that are more prevalent among elderly cohort versus loci related to maintaining health that are more prevalent among centenarian.

TABLE 2
Assay characteristic and quality

| Quality Control | Range (%) | Observed (%) |
|---|---|---|
| Hybridization success | 95 | 96 |
| Call rate | >98 | 99.6 |
| Heterozygote rate | 30 | 26.1 |
| Homozygote rate | 70 | 73.5 |
| QC Call Rate | >98 | 99.1 |
| Contrast QC (Random) | >=0.4 | 2.7 |
| Y Chr. in female | 32 | 29.3 |

A systematic approach was used incorporating four major steps. Step 1-Genome wide association between each of more than 906 k SNPs and longevity in case-control manner using the Ashkenazi cohort to detect significant higher prevalence either in control (deleterious gene) or centenarian (longevity gene). Since power of identifying important SNPs depends on the analysis approach (single SNP analysis and multi-locus analysis) and the relative efficiency of them depends on the unknown underlying genetic mechanism of velocity, screening was not limited to any single analysis approach. Step 2-Trend analysis on the most significant SNPs resulted from the initial screening, where SNPs' genotypes defined as 0, 1 and 2 corresponded to Homozygote for the first allele heterozygote and homozygote for the second allele versus age. Step 3—Genome wide screening for buffering pattern (U-shape distribution) by using a binomial model with an identity link function and both linear and quadratic terms for age, and test for the significant quadratic component. The candidate variants were then subjected to gene-gene interaction with longevity-associated genes detected in the first screening. Step 4—Association of the initial candidate variants with favorable healthy phenotypes. The last step required the inclusion of offspring of centenarian.

### 2. Results

A substantial amount of genotype was generated using the 6.0 Affymetrix® platform. 364 centenarians (73% female) and 251 unrelated controls (59% female) were genotyped for 906600 SNPs each. Out of 906600 available SNPs, 58860 were not polymorphic (6.5%) in the studied population and only 46677 (5.1%) of the remainder passed significant threshold (p<0.05). Applying FDR (p=0.1) multi-comparison correction SNPs were identified whose minor allele frequencies was significantly higher or lower among control compared to probands. 9 SNPs whose minor allele frequency was significantly higher among probands compared to control (“longevity-genes”), and 29 SNPs whose minor allele frequency was significantly higher among controls compared to probands (“deleterious-genes”). Trend analysis was then applied where SNPs' alleles defined as 0, 1 and 2 corresponded to Homozygote for the first allele heterozygote and homozygote for the second allele versus age. Only a few SNPs passed the significant threshold which increased the validity of the framework pattern (Table 3). Additionally, some longevity and deleterious genotypes were determined. (Table 4, 5). The presence of the determined genotype at the SNP has a stastically significant association with a subject's likelihood of longevity or of developing an age-related disease.

TABLE 3
Characteristic of the deleterious and longevity genes.

| | | | | | | | | Minor Allele | | | | |
|---|---|---|---|---|---|---|---|---|---|---|---|---|
| | Affy. | | | | | | | Freq. | | P-value (C-C) | | |
| Type | Marker | dbSNP | Gene | Chr. | Physical Position | Loc | Loc-Pos | Proband | Control | Allele | Gene | Trend |
| Deleterious | 8598768 | rs3848883 | NLGN4X | X | 5171554 | up | 646529 | 0.03 | 0.09 | 4.7E−6 | 9.6E−4 | 5.2E−4 |
| Deleterious | 8331392 | rs7067297 | JARID1D | Y | 20449645 | down | 588111 | 0.19 | 0.42 | 3.7E−6 | 1.1E−3 | 1.1E−3 |
| Deleterious | 8393388 | rs5951437 | ZNF645 | X | 22280656 | down | 78161 | 0.05 | 0.13 | 6.9E−6 | 5.1E−4 | 1.8E−4 |
| Deleterious | 8610848 | rs5981402 | MAGEE2 | X | 75195977 | up | 274181 | 0.00 | 0.04 | 4.9E−6 | 1.2E−3 | 1.2E−3 |
| Deleterious | 8393550 | rs16987522 | AMOT | X | 112905334 | up | 934635 | 0.02 | 0.07 | 3.5E−6 | 1.0E−3 | 1.0E−3 |
| Deleterious | 8316256 | rs3865868 | | Y | 2147483647 | | | 0.22 | 0.38 | 7.4E−10 | 5.0E−5 | 1.0E−5 |
| Deleterious | 2077686 | rs17625605 | FAM5B | 01 | 175895442 | down | 377869 | 0.14 | 0.26 | 1.0E−7 | 3.3E−4 | 1.2E−4 |
| Deleterious | 8300818 | rs2274703 | LEMD1 | 01 | 203620091 | CDS | 0 | 0.10 | 0.19 | 5.0E−6 | 4.8E−5 | 7.6E−4 |
| Deleterious | 4298161 | rs4479454 | SNTG2 | 02 | 1091472 | intron | 0 | 0.14 | 0.26 | 2.6E−6 | 3.2E−4 | 8.3E−4 |
| Deleterious | 8687186 | rs17014445 | MYADML | 02 | 34058603 | up | 252257 | 0.06 | 0.21 | 1.6E−13 | 7.8E−7 | 1.1E−7 |
| Deleterious | 4207169 | rs1528061 | TMEM182 | 02 | 103102064 | up | 1413599 | 0.04 | 0.13 | 1.1E−6 | 6.3E−4 | 1.4E−4 |
| Deleterious | 8310986 | rs7630086 | BCL6 | 03 | 189369432 | up | 423263 | 0.03 | 0.09 | 7.3E−6 | 5.3E−5 | 9.8E−6 |
| Deleterious | 1850528 | rs17049044 | PCDH18 | 04 | 137958761 | down | 700761 | 0.05 | 0.17 | 1.9E−9 | 4.5E−5 | 1.7E−5 |
| Deleterious | 4265328 | rs10515725 | MRPL22 | 05 | 154457096 | up | 130372 | 0.40 | 0.44 | 1.7E−7 | 3.4E−4 | 1.7E−4 |
| Deleterious | 1950750 | rs11497898 | REEP3 | 10 | 66188858 | down | 1856145 | 0.01 | 0.09 | 1.0E−9 | 4.0E−5 | 8.3E−6 |
| Deleterious | 8639653 | rs11818525 | STK32C | 10 | 133965422 | intron | 0 | 0.05 | 0.20 | 4.0E−12 | 4.4E−6 | 7.0E−7 |
| Deleterious | 8513633 | rs16916036 | FAT3 | 11 | 90768021 | up | 956906 | 0.00 | 0.03 | 5.4E−7 | 4.5E−7 | 4.5E−7 |
| Deleterious | 2013530 | rs16916055 | FAT3 | 11 | 90799677 | up | 925247 | 0.00 | 0.04 | 2.6E−7 | 2.1E−7 | 2.1E−7 |
| Deleterious | 4219182 | rs17095079 | CNTN5 | 11 | 99849414 | down | 116731 | 0.28 | 0.41 | 4.6E−6 | 4.4E−3 | 1.1E−3 |
| Deleterious | 8686344 | rs17696856 | PRMT8 | 12 | 3484816 | intron | 0 | 0.36 | 0.49 | 7.2E−6 | 1.7E−5 | 1.9E−5 |
| Deleterious | 8347387 | rs2525841 | ACSS3 | 12 | 80054239 | intron | 0 | 0.11 | 0.22 | 2.2E−6 | 6.3E−4 | 2.2E−4 |
| Deleterious | 8453929 | rs1991921 | MEF2A | 15 | 98011512 | intron | 0 | 0.39 | 0.47 | 3.9E−6 | 3.8E−6 | 3.2E−6 |
| Deleterious | 2083837 | rs3743180 | MEF2A | 15 | 98016953 | intron | 0 | 0.34 | 0.47 | 9.4E−6 | 9.6E−6 | 3.6E−6 |
| Deleterious | 2310343 | rs325380 | LYSMD4 | 15 | 98074141 | CDS | 0 | 0.32 | 0.45 | 5.3E−6 | 1.1E−5 | 5.5E−6 |
| Deleterious | 1830197 | rs1808723 | LYSMD4 | 15 | 98080351 | intron | 0 | 0.27 | 0.39 | 4.0E−6 | 1.8E−5 | 3.3E−6 |
| Deleterious | 8598001 | rs11247124 | LYSMD4 | 15 | 98083248 | intron | 0 | 0.19 | 0.31 | 3.0E−6 | 1.2E−5 | 1.9E−6 |
| Deleterious | 4289735 | rs2061007 | LYSMD4 | 15 | 98087203 | CDS | 0 | 0.31 | 0.44 | 1.3E−6 | 1.8E−6 | 1.4E−6 |
| Deleterious | 2051880 | rs16951040 | TOX3 | 16 | 50892007 | down | 138634 | 0.14 | 0.31 | 8.1E−11 | 7.7E−6 | 3.6E−6 |
| Deleterious | 4247554 | rs10775623 | FAM129C | 19 | 17501317 | intron | 0 | 0.01 | 0.05 | 1.3E−6 | 4.4E−4 | 9.4E−5 |
| Longevity | 8381426 | rs11094258 | HDAC8 | X | 71503499 | intron | 0 | 0.14 | 0.05 | 2.9E−7 | 1.1E−4 | 2.2E−5 |
| Longevity | 1902577 | rs966272 | HDAC8 | X | 71503967 | intron | 0 | 0.15 | 0.06 | 1.9E−6 | 1.5E−4 | 5.3E−5 |
| Longevity | 8305225 | rs12556351 | HDAC8 | X | 71509736 | intron | 0 | 0.15 | 0.06 | 1.3E−6 | 1.3E−4 | 3.7E−5 |
| Longevity | 8565471 | rs5991963 | TAF7L | X | 100450759 | up | 16058 | 0.43 | 0.30 | 8.5E−6 | 4.8E−4 | 9.5E−5 |
| Longevity | 8653083 | rs12131971 | POU3F1 | 01 | 38582167 | up | 52751 | 0.50 | 0.36 | 3.4E−6 | 2.7E−5 | 5.5E−6 |
| Longevity | 4297368 | rs6851158 | RPS26 | 04 | 113998568 | down | 356083 | 0.34 | 0.15 | 3.7E−10 | 1.2E−5 | 4.2E−6 |
| Longevity | 4204502 | rs2410082 | SGCZ | 08 | 13526989 | up | 694347 | 0.30 | 0.14 | 1.1E−9 | 4.9E−5 | 9.7E−6 |
| Longevity | 8651271 | rs11001056 | PRKG1 | 10 | 53599797 | intron | 0 | 0.46 | 0.41 | 5.5E−6 | 4.3E−5 | 7.9E−6 |
| Longevity | 8579423 | rs1047972 | AURKA | 20 | 54394870 | CDS | 0 | 0.49 | 0.34 | 4.9E−7 | 8.3E−4 | 1.8E−4 |

TABLE 4

| | | Afy. | | | | Physical | | Loc- |
|---|---|---|---|---|---|---|---|---|
| Genetype | Type | Marker | dbSNP | Gene | Chr. | Position | Loc | Pos |
| CC | Longevity | 8425953 | rs6470902 | ADCY8 | 08 | 132297207 | down | 216998 |
| GC | Longevity | 8425953 | rs6470902 | ADCY8 | 08 | 132297207 | down | 216998 |
| CC | Deleterious | 4297701 | rs9914076 | MFSD11 | 17 | 72251995 | intron | 0 |
| CC | Deleterious | 8327158 | rs16971905 | C16orf47 | 16 | 71982564 | up | 246717 |
| CC | Deleterious | 8552632 | rs7272951 | PAX1 | 20 | 21618645 | up | 15719 |
| CC | Deleterious | 8683414 | rs6093591 | PTPRT | 20 | 40264813 | intron | 0 |
| CG | Deleterious | 8379307 | rs509193 | FGF14 | 13 | 101618897 | intron | 0 |

TABLE 5

| | dbSNP RS | SNP | Physical | Gene | Gene | | |
|---|---|---|---|---|---|---|---|
| Probe Set ID | ID | Chr. | Position | Pos. | Distance | Gene | Trend |
| SNP_A-1800912 | rs11164749 | 1 | 103476511 | up | 422333 | AMY2B | AA− |
| SNP_A-1800912 | rs11164749 | 1 | 103476511 | up | 129871 | COL11A1 | AA− |
| SNP_A-2292607 | rs17121676 | 12 | 57571727 | intron | 0 | LRIG3 | AA− |
| SNP_A-8684973 | rs12521591 | 5 | 165433445 | up | 1210975 | ODZ2 | AA− |
| SNP_A-8684973 | rs12521591 | 5 | 165433445 | up | 44531 | RPL7 | AA− |
| SNP_A-8684973 | rs12521591 | 5 | 165433445 | up | 2218224 | WWC1 | AA− |
| SNP_A-1960483 | rs7168796 | 15 | 76587549 | intron | 0 | AGPHD1 | AA−/ |
| | | | | | | | BB+ |
| SNP_A-2253746 | rs16853867 | 1 | 14478589 | up | 319210 | RP1- | AA−/ |
| | | | | | | 21O18.1 | BB+ |
| SNP_A-2253746 | rs16853867 | 1 | 14478589 | down | 454430 | PRDM2 | AA−/ |
| | | | | | | | BB+ |
| SNP_A-1850528 | rs17049044 | 4 | 137958761 | down | 700764 | PCDH18 | AA+ |
| SNP_A-1850528 | rs17049044 | 4 | 137958761 | up | 461819 | TERF1 | AA+ |
| SNP_A-1913421 | rs11020277 | 11 | 92722365 | intron | 0 | CCDC67 | AA+ |
| SNP_A-2226653 | rs11020294 | 11 | 92754811 | intron | 0 | CCDC67 | AA+ |
| SNP_A-2238221 | rs7653267 | 3 | 13556247 | up | 9377 | FBLN2 | AA+ |
| SNP_A-2238221 | rs7653267 | 3 | 13556247 | down | 33323 | HDAC11 | AA+ |
| SNP_A-8318414 | rs11919021 | 3 | 194341883 | up | 99728 | HRASLS | AA+ |
| SNP_A-8318414 | rs11919021 | 3 | 194341883 | up | 223239 | C3orf59 | AA+ |
| SNP_A-1907137 | rs16929656 | 11 | 3886562 | intron | 0 | STIM1 | AB− |
| SNP_A-2064770 | rs3814284 | 16 | 48806581 | intron | 0 | PAPD5 | AB− |
| SNP_A-8400133 | rs6009526 | 22 | 47955942 | down | 443351 | C22orf34 | AB− |
| SNP_A-8400133 | rs6009526 | 22 | 47955942 | down | 422194 | FAM19A5 | AB− |
| SNP_A-8400133 | rs6009526 | 22 | 47955942 | down | 596992 | BRD1 | AB− |
| SNP_A-8564841 | rs7780235 | 7 | 16712241 | 3UTR | 0 | BZW2 | AB− |
| SNP_A-8626264 | rs2206100 | 6 | 19300977 | up | 644618 | ID4 | AB− |
| SNP_A-8626264 | rs2206100 | 6 | 19300977 | down | 48575 | RPL21P28 | AB− |
| SNP_A-8323763 | rs12010050 | X | 12888406 | up | 14739 | TMSL3 | AB+ |
| SNP_A-8323763 | rs12010050 | X | 12888406 | down | 37197 | TLR8 | AB+ |
| SNP_A-8380937 | rs9488847 | 6 | 116567513 | intron | 0 | NT5DC1 | AB+ |
| SNP_A-8494528 | rs1255575 | 14 | 62847659 | down | 1735 | GPHB5 | AB+ |
| SNP_A-8494528 | rs1255575 | 14 | 62847659 | down | 19347 | RHOJ | AB+ |
| SNP_A-2169771 | rs16895959 | 5 | 26719027 | down | 197438 | CDH9 | AB+/BB− |
| SNP_A-2169771 | rs16895959 | 5 | 26719027 | down | 769883 | MSN | AB+/BB− |
| SNP_A-8318466 | rs716682 | 5 | 57229214 | down | 556354 | PLK2 | AB+/BB− |
| SNP_A-8318466 | rs716682 | 5 | 57229214 | up | 414821 | ACTBL2 | AB+/BB− |
| SNP_A-1818056 | rs702743 | 5 | 154251584 | intron | 0 | GEMIN5 | BB− |
| SNP_A-8515705 | rs13355963 | 5 | 166265399 | up | 379021 | ODZ2 | BB− |
| SNP_A-8515705 | rs13355963 | 5 | 166265399 | up | 876485 | RPL7 | BB− |
| SNP_A-8515705 | rs13355963 | 5 | 166265399 | up | 1386270 | WWC1 | BB− |
| SNP_A-8619403 | rs16860657 | 3 | 149851458 | up | 46889 | AGTR1 | BB− |
| SNP_A-8619403 | rs16860657 | 3 | 149851458 | up | 483606 | HNRNPA1 | BB− |
| SNP_A-4250265 | rs2289507 | 3 | 101553906 | intron | 0 | NIT2 | BB+ |
| SNP_A-8415948 | rs17043069 | 2 | 113652278 | intron | 0 | PSD4 | BB+ |
| SNP_A-8460426 | rs7032233 | 9 | 3664809 | down | 149318 | GLIS3 | BB+ |
| SNP_A-8460426 | rs7032233 | 9 | 3664809 | up | 148826 | RFX3 | BB+ |

+= genotype at that SNP is longevity;
−= genotype at that SNP is deleterious.

Potential buffered deleterious genes were searched for by looking for those SNP markers whose genotype frequency showed a U-shape pattern, i.e. those showing a frequency drop as age increases in the controls but having a high frequency in the centenarians.

The combined control group (age between 43 and 94) and centenarian group, were again taken, a total of 615 subjects. For each SNP marker, genotypes that appear at least 10 times were taken. For each such genotype, the SNP marker was used as the dependent variable and fitted to two logistic regression models, one with age as the linear term, and the other with an additional quadratic term of age. The significance of the quadratic term is then determined by examining the difference of the deviance of the two models, which asymptotically follows a Chi-squared distribution with one degree of freedom. The statistical significance of the first model employing age as the linear term is similarly obtained by comparing its deviance with the null deviance. The computation is carried out using statistical software R, and its function glm.fit on a dual 4-core processor Mac Pro workstation, and is completed in less than 20 hours.

For a buffering gene to affect a deleterious gene, the allele frequency of the buffered deleterious gene should remain the same level among the subjects that carry the buffering gene. However, the allele frequency of the buffered deleterious gene should decrease among the subjects that do not carry the buffering gene as they age. Ideally the interaction between these two types of genes can be best detected on a sample in which the buffering genes (longevity genes) are enriched, for example, offspring of centenarians. For the genotyped control subjects, this interaction was studied. A significant interaction term implies that the genotype frequencies of the buffered gene changes differently between the two groups with and without the buffering gene.

When the presence of a genotype at one SNP is detected in a subject in combination with the presence of a genotype at another SNP, the combination of the genotypes at the two SNPs has a statistically significant correlation with the subject's likelihood of longevity (Tables 6, 9). A single combination may affect a single gene (Table 6) or more than one gene (Table 9). Characteristics of each SNP in the correlation are listed in Tables 7 and 8 for those correlations listed in Table 6, and Tables 10 and 11 for those correlations listed in Table 9.

The present invention is directed to the use of specific single nucleotide polymorphisms (SNPs) for determining an individual's likelihood of longevity or of developing age-related disease. The invention provides for genetic markers, which unlike any marker previously assessed, are enriched in subjects with exceptional longevity. Furthermore, better health and better cognitive function are reported in subjects who exhibit these markers, showing causation. Knowledge of the genetic markers will allow for the therapeutic intervention for those subjects genetically predisposed to age-related diseases in order to promote survival.

TABLE 6
Characteristics of Longevity Interactions

| Geno. | | | Chr.- | | Geno. | | | Chr.- | | |
|---|---|---|---|---|---|---|---|---|---|---|
| buffer- | | dbSNP- | Buffer- | Gene- | Buffer- | | dbSNP- | Buffer- | Gene- | |
| ing | Buffering | Buffering | ing | Buffering | ed | Buffered | Buffered | ed | Buffering | P-value |
| AA | SNP_A-8446732 | rs17154320 | 7 | CD36 | CC | SNP_A-8465134 | rs10278652 | 7 | SHH | 0.000005 |
| AA | SNP_A-8446732 | rs17154320 | 7 | CD36 | CC | SNP_A-8465134 | rs10278652 | 7 | tcag7.1213 | 0.000005 |
| AA | SNP_A-8446732 | rs17154320 | 7 | SEMA3C | CC | SNP_A-8465134 | rs10278652 | 7 | SHH | 0.000005 |
| AA | SNP_A-8446732 | rs17154320 | 7 | SEMA3C | CC | SNP_A-8465134 | rs10278652 | 7 | tcag7.1213 | 0.000005 |
| TT | SNP_A-1865739 | rs6094553 | 20 | EYA2 | CC | SNP_A-8465134 | rs10278652 | 7 | SHH | 0.000001 |
| AT | SNP_A-1865739 | rs6094553 | 20 | EYA2 | CC | SNP_A-8465134 | rs10278652 | 7 | SHH | 0.000001 |
| TT | SNP_A-1865739 | rs6094553 | 20 | EYA2 | CC | SNP_A-8465134 | rs10278652 | 7 | tcag7.1213 | 0.000001 |
| AT | SNP_A-1865739 | rs6094553 | 20 | EYA2 | CC | SNP_A-8465134 | rs10278652 | 7 | tcag7.1213 | 0.000001 |
| TT | SNP_A-2051880 | rs16951040 | 16 | TOX3 | CC | SNP_A-4296086 | rs1324362 | 1 | ROR1 | 0.000410 |
| AT | SNP_A-2051880 | rs16951040 | 16 | TOX3 | CC | SNP_A-4296086 | rs1324362 | 1 | ROR1 | 0.000410 |
| AA | SNP_A-2083501 | rs4686870 | 3 | MASP1 | AA | SNP_A-2294709 | rs3749796 | 5 | GFPT2 | 0.000024 |
| AA | SNP_A-2083501 | rs4686870 | 3 | MASP1 | AA | SNP_A-2294709 | rs3749796 | 5 | MAPK9 | 0.000024 |
| AA | SNP_A-2083501 | rs4686870 | 3 | MASP1 | AA | SNP_A-4218063 | rs10919593 | 1 | PTPRC | 0.000017 |
| AA | SNP_A-2083501 | rs4686870 | 3 | MASP1 | CC | SNP_A-4238494 | rs2862274 | 1 | FAM5B | 0.000694 |
| AA | SNP_A-2083501 | rs4686870 | 3 | MASP1 | CC | SNP_A-8301485 | rs16902320 | 8 | MYC | 0.000820 |
| AA | SNP_A-2083501 | rs4686870 | 3 | MASP1 | TT | SNP_A-2294709 | rs3749796 | 5 | GFPT2 | 0.000815 |
| AA | SNP_A-2083501 | rs4686870 | 3 | MASP1 | TT | SNP_A-2294709 | rs3749796 | 5 | MAPK9 | 0.000815 |
| AA | SNP_A-2083501 | rs4686870 | 3 | MASP1 | TT | SNP_A-4238494 | rs2862274 | 1 | FAM5B | 0.000020 |
| AA | SNP_A-2083501 | rs4686870 | 3 | RTP4 | AA | SNP_A-2294709 | rs3749796 | 5 | GFPT2 | 0.000024 |
| AA | SNP_A-2083501 | rs4686870 | 3 | RTP4 | AA | SNP_A-2294709 | rs3749796 | 5 | MAPK9 | 0.000024 |
| AA | SNP_A-2083501 | rs4686870 | 3 | RTP4 | AA | SNP_A-4218063 | rs10919593 | 1 | PTPRC | 0.000017 |
| AA | SNP_A-2083501 | rs4686870 | 3 | RTP4 | CC | SNP_A-4238494 | rs2862274 | 1 | FAM5B | 0.000694 |
| AA | SNP_A-2083501 | rs4686870 | 3 | RTP4 | CC | SNP_A-8301485 | rs16902320 | 8 | MYC | 0.000820 |
| AA | SNP_A-2083501 | rs4686870 | 3 | RTP4 | TT | SNP_A-2294709 | rs3749796 | 5 | GFPT2 | 0.000815 |
| AA | SNP_A-2083501 | rs4686870 | 3 | RTP4 | TT | SNP_A-2294709 | rs3749796 | 5 | MAPK9 | 0.000815 |
| AA | SNP_A-2083501 | rs4686870 | 3 | RTP4 | TT | SNP_A-4238494 | rs2862274 | 1 | FAM5B | 0.000020 |
| TT | SNP_A-2310343 | rs325380 | 15 | MEF2A | CC | SNP_A-8554692 | rs11030746 | 11 | METT5D1 | 0.000179 |
| AT | SNP_A-2310343 | rs325380 | 15 | MEF2A | CC | SNP_A-8554692 | rs11030746 | 11 | METT5D1 | 0.000179 |
| TT | SNP_A-2310343 | rs325380 | 15 | MEF2A | TT | SNP_A-2294709 | rs3749796 | 5 | GFPT2 | 0.000097 |
| AT | SNP_A-2310343 | rs325380 | 15 | MEF2A | TT | SNP_A-2294709 | rs3749796 | 5 | GFPT2 | 0.000097 |
| TT | SNP_A-2310343 | rs325380 | 15 | MEF2A | TT | SNP_A-2294709 | rs3749796 | 5 | MAPK9 | 0.000097 |
| AT | SNP_A-2310343 | rs325380 | 15 | MEF2A | TT | SNP_A-2294709 | rs3749796 | 5 | MAPK9 | 0.000097 |
| TT | SNP_A-2310343 | rs325380 | 15 | MEF2A | TT | SNP_A-8554692 | rs11030746 | 11 | METT5D1 | 0.000144 |
| AT | SNP_A-2310343 | rs325380 | 15 | MEF2A | TT | SNP_A-8554692 | rs11030746 | 11 | METT5D1 | 0.000144 |
| TT | SNP_A-4297368 | rs6851158 | 4 | ANK2 | GG | SNP_A-4282690 | rs17120428 | 1 | NFIA | 0.000676 |
| AT | SNP_A-4297368 | rs6851158 | 4 | ANK2 | GG | SNP_A-4282690 | rs17120428 | 1 | NFIA | 0.000676 |
| TT | SNP_A-8328958 | rs16880188 | 5 | LOC729506 | CC | SNP_A-4262900 | rs17003277 | 21 | SAMSN1 | 0.000181 |
| AT | SNP_A-8328958 | rs16880188 | 5 | LOC729506 | CC | SNP_A-4262900 | rs17003277 | 21 | SAMSN1 | 0.000181 |
| TT | SNP_A-8328958 | rs16880188 | 5 | LOC729506 | CC | SNP_A-8554692 | rs11030746 | 11 | METT5D1 | 0.000173 |
| AT | SNP_A-8328958 | rs16880188 | 5 | LOC729506 | CC | SNP_A-8554692 | rs11030746 | 11 | METT5D1 | 0.000173 |
| TT | SNP_A-8328958 | rs16880188 | 5 | LOC729506 | TT | SNP_A-4262900 | rs17003277 | 21 | SAMSN1 | 0.000138 |
| AT | SNP_A-8328958 | rs16880188 | 5 | LOC729506 | TT | SNP_A-4262900 | rs17003277 | 21 | SAMSN1 | 0.000138 |
| TT | SNP_A-8328958 | rs16880188 | 5 | LOC729506 | TT | SNP_A-8554692 | rs11030746 | 11 | METT5D1 | 0.000188 |
| AT | SNP_A-8328958 | rs16880188 | 5 | LOC729506 | TT | SNP_A-8554692 | rs11030746 | 11 | METT5D1 | 0.000188 |
| TT | SNP_A-8328958 | rs16880188 | 5 | MTRR | CC | SNP_A-4262900 | rs17003277 | 21 | SAMSN1 | 0.000181 |
| AT | SNP_A-8328958 | rs16880188 | 5 | MTRR | CC | SNP_A-4262900 | rs17003277 | 21 | SAMSN1 | 0.000181 |
| TT | SNP_A-8328958 | rs16880188 | 5 | MTRR | CC | SNP_A-8554692 | rs11030746 | 11 | METT5D1 | 0.000173 |
| AT | SNP_A-8328958 | rs16880188 | 5 | MTRR | CC | SNP_A-8554692 | rs11030746 | 11 | METT5D1 | 0.000173 |
| TT | SNP_A-8328958 | rs16880188 | 5 | MTRR | TT | SNP_A-4262900 | rs17003277 | 21 | SAMSN1 | 0.000138 |
| AT | SNP_A-8328958 | rs16880188 | 5 | MTRR | TT | SNP_A-4262900 | rs17003277 | 21 | SAMSN1 | 0.000138 |
| TT | SNP_A-8328958 | rs16880188 | 5 | MTRR | TT | SNP_A-8554692 | rs11030746 | 11 | METT5D1 | 0.000188 |
| AT | SNP_A-8328958 | rs16880188 | 5 | MTRR | TT | SNP_A-8554692 | rs11030746 | 11 | METT5D1 | 0.000188 |
| AA | SNP_A-8347387 | rs2525841 | 12 | ACSS3 | CT | SNP_A-8682320 | rs12838423 | X | NLGN4X | 0.000190 |
| AA | SNP_A-8347387 | rs2525841 | 12 | ACSS3 | GT | SNP_A-8456832 | rs12857956 | X | NLGN4X | 0.000190 |
| TT | SNP_A-8619237 | rs1822910 | 3 | GM2AP | GG | SNP_A-2267424 | rs5981167 | X | PJA1 | 0.000805 |
| AT | SNP_A-8619237 | rs1822910 | 3 | GM2AP | GG | SNP_A-2267424 | rs5981167 | X | PJA1 | 0.000805 |
| TT | SNP_A-8619237 | rs1822910 | 3 | GM2AP | GG | SNP_A-2267424 | rs5981167 | X | EFNB1 | 0.000805 |
| AT | SNP_A-8619237 | rs1822910 | 3 | GM2AP | GG | SNP_A-2267424 | rs5981167 | X | EFNB1 | 0.000805 |
| AA | SNP_A-8619277 | rs696405 | 3 | MASP1 | TT | SNP_A-4238494 | rs2862274 | 1 | FAM5B | 0.000417 |
| TT | SNP_A-8687186 | rs17014445 | 2 | MYADML | CC | SNP_A-1797721 | rs11183915 | 12 | LOC728148 | 0.000456 |
| AT | SNP_A-8687186 | rs17014445 | 2 | MYADML | CC | SNP_A-1797721 | rs11183915 | 12 | LOC728148 | 0.000456 |
| TT | SNP_A-8687186 | rs17014445 | 2 | MYADML | CC | SNP_A-4296112 | rs11103184 | 9 | KCNT1 | 0.000029 |
| AT | SNP_A-8687186 | rs17014445 | 2 | MYADML | CC | SNP_A-4296112 | rs11103184 | 9 | KCNT1 | 0.000029 |
| TT | SNP_A-8687186 | rs17014445 | 2 | MYADML | CC | SNP_A-8691130 | rs11103512 | 9 | COL5A1 | 0.000078 |
| AT | SNP_A-8687186 | rs17014445 | 2 | MYADML | CC | SNP_A-8691130 | rs11103512 | 9 | COL5A1 | 0.000078 |
| TT | SNP_A-8687186 | rs17014445 | 2 | MYADML | CT | SNP_A-8449633 | rs9586459 | 13 | DAOA | 0.000045 |
| AT | SNP_A-8687186 | rs17014445 | 2 | MYADML | CT | SNP_A-8449633 | rs9586459 | 13 | DAOA | 0.000045 |
| TT | SNP_A-8687186 | rs17014445 | 2 | MYADML | GG | SNP_A-4218063 | rs10919593 | 1 | PTPRC | 0.000807 |
| AT | SNP_A-8687186 | rs17014445 | 2 | MYADML | GG | SNP_A-4218063 | rs10919593 | 1 | PTPRC | 0.000807 |
| TT | SNP_A-8687186 | rs17014445 | 2 | MYADML | GG | SNP_A-4218063 | rs10919593 | 1 | RPS2P9 | 0.000807 |
| AT | SNP_A-8687186 | rs17014445 | 2 | MYADML | GG | SNP_A-4218063 | rs10919593 | 1 | RPS2P9 | 0.000807 |
| TT | SNP_A-8687186 | rs17014445 | 2 | MYADML | TT | SNP_A-8449633 | rs9586459 | 13 | DAOA | 0.000045 |
| AT | SNP_A-8687186 | rs17014445 | 2 | MYADML | TT | SNP_A-8449633 | rs9586459 | 13 | DAOA | 0.000045 |
| TT | SNP_A-8687186 | rs17014445 | 2 | MYADML | TT | SNP_A-8619237 | rs1822910 | 3 | RPL21P46 | 0.000745 |
| AT | SNP_A-8687186 | rs17014445 | 2 | MYADML | TT | SNP_A-8619237 | rs1822910 | 3 | RPL21P46 | 0.000745 |
| TT | SNP_A-8687186 | rs17014445 | 2 | LOC100130506 | CC | SNP_A-4296112 | rs11103184 | 9 | KCNT1 | 0.000029 |
| AT | SNP_A-8687186 | rs17014445 | 2 | LOC100130506 | CC | SNP_A-4296112 | rs11103184 | 9 | KCNT1 | 0.000029 |
| TT | SNP_A-8687186 | rs17014445 | 2 | LOC100130506 | GG | SNP_A-4218063 | rs10919593 | 1 | PTPRC | 0.000807 |
| AT | SNP_A-8687186 | rs17014445 | 2 | LOC100130506 | GG | SNP_A-4218063 | rs10919593 | 1 | PTPRC | 0.000807 |
| TT | SNP_A-8687186 | rs17014445 | 2 | SLC25A5 | CC | SNP_A-4296112 | rs11103184 | 9 | KCNT1 | 0.000029 |
| AT | SNP_A-8687186 | rs17014445 | 2 | SLC25A5 | CC | SNP_A-4296112 | rs11103184 | 9 | KCNT1 | 0.000029 |
| TT | SNP_A-8687186 | rs17014445 | 2 | SLC25A5 | GG | SNP_A-4218063 | rs10919593 | 1 | PTPRC | 0.000807 |
| AT | SNP_A-8687186 | rs17014445 | 2 | SLC25A5 | GG | SNP_A-4218063 | rs10919593 | 1 | PTPRC | 0.000807 |
| AA | SNP_A-8446732 | rs17154320 | 7 | CD36 | CC | SNP_A-8465134 | rs10278652 | 7 | SHH | 0.000004 |
| GG | SNP_A-8446732 | rs17154320 | 7 | CD36 | CC | SNP_A-8465134 | rs10278652 | 7 | SHH | 0.000004 |
| AA | SNP_A-8446732 | rs17154320 | 7 | CD36 | CC | SNP_A-8465134 | rs10278652 | 7 | tcag7.1213 | 0.000004 |
| GG | SNP_A-8446732 | rs17154320 | 7 | CD36 | CC | SNP_A-8465134 | rs10278652 | 7 | tcag7.1213 | 0.000004 |
| AA | SNP_A-8446732 | rs17154320 | 7 | SEMA3C | CC | SNP_A-8465134 | rs10278652 | 7 | SHH | 0.000004 |
| GG | SNP_A-8446732 | rs17154320 | 7 | SEMA3C | CC | SNP_A-8465134 | rs10278652 | 7 | SHH | 0.000004 |
| AA | SNP_A-8446732 | rs17154320 | 7 | SEMA3C | CC | SNP_A-8465134 | rs10278652 | 7 | tcag7.1213 | 0.000004 |
| GG | SNP_A-8446732 | rs17154320 | 7 | SEMA3C | CC | SNP_A-8465134 | rs10278652 | 7 | tcag7.1213 | 0.000004 |
| AA | SNP_A-4180725 | rs7106605 | 11 | DYNC2H1 | AA | SNP_A-2294709 | rs3749796 | 5 | GFPT2 | 0.000260 |
| GG | SNP_A-4180725 | rs7106605 | 11 | DYNC2H1 | AA | SNP_A-2294709 | rs3749796 | 5 | GFPT2 | 0.000260 |
| AA | SNP_A-4180725 | rs7106605 | 11 | DYNC2H1 | AA | SNP_A-2294709 | rs3749796 | 5 | MAPK9 | 0.000260 |
| GG | SNP_A-4180725 | rs7106605 | 11 | DYNC2H1 | AA | SNP_A-2294709 | rs3749796 | 5 | MAPK9 | 0.000260 |
| AA | SNP_A-4180725 | rs7106605 | 11 | PDGFD | AA | SNP_A-2294709 | rs3749796 | 5 | GFPT2 | 0.000260 |
| GG | SNP_A-4180725 | rs7106605 | 11 | PDGFD | AA | SNP_A-2294709 | rs3749796 | 5 | GFPT2 | 0.000260 |
| AA | SNP_A-4180725 | rs7106605 | 11 | PDGFD | AA | SNP_A-2294709 | rs3749796 | 5 | MAPK9 | 0.000260 |
| GG | SNP_A-4180725 | rs7106605 | 11 | PDGFD | AA | SNP_A-2294709 | rs3749796 | 5 | MAPK9 | 0.000260 |
| AG | SNP_A-4286901 | rs1528708 | 8 | UNC5D | GG | SNP_A-8478767 | rs4595891 | 18 | CDH19 | 0.000671 |
| AG | SNP_A-8282993 | rs2382993 | 8 | MTSS1 | CC | SNP_A-4296112 | rs11103184 | 9 | KCNT1 | 0.000452 |
| AG | SNP_A-8494528 | rs1255575 | 14 | GPHB5 | CC | SNP_A-4238494 | rs2862274 | 1 | FAM5B | 0.000932 |
| AG | SNP_A-8494528 | rs1255575 | 14 | GPHB5 | TT | SNP_A-4238494 | rs2862274 | 1 | FAM5B | 0.000015 |
| AG | SNP_A-8494528 | rs1255575 | 14 | RHOJ | CC | SNP_A-4238494 | rs2862274 | 1 | FAM5B | 0.000932 |
| AG | SNP_A-8494528 | rs1255575 | 14 | RHOJ | TT | SNP_A-4238494 | rs2862274 | 1 | FAM5B | 0.000015 |
| AA | SNP_A-8605897 | rs7130910 | 11 | DYNC2H1 | AA | SNP_A-2294709 | rs3749796 | 5 | GFPT2 | 0.000332 |
| GG | SNP_A-8605897 | rs7130910 | 11 | DYNC2H1 | AA | SNP_A-2294709 | rs3749796 | 5 | GFPT2 | 0.000332 |
| AA | SNP_A-8605897 | rs7130910 | 11 | DYNC2H1 | AA | SNP_A-2294709 | rs3749796 | 5 | MAPK9 | 0.000332 |
| GG | SNP_A-8605897 | rs7130910 | 11 | DYNC2H1 | AA | SNP_A-2294709 | rs3749796 | 5 | MAPK9 | 0.000332 |
| AA | SNP_A-8605897 | rs7130910 | 11 | PDGFD | AA | SNP_A-2294709 | rs3749796 | 5 | GFPT2 | 0.000332 |
| GG | SNP_A-8605897 | rs7130910 | 11 | PDGFD | AA | SNP_A-2294709 | rs3749796 | 5 | GFPT2 | 0.000332 |
| AA | SNP_A-8605897 | rs7130910 | 11 | PDGFD | AA | SNP_A-2294709 | rs3749796 | 5 | MAPK9 | 0.000332 |
| GG | SNP_A-8605897 | rs7130910 | 11 | PDGFD | AA | SNP_A-2294709 | rs3749796 | 5 | MAPK9 | 0.000332 |
| CC | SNP_A-1850528 | rs17049044 | 4 | PCDH18 | AA | SNP_A-8529547 | rs553369 | X | SPRY3 | 0.000930 |
| CC | SNP_A-1850528 | rs17049044 | 4 | PCDH18 | AA | SNP_A-8529547 | rs553369 | X | TMLHE | 0.000930 |
| GG | SNP_A-1905146 | rs2012129 | 15 | MEF2A | CC | SNP_A-8554692 | rs11030746 | 11 | METT5D1 | 0.000102 |
| CG | SNP_A-1905146 | rs2012129 | 15 | MEF2A | CC | SNP_A-8554692 | rs11030746 | 11 | METT5D1 | 0.000102 |
| GG | SNP_A-1905146 | rs2012129 | 15 | MEF2A | TT | SNP_A-2294709 | rs3749796 | 5 | GFPT2 | 0.000123 |
| CG | SNP_A-1905146 | rs2012129 | 15 | MEF2A | TT | SNP_A-2294709 | rs3749796 | 5 | GFPT2 | 0.000123 |
| GG | SNP_A-1905146 | rs2012129 | 15 | MEF2A | TT | SNP_A-2294709 | rs3749796 | 5 | MAPK9 | 0.000123 |
| CG | SNP_A-1905146 | rs2012129 | 15 | MEF2A | TT | SNP_A-2294709 | rs3749796 | 5 | MAPK9 | 0.000123 |
| GG | SNP_A-1905146 | rs2012129 | 15 | MEF2A | TT | SNP_A-8554692 | rs11030746 | 11 | METT5D1 | 0.000070 |
| CG | SNP_A-1905146 | rs2012129 | 15 | MEF2A | TT | SNP_A-8554692 | rs11030746 | 11 | METT5D1 | 0.000070 |
| GG | SNP_A-1935646 | rs2098846 | — | | CC | SNP_A-4262900 | rs17003277 | 21 | SAMSN1 | 0.000586 |
| CG | SNP_A-1935646 | rs2098846 | — | | CC | SNP_A-4262900 | rs17003277 | 21 | SAMSN1 | 0.000586 |
| GG | SNP_A-1935646 | rs2098846 | — | | TT | SNP_A-4262900 | rs17003277 | 21 | SAMSN1 | 0.000819 |
| CG | SNP_A-1935646 | rs2098846 | — | | TT | SNP_A-4262900 | rs17003277 | 21 | SAMSN1 | 0.000819 |
| CC | SNP_A-2051880 | rs16951040 | 16 | TOX3 | AG | SNP_A-8446732 | rs17154320 | 7 | SEMA3C | 0.000670 |
| GG | SNP_A-4289735 | rs2061007 | 15 | LYSMD4 | AA | SNP_A-8301485 | rs16902320 | 8 | MYC | 0.000364 |
| CG | SNP_A-4289735 | rs2061007 | 15 | LYSMD4 | AA | SNP_A-8301485 | rs16902320 | 8 | MYC | 0.000364 |
| GG | SNP_A-4289735 | rs2061007 | 15 | LYSMD4 | CC | SNP_A-4238494 | rs2862274 | 1 | FAM5B | 0.000265 |
| CG | SNP_A-4289735 | rs2061007 | 15 | LYSMD4 | CC | SNP_A-4238494 | rs2862274 | 1 | FAM5B | 0.000265 |
| GG | SNP_A-4289735 | rs2061007 | 15 | LYSMD4 | CC | SNP_A-4262900 | rs17003277 | 21 | SAMSN1 | 0.000060 |
| CG | SNP_A-4289735 | rs2061007 | 15 | LYSMD4 | CC | SNP_A-4262900 | rs17003277 | 21 | SAMSN1 | 0.000060 |
| GG | SNP_A-4289735 | rs2061007 | 15 | LYSMD4 | CC | SNP_A-8301485 | rs16902320 | 8 | MYC | 0.000213 |
| CG | SNP_A-4289735 | rs2061007 | 15 | LYSMD4 | CC | SNP_A-8301485 | rs16902320 | 8 | MYC | 0.000213 |
| GG | SNP_A-4289735 | rs2061007 | 15 | LYSMD4 | CC | SNP_A-8554692 | rs11030746 | 11 | METT5D1 | 0.000179 |
| CG | SNP_A-4289735 | rs2061007 | 15 | LYSMD4 | CC | SNP_A-8554692 | rs11030746 | 11 | METT5D1 | 0.000179 |
| GG | SNP_A-4289735 | rs2061007 | 15 | LYSMD4 | CC | SNP_A-8716016 | rs3816612 | 15 | ITGA11 | 0.000047 |
| CG | SNP_A-4289735 | rs2061007 | 15 | LYSMD4 | CC | SNP_A-8716016 | rs3816612 | 15 | ITGA11 | 0.000047 |
| GG | SNP_A-4289735 | rs2061007 | 15 | LYSMD4 | TT | SNP_A-2294709 | rs3749796 | 5 | GFPT2 | 0.000099 |
| CG | SNP_A-4289735 | rs2061007 | 15 | LYSMD4 | TT | SNP_A-2294709 | rs3749796 | 5 | GFPT2 | 0.000099 |
| GG | SNP_A-4289735 | rs2061007 | 15 | LYSMD4 | TT | SNP_A-2294709 | rs3749796 | 5 | MAPK9 | 0.000099 |
| CG | SNP_A-4289735 | rs2061007 | 15 | LYSMD4 | TT | SNP_A-2294709 | rs3749796 | 5 | MAPK9 | 0.000099 |
| GG | SNP_A-4289735 | rs2061007 | 15 | LYSMD4 | TT | SNP_A-4238494 | rs2862274 | 1 | FAM5B | 0.000247 |
| CG | SNP_A-4289735 | rs2061007 | 15 | LYSMD4 | TT | SNP_A-4238494 | rs2862274 | 1 | FAM5B | 0.000247 |
| GG | SNP_A-4289735 | rs2061007 | 15 | LYSMD4 | TT | SNP_A-8554692 | rs11030746 | 11 | METT5D1 | 0.000145 |
| CG | SNP_A-4289735 | rs2061007 | 15 | LYSMD4 | TT | SNP_A-8554692 | rs11030746 | 11 | METT5D1 | 0.000145 |
| GG | SNP_A-4289735 | rs2061007 | 15 | LYSMD4 | TT | SNP_A-8619237 | rs1822910 | 3 | RPL21P46 | 0.000000 |
| CG | SNP_A-4289735 | rs2061007 | 15 | LYSMD4 | TT | SNP_A-8619237 | rs1822910 | 3 | RPL21P46 | 0.000000 |
| GG | SNP_A-4289735 | rs2061007 | 15 | LYSMD4 | TT | SNP_A-8716016 | rs3816612 | 15 | ITGA11 | 0.000381 |
| CG | SNP_A-4289735 | rs2061007 | 15 | LYSMD4 | TT | SNP_A-8716016 | rs3816612 | 15 | ITGA11 | 0.000381 |
| CC | SNP_A-8300818 | rs2274703 | 1 | LEMD1 | CC | SNP_A-8475711 | rs7827228 | 8 | CSMD1 | 0.000490 |
| CC | SNP_A-8300818 | rs2274703 | 1 | LEMD1 | CC | SNP_A-8475711 | rs7827228 | 8 | MYOM2 | 0.000490 |
| CC | SNP_A-8300818 | rs2274703 | 1 | LEMD1 | CG | SNP_A-1881982 | rs2237342 | 7 | TAX1BP1 | 0.000234 |
| GG | SNP_A-8374392 | rs6066159 | 20 | EYA2 | AA | SNP_A-4218063 | rs10919593 | 1 | PTPRC | 0.000576 |
| CG | SNP_A-8374392 | rs6066159 | 20 | EYA2 | AA | SNP_A-4218063 | rs10919593 | 1 | PTPRC | 0.000576 |
| GG | SNP_A-8374392 | rs6066159 | 20 | EYA2 | AA | SNP_A-8636331 | rs10920481 | 1 | KDM5B | 0.000007 |
| CG | SNP_A-8374392 | rs6066159 | 20 | EYA2 | AA | SNP_A-8636331 | rs10920481 | 1 | KDM5B | 0.000007 |
| GG | SNP_A-8374392 | rs6066159 | 20 | EYA2 | CC | SNP_A-8465134 | rs10278652 | 7 | SHH | 0.000001 |
| CG | SNP_A-8374392 | rs6066159 | 20 | EYA2 | CC | SNP_A-8465134 | rs10278652 | 7 | SHH | 0.000001 |
| GG | SNP_A-8374392 | rs6066159 | 20 | EYA2 | CC | SNP_A-8465134 | rs10278652 | 7 | tcag7.1213 | 0.000001 |
| CG | SNP_A-8374392 | rs6066159 | 20 | EYA2 | CC | SNP_A-8465134 | rs10278652 | 7 | tcag7.1213 | 0.000001 |
| CC | SNP_A-8452641 | rs1499953 | 3 | GAP43 | AA | SNP_A-4218063 | rs10919593 | 1 | PTPRC | 0.000058 |
| CC | SNP_A-8452641 | rs1499953 | 3 | ZBTB20 | AA | SNP_A-4218063 | rs10919593 | 1 | PTPRC | 0.000058 |
| CC | SNP_A-8639378 | rs12432648 | 14 | LRFN5 | CT | SNP_A-8682320 | rs12838423 | X | NLGN4X | 0.000208 |
| CC | SNP_A-8639378 | rs12432648 | 14 | LRFN5 | GT | SNP_A-8456832 | rs12857956 | X | NLGN4X | 0.000208 |
| CG | SNP_A-8451881 | rs10041500 | 5 | FLJ25076 | AG | SNP_A-8548351 | rs5912192 | X | P2RY10 | 0.000004 |
| CG | SNP_A-8451881 | rs10041500 | 5 | FLJ25076 | AG | SNP_A-8548351 | rs5912192 | X | LPAR4 | 0.000004 |
| CG | SNP_A-8451881 | rs10041500 | 5 | MED10 | AG | SNP_A-8548351 | rs5912192 | X | P2RY10 | 0.000004 |
| CG | SNP_A-8451881 | rs10041500 | 5 | MED10 | AG | SNP_A-8548351 | rs5912192 | X | LPAR4 | 0.000004 |
| CC | SNP_A-8639378 | rs12432648 | 14 | LRFN5 | CT | SNP_A-8682320 | rs12838423 | X | NLGN4X | 0.000208 |
| GG | SNP_A-8639378 | rs12432648 | 14 | LRFN5 | CT | SNP_A-8682320 | rs12838423 | X | NLGN4X | 0.000208 |
| CC | SNP_A-8639378 | rs12432648 | 14 | LRFN5 | GT | SNP_A-8456832 | rs12857956 | X | NLGN4X | 0.000208 |
| GG | SNP_A-8639378 | rs12432648 | 14 | LRFN5 | GT | SNP_A-8456832 | rs12857956 | X | NLGN4X | 0.000208 |
| CT | SNP_A-2032934 | rs17775764 | 5 | FLJ25076 | AG | SNP_A-8548351 | rs5912192 | X | P2RY10 | 0.000004 |
| CT | SNP_A-2032934 | rs17775764 | 5 | FLJ25076 | AG | SNP_A-8548351 | rs5912192 | X | LPAR4 | 0.000004 |
| CT | SNP_A-2032934 | rs17775764 | 5 | MED10 | AG | SNP_A-8548351 | rs5912192 | X | P2RY10 | 0.000004 |
| CT | SNP_A-2032934 | rs17775764 | 5 | MED10 | AG | SNP_A-8548351 | rs5912192 | X | LPAR4 | 0.000004 |
| CT | SNP_A-4291022 | rs5979604 | X | FRMPD4 | AA | SNP_A-2294709 | rs3749796 | 5 | GFPT2 | 0.000867 |
| CT | SNP_A-4291022 | rs5979604 | X | FRMPD4 | AA | SNP_A-2294709 | rs3749796 | 5 | MAPK9 | 0.000867 |
| CC | SNP_A-8479146 | rs7153013 | 14 | C14orf145 | TT | SNP_A-4262900 | rs17003277 | 21 | SAMSN1 | 0.000619 |
| TT | SNP_A-8479146 | rs7153013 | 14 | C14orf145 | TT | SNP_A-4262900 | rs17003277 | 21 | SAMSN1 | 0.000619 |
| CC | SNP_A-8479146 | rs7153013 | 14 | DIO2 | TT | SNP_A-4262900 | rs17003277 | 21 | SAMSN1 | 0.000619 |
| TT | SNP_A-8479146 | rs7153013 | 14 | DIO2 | TT | SNP_A-4262900 | rs17003277 | 21 | SAMSN1 | 0.000619 |
| CT | SNP_A-8665678 | rs11738547 | 5 | FAM173B | AA | SNP_A-2294709 | rs3749796 | 5 | GFPT2 | 0.000312 |
| CT | SNP_A-8665678 | rs11738547 | 5 | FAM173B | AA | SNP_A-2294709 | rs3749796 | 5 | MAPK9 | 0.000312 |
| CT | SNP_A-8665678 | rs11738547 | 5 | FAM173B | AA | SNP_A-8299128 | rs1257689 | 14 | BCL11B | 0.000765 |
| CT | SNP_A-8665678 | rs11738547 | 5 | FAM173B | CC | SNP_A-8554692 | rs11030746 | 11 | METT5D1 | 0.000423 |
| CT | SNP_A-8665678 | rs11738547 | 5 | FAM173B | GG | SNP_A-4282690 | rs17120428 | 1 | NFIA | 0.000487 |
| CT | SNP_A-8665678 | rs11738547 | 5 | FAM173B | TT | SNP_A-2294709 | rs3749796 | 5 | GFPT2 | 0.000211 |
| CT | SNP_A-8665678 | rs11738547 | 5 | FAM173B | TT | SNP_A-2294709 | rs3749796 | 5 | MAPK9 | 0.000211 |
| CT | SNP_A-8665678 | rs11738547 | 5 | FAM173B | TT | SNP_A-8554692 | rs11030746 | 11 | METT5D1 | 0.000472 |
| CT | SNP_A-8665678 | rs11738547 | 5 | TAS2R1 | AA | SNP_A-2294709 | rs3749796 | 5 | GFPT2 | 0.000312 |
| CT | SNP_A-8665678 | rs11738547 | 5 | TAS2R1 | AA | SNP_A-2294709 | rs3749796 | 5 | MAPK9 | 0.000312 |
| CT | SNP_A-8665678 | rs11738547 | 5 | TAS2R1 | AA | SNP_A-8299128 | rs1257689 | 14 | BCL11B | 0.000765 |
| CT | SNP_A-8665678 | rs11738547 | 5 | TAS2R1 | CC | SNP_A-8554692 | rs11030746 | 11 | METT5D1 | 0.000423 |
| CT | SNP_A-8665678 | rs11738547 | 5 | TAS2R1 | GG | SNP_A-4282690 | rs17120428 | 1 | NFIA | 0.000487 |
| CT | SNP_A-8665678 | rs11738547 | 5 | TAS2R1 | TT | SNP_A-2294709 | rs3749796 | 5 | GFPT2 | 0.000211 |
| CT | SNP_A-8665678 | rs11738547 | 5 | TAS2R1 | TT | SNP_A-2294709 | rs3749796 | 5 | MAPK9 | 0.000211 |
| CT | SNP_A-8665678 | rs11738547 | 5 | TAS2R1 | TT | SNP_A-8554692 | rs11030746 | 11 | METT5D1 | 0.000472 |
| GG | SNP_A-1854119 | rs7221263 | 17 | PPY | AA | SNP_A-2294709 | rs3749796 | 5 | GFPT2 | 0.000061 |
| GG | SNP_A-1854119 | rs7221263 | 17 | PPY | AA | SNP_A-2294709 | rs3749796 | 5 | MAPK9 | 0.000061 |
| GG | SNP_A-1854119 | rs7221263 | 17 | PPY | AA | SNP_A-4282690 | rs17120428 | 1 | NFIA | 0.000657 |
| GG | SNP_A-1854119 | rs7221263 | 17 | PPY | AA | SNP_A-8301485 | rs16902320 | 8 | MYC | 0.000924 |
| GG | SNP_A-1854119 | rs7221263 | 17 | PPY | CC | SNP_A-4238494 | rs2862274 | 1 | FAM5B | 0.000082 |
| GG | SNP_A-1854119 | rs7221263 | 17 | PPY | CC | SNP_A-4262900 | rs17003277 | 21 | SAMSN1 | 0.000090 |
| GG | SNP_A-1854119 | rs7221263 | 17 | PPY | CC | SNP_A-4296112 | rs11103184 | 9 | KCNT1 | 0.000194 |
| GG | SNP_A-1854119 | rs7221263 | 17 | PPY | CC | SNP_A-8554692 | rs11030746 | 11 | METT5D1 | 0.000020 |
| GG | SNP_A-1854119 | rs7221263 | 17 | PPY | CC | SNP_A-8592516 | rs17078925 | 6 | UST | 0.000382 |
| GG | SNP_A-1854119 | rs7221263 | 17 | PPY | CC | SNP_A-8716016 | rs3816612 | 15 | ITGA11 | 0.000122 |
| GG | SNP_A-1854119 | rs7221263 | 17 | PPY | GG | SNP_A-4282690 | rs17120428 | 1 | NFIA | 0.000317 |
| GG | SNP_A-1854119 | rs7221263 | 17 | PPY | GG | SNP_A-8478767 | rs4595891 | 18 | CDH19 | 0.000272 |
| GG | SNP_A-1854119 | rs7221263 | 17 | PPY | TT | SNP_A-2294709 | rs3749796 | 5 | GFPT2 | 0.000030 |
| GG | SNP_A-1854119 | rs7221263 | 17 | PPY | TT | SNP_A-2294709 | rs3749796 | 5 | MAPK9 | 0.000030 |
| GG | SNP_A-1854119 | rs7221263 | 17 | PPY | TT | SNP_A-4238494 | rs2862274 | 1 | FAM5B | 0.000075 |
| GG | SNP_A-1854119 | rs7221263 | 17 | PPY | TT | SNP_A-4262900 | rs17003277 | 21 | SAMSN1 | 0.000075 |
| GG | SNP_A-1854119 | rs7221263 | 17 | PPY | TT | SNP_A-4296112 | rs11103184 | 9 | KCNT1 | 0.000285 |
| GG | SNP_A-1854119 | rs7221263 | 17 | PPY | TT | SNP_A-8478767 | rs4595891 | 18 | CDH19 | 0.000365 |
| GG | SNP_A-1854119 | rs7221263 | 17 | PPY | TT | SNP_A-8554692 | rs11030746 | 11 | METT5D1 | 0.000021 |
| GG | SNP_A-1854119 | rs7221263 | 17 | PPY | TT | SNP_A-8592516 | rs17078925 | 6 | UST | 0.000576 |
| GG | SNP_A-1854119 | rs7221263 | 17 | PPY | TT | SNP_A-8716016 | rs3816612 | 15 | ITGA11 | 0.000103 |
| CC | SNP_A-1996154 | rs10738555 | 9 | MLLT3 | GG | SNP_A-4218063 | rs10919593 | 1 | PTPRC | 0.000690 |
| GC | SNP_A-1996154 | rs10738555 | 9 | MLLT3 | GG | SNP_A-4218063 | rs10919593 | 1 | PTPRC | 0.000690 |
| CC | SNP_A-1996154 | rs10738555 | 9 | SLC24A2 | GG | SNP_A-4218063 | rs10919593 | 1 | PTPRC | 0.000690 |
| GC | SNP_A-1996154 | rs10738555 | 9 | SLC24A2 | GG | SNP_A-4218063 | rs10919593 | 1 | PTPRC | 0.000690 |
| GG | SNP_A-4272472 | rs1461809 | 3 | LOC645206 | TT | SNP_A-8716016 | rs3816612 | 15 | ITGA11 | 0.000073 |
| CC | SNP_A-8347387 | rs2525841 | 12 | ACSS3 | AG | SNP_A-2267424 | rs5981167 | X | PJA1 | 0.000738 |
| CG | SNP_A-8347387 | rs2525841 | 12 | ACSS3 | AG | SNP_A-2267424 | rs5981167 | X | PJA1 | 0.000738 |
| CC | SNP_A-8347387 | rs2525841 | 12 | ACSS3 | AG | SNP_A-2267424 | rs5981167 | X | EFNB1 | 0.000738 |
| CG | SNP_A-8347387 | rs2525841 | 12 | ACSS3 | AG | SNP_A-2267424 | rs5981167 | X | EFNB1 | 0.000738 |
| CC | SNP_A-8347387 | rs2525841 | 12 | ACSS3 | GG | SNP_A-2267424 | rs5981167 | X | PJA1 | 0.000665 |
| CG | SNP_A-8347387 | rs2525841 | 12 | ACSS3 | GG | SNP_A-2267424 | rs5981167 | X | PJA1 | 0.000665 |
| CC | SNP_A-8347387 | rs2525841 | 12 | ACSS3 | GG | SNP_A-2267424 | rs5981167 | X | EFNB1 | 0.000665 |
| CG | SNP_A-8347387 | rs2525841 | 12 | ACSS3 | GG | SNP_A-2267424 | rs5981167 | X | EFNB1 | 0.000665 |
| CC | SNP_A-8384021 | rs368866 | X | CPXCR1 | CC | SNP_A-4262900 | rs17003277 | 21 | SAMSN1 | 0.000702 |
| CG | SNP_A-8384021 | rs368866 | X | CPXCR1 | CC | SNP_A-4262900 | rs17003277 | 21 | SAMSN1 | 0.000702 |
| CC | SNP_A-8384021 | rs368866 | X | CPXCR1 | TT | SNP_A-4262900 | rs17003277 | 21 | SAMSN1 | 0.000852 |
| CG | SNP_A-8384021 | rs368866 | X | CPXCR1 | TT | SNP_A-4262900 | rs17003277 | 21 | SAMSN1 | 0.000852 |
| CC | SNP_A-8384021 | rs368866 | X | TGIF2LY | CC | SNP_A-4262900 | rs17003277 | 21 | SAMSN1 | 0.000702 |
| CG | SNP_A-8384021 | rs368866 | X | TGIF2LY | CC | SNP_A-4262900 | rs17003277 | 21 | SAMSN1 | 0.000702 |
| CC | SNP_A-8384021 | rs368866 | X | TGIF2LY | TT | SNP_A-4262900 | rs17003277 | 21 | SAMSN1 | 0.000852 |
| CG | SNP_A-8384021 | rs368866 | X | TGIF2LY | TT | SNP_A-4262900 | rs17003277 | 21 | SAMSN1 | 0.000852 |
| CC | SNP_A-8484091 | rs12348066 | 9 | PTPRD | CC | SNP_A-8717502 | rs536789 | X | P2RY10 | 0.000006 |
| CG | SNP_A-8484091 | rs12348066 | 9 | PTPRD | CC | SNP_A-8717502 | rs536789 | X | P2RY10 | 0.000006 |
| CC | SNP_A-8484091 | rs12348066 | 9 | PTPRD | CC | SNP_A-8717502 | rs536789 | X | LPAR4 | 0.000006 |
| CG | SNP_A-8484091 | rs12348066 | 9 | PTPRD | CC | SNP_A-8717502 | rs536789 | X | LPAR4 | 0.000006 |
| CC | SNP_A-8484091 | rs12348066 | 9 | PTPRD | GG | SNP_A-8281027 | rs496666 | X | P2RY10 | 0.000005 |
| CG | SNP_A-8484091 | rs12348066 | 9 | PTPRD | GG | SNP_A-8281027 | rs496666 | X | P2RY10 | 0.000005 |
| CC | SNP_A-8484091 | rs12348066 | 9 | PTPRD | GG | SNP_A-8281027 | rs496666 | X | LPAR4 | 0.000005 |
| CG | SNP_A-8484091 | rs12348066 | 9 | PTPRD | GG | SNP_A-8281027 | rs496666 | X | LPAR4 | 0.000005 |
| CC | SNP_A-8484091 | rs12348066 | 9 | PTPRD | GG | SNP_A-8548351 | rs5912192 | X | P2RY10 | 0.000009 |
| CG | SNP_A-8484091 | rs12348066 | 9 | PTPRD | GG | SNP_A-8548351 | rs5912192 | X | P2RY10 | 0.000009 |
| CC | SNP_A-8484091 | rs12348066 | 9 | PTPRD | GG | SNP_A-8548351 | rs5912192 | X | LPAR4 | 0.000009 |
| CG | SNP_A-8484091 | rs12348066 | 9 | PTPRD | GG | SNP_A-8548351 | rs5912192 | X | LPAR4 | 0.000009 |
| CC | SNP_A-8484091 | rs12348066 | 9 | PTPRD | TT | SNP_A-8546758 | rs7063827 | X | GPR174 | 0.000006 |
| CG | SNP_A-8484091 | rs12348066 | 9 | PTPRD | TT | SNP_A-8546758 | rs7063827 | X | GPR174 | 0.000006 |
| CC | SNP_A-8484091 | rs12348066 | 9 | PTPRD | TT | SNP_A-8546758 | rs7063827 | X | P2RY10 | 0.000006 |
| CG | SNP_A-8484091 | rs12348066 | 9 | PTPRD | TT | SNP_A-8546758 | rs7063827 | X | P2RY10 | 0.000006 |
| CC | SNP_A-8494528 | rs1255575 | 14 | GPHB5 | CC | SNP_A-4238494 | rs2862274 | 1 | FAM5B | 0.000932 |
| CG | SNP_A-8494528 | rs1255575 | 14 | GPHB5 | CC | SNP_A-4238494 | rs2862274 | 1 | FAM5B | 0.000932 |
| CC | SNP_A-8494528 | rs1255575 | 14 | GPHB5 | TT | SNP_A-4238494 | rs2862274 | 1 | FAM5B | 0.000015 |
| CG | SNP_A-8494528 | rs1255575 | 14 | GPHB5 | TT | SNP_A-4238494 | rs2862274 | 1 | FAM5B | 0.000015 |
| CC | SNP_A-8494528 | rs1255575 | 14 | RHOJ | CC | SNP_A-4238494 | rs2862274 | 1 | FAM5B | 0.000932 |
| CG | SNP_A-8494528 | rs1255575 | 14 | RHOJ | CC | SNP_A-4238494 | rs2862274 | 1 | FAM5B | 0.000932 |
| CC | SNP_A-8494528 | rs1255575 | 14 | RHOJ | TT | SNP_A-4238494 | rs2862274 | 1 | FAM5B | 0.000015 |
| CG | SNP_A-8494528 | rs1255575 | 14 | RHOJ | TT | SNP_A-4238494 | rs2862274 | 1 | FAM5B | 0.000015 |
| GG | SNP_A-8574340 | rs7579939 | 2 | ASB3 | AA | SNP_A-2294709 | rs3749796 | 5 | GFPT2 | 0.000133 |
| GG | SNP_A-8574340 | rs7579939 | 2 | ASB3 | AA | SNP_A-2294709 | rs3749796 | 5 | MAPK9 | 0.000133 |
| GG | SNP_A-8574340 | rs7579939 | 2 | NRXN1 | AA | SNP_A-2294709 | rs3749796 | 5 | GFPT2 | 0.000133 |
| GG | SNP_A-8574340 | rs7579939 | 2 | NRXN1 | AA | SNP_A-2294709 | rs3749796 | 5 | MAPK9 | 0.000133 |
| GG | SNP_A-8580357 | rs1430707 | 2 | FAM123C | AA | SNP_A-2294709 | rs3749796 | 5 | GFPT2 | 0.000154 |
| GG | SNP_A-8580357 | rs1430707 | 2 | FAM123C | AA | SNP_A-2294709 | rs3749796 | 5 | MAPK9 | 0.000154 |
| GG | SNP_A-8580357 | rs1430707 | 2 | FAM123C | CC | SNP_A-4238494 | rs2862274 | 1 | FAM5B | 0.000036 |
| GG | SNP_A-8580357 | rs1430707 | 2 | FAM123C | TT | SNP_A-4238494 | rs2862274 | 1 | FAM5B | 0.000033 |
| GG | SNP_A-8580357 | rs1430707 | 2 | FAM123C | TT | SNP_A-8300818 | rs2274703 | 1 | LEMD1 | 0.000007 |
| GG | SNP_A-8580357 | rs1430707 | 2 | LOC730032 | AA | SNP_A-2294709 | rs3749796 | 5 | GFPT2 | 0.000154 |
| GG | SNP_A-8580357 | rs1430707 | 2 | LOC730032 | AA | SNP_A-2294709 | rs3749796 | 5 | MAPK9 | 0.000154 |
| GG | SNP_A-8580357 | rs1430707 | 2 | LOC730032 | CC | SNP_A-4238494 | rs2862274 | 1 | FAM5B | 0.000036 |
| GG | SNP_A-8580357 | rs1430707 | 2 | LOC730032 | TT | SNP_A-4238494 | rs2862274 | 1 | FAM5B | 0.000033 |
| GG | SNP_A-8687186 | rs17014445 | 2 | MYADML | AA | SNP_A-2107652 | rs3925518 | 5 | CLK4 | 0.000666 |
| GG | SNP_A-8687186 | rs17014445 | 2 | MYADML | AA | SNP_A-2107652 | rs3925518 | 5 | ZNF354A | 0.000666 |
| GG | SNP_A-8687186 | rs17014445 | 2 | MYADML | AA | SNP_A-2294709 | rs3749796 | 5 | MAPK9 | 0.000361 |
| GG | SNP_A-8687186 | rs17014445 | 2 | MYADML | AA | SNP_A-2294709 | rs3749796 | 5 | GFPT2 | 0.000361 |
| GG | SNP_A-8687186 | rs17014445 | 2 | MYADML | AA | SNP_A-4218063 | rs10919593 | 1 | PTPRC | 0.000138 |
| GG | SNP_A-8687186 | rs17014445 | 2 | MYADML | AA | SNP_A-4218063 | rs10919593 | 1 | RPS2P9 | 0.000138 |
| GG | SNP_A-8687186 | rs17014445 | 2 | MYADML | AA | SNP_A-8347387 | rs2525841 | 12 | ACSS3 | 0.000481 |
| GG | SNP_A-8687186 | rs17014445 | 2 | MYADML | AG | SNP_A-8446732 | rs17154320 | 7 | SEMA3C | 0.000292 |
| GG | SNP_A-8687186 | rs17014445 | 2 | MYADML | AT | SNP_A-8703223 | rs6641231 | X | RPL7P57 | 0.000280 |
| GG | SNP_A-8687186 | rs17014445 | 2 | MYADML | AT | SNP_A-8703223 | rs6641231 | X | HAX1 | 0.000280 |
| GG | SNP_A-8687186 | rs17014445 | 2 | MYADML | CC | SNP_A-1797721 | rs11183915 | 12 | LOC728148 | 0.000474 |
| GG | SNP_A-8687186 | rs17014445 | 2 | MYADML | CC | SNP_A-4296112 | rs11103184 | 9 | KCNT1 | 0.000102 |
| GG | SNP_A-8687186 | rs17014445 | 2 | MYADML | CC | SNP_A-8475711 | rs7827228 | 8 | CSMD1 | 0.000777 |
| GG | SNP_A-8687186 | rs17014445 | 2 | MYADML | CC | SNP_A-8475711 | rs7827228 | 8 | MYOM2 | 0.000777 |
| GG | SNP_A-8687186 | rs17014445 | 2 | MYADML | CC | SNP_A-8691130 | rs11103512 | 9 | COL5A1 | 0.000059 |
| GG | SNP_A-8687186 | rs17014445 | 2 | MYADML | CG | SNP_A-8339688 | rs6540375 | X | RPL7P57 | 0.000485 |
| GG | SNP_A-8687186 | rs17014445 | 2 | MYADML | CG | SNP_A-8339688 | rs6540375 | X | AFF2 | 0.000485 |
| GG | SNP_A-8687186 | rs17014445 | 2 | MYADML | CT | SNP_A-8339687 | rs6419449 | X | RPL7P57 | 0.000527 |
| GG | SNP_A-8687186 | rs17014445 | 2 | MYADML | CT | SNP_A-8339687 | rs6419449 | X | AFF2 | 0.000527 |
| GG | SNP_A-8687186 | rs17014445 | 2 | MYADML | CT | SNP_A-8449633 | rs9586459 | 13 | DAOA | 0.000007 |
| GG | SNP_A-8687186 | rs17014445 | 2 | MYADML | GG | SNP_A-4218063 | rs10919593 | 1 | PTPRC | 0.000510 |
| GG | SNP_A-8687186 | rs17014445 | 2 | MYADML | GG | SNP_A-4218063 | rs10919593 | 1 | RPS2P9 | 0.000510 |
| GG | SNP_A-8687186 | rs17014445 | 2 | MYADML | TT | SNP_A-8449633 | rs9586459 | 13 | DAOA | 0.000007 |
| GG | SNP_A-8687186 | rs17014445 | 2 | MYADML | TT | SNP_A-8494779 | rs6077004 | 20 | LOC728383 | 0.000170 |
| GG | SNP_A-8687186 | rs17014445 | 2 | MYADML | TT | SNP_A-8494779 | rs6077004 | 20 | TARDBPL | 0.000170 |
| GG | SNP_A-8687186 | rs17014445 | 2 | MYADML | TT | SNP_A-8619237 | rs1822910 | 3 | RPL21P46 | 0.000214 |
| GG | SNP_A-8687186 | rs17014445 | 2 | LOC100130506 | AA | SNP_A-2294709 | rs3749796 | 5 | GFPT2 | 0.000361 |
| GG | SNP_A-8687186 | rs17014445 | 2 | LOC100130506 | AA | SNP_A-2294709 | rs3749796 | 5 | MAPK9 | 0.000361 |
| GG | SNP_A-8687186 | rs17014445 | 2 | LOC100130506 | AA | SNP_A-4218063 | rs10919593 | 1 | PTPRC | 0.000138 |
| GG | SNP_A-8687186 | rs17014445 | 2 | LOC100130506 | CC | SNP_A-4296112 | rs11103184 | 9 | KCNT1 | 0.000102 |
| GG | SNP_A-8687186 | rs17014445 | 2 | LOC100130506 | GG | SNP_A-4218063 | rs10919593 | 1 | PTPRC | 0.000510 |
| GG | SNP_A-8687186 | rs17014445 | 2 | SLC25A5 | AA | SNP_A-2294709 | rs3749796 | 5 | GFPT2 | 0.000361 |
| GG | SNP_A-8687186 | rs17014445 | 2 | SLC25A5 | AA | SNP_A-2294709 | rs3749796 | 5 | MAPK9 | 0.000361 |
| GG | SNP_A-8687186 | rs17014445 | 2 | SLC25A5 | AA | SNP_A-4218063 | rs10919593 | 1 | PTPRC | 0.000138 |
| GG | SNP_A-8687186 | rs17014445 | 2 | SLC25A5 | CC | SNP_A-4296112 | rs11103184 | 9 | KCNT1 | 0.000102 |
| GG | SNP_A-8687186 | rs17014445 | 2 | SLC25A5 | CT | SNP_A-8449633 | rs9586459 | 13 | DAOA | 0.000007 |
| GG | SNP_A-8687186 | rs17014445 | 2 | SLC25A5 | GG | SNP_A-4218063 | rs10919593 | 1 | PTPRC | 0.000510 |
| GG | SNP_A-8687186 | rs17014445 | 2 | SLC25A5 | TT | SNP_A-8449633 | rs9586459 | 13 | DAOA | 0.000007 |
| TT | SNP_A-8438227 | rs4686457 | 3 | MASP1 | AA | SNP_A-4218063 | rs10919593 | 1 | PTPRC | 0.000005 |
| TT | SNP_A-8438227 | rs4686457 | 3 | MASP1 | AA | SNP_A-4218063 | rs10919593 | 1 | RPS2P9 | 0.000005 |
| TT | SNP_A-8438227 | rs4686457 | 3 | MASP1 | TT | SNP_A-4238494 | rs2862274 | 1 | LOC400796 | 0.000007 |
| TT | SNP_A-8438227 | rs4686457 | 3 | RTP4 | AA | SNP_A-4218063 | rs10919593 | 1 | PTPRC | 0.000005 |
| TT | SNP_A-8438227 | rs4686457 | 3 | RTP4 | AA | SNP_A-4218063 | rs10919593 | 1 | RPS2P9 | 0.000005 |
| TT | SNP_A-8438227 | rs4686457 | 3 | RTP4 | TT | SNP_A-4238494 | rs2862274 | 1 | LOC400796 | 0.000007 |
| TT | SNP_A-8673617 | rs2615903 | 11 | HIPK3 | AA | SNP_A-2294709 | rs3749796 | 5 | MAPK9 | 0.000966 |
| TT | SNP_A-8673617 | rs2615903 | 11 | HIPK3 | AA | SNP_A-2294709 | rs3749796 | 5 | GFPT2 | 0.000966 |
| AA | SNP_A-1850528 | rs17049044 | 4 | PCDH18 | CT | SNP_A-1998103 | rs3859965 | X | NLGN4X | 0.000500 |
| AT | SNP_A-1850528 | rs17049044 | 4 | PCDH18 | CT | SNP_A-1998103 | rs3859965 | X | NLGN4X | 0.000500 |
| TT | SNP_A-2298975 | rs12276840 | 11 | OPCML | AC | SNP_A-8672156 | rs10227794 | 7 | POM121L12 | 0.000009 |
| TT | SNP_A-2298975 | rs12276840 | 11 | OPCML | CC | SNP_A-8672156 | rs10227794 | 7 | POM121L12 | 0.000009 |
| AA | SNP_A-8369202 | rs533391 | 13 | HS6ST3 | CG | SNP_A-1881982 | rs2237342 | 7 | TAX1BP1 | 0.000663 |
| AT | SNP_A-8369202 | rs533391 | 13 | HS6ST3 | CG | SNP_A-1881982 | rs2237342 | 7 | TAX1BP1 | 0.000663 |
| AA | SNP_A-8419894 | rs17083201 | 9 | TLE1 | GT | SNP_A-1831539 | rs2144497 | X | DMD | 0.000000 |
| AT | SNP_A-8419894 | rs17083201 | 9 | TLE1 | GT | SNP_A-1831539 | rs2144497 | X | DMD | 0.000000 |
| TT | SNP_A-8438227 | rs4686457 | 3 | MASP1 | AA | SNP_A-4218063 | rs10919593 | 1 | PTPRC | 0.000005 |
| TT | SNP_A-8438227 | rs4686457 | 3 | MASP1 | AA | SNP_A-8301485 | rs16902320 | 8 | MYC | 0.000495 |
| TT | SNP_A-8438227 | rs4686457 | 3 | MASP1 | CC | SNP_A-4238494 | rs2862274 | 1 | FAM5B | 0.000332 |
| TT | SNP_A-8438227 | rs4686457 | 3 | MASP1 | CC | SNP_A-8301485 | rs16902320 | 8 | MYC | 0.000316 |
| TT | SNP_A-8438227 | rs4686457 | 3 | MASP1 | TT | SNP_A-2294709 | rs3749796 | 5 | GFPT2 | 0.000391 |
| TT | SNP_A-8438227 | rs4686457 | 3 | MASP1 | TT | SNP_A-2294709 | rs3749796 | 5 | MAPK9 | 0.000391 |
| TT | SNP_A-8438227 | rs4686457 | 3 | MASP1 | TT | SNP_A-4238494 | rs2862274 | 1 | FAM5B | 0.000007 |
| TT | SNP_A-8438227 | rs4686457 | 3 | MASP1 | TT | SNP_A-8554692 | rs11030746 | 11 | METT5D1 | 0.000471 |
| TT | SNP_A-8438227 | rs4686457 | 3 | RTP4 | AA | SNP_A-4218063 | rs10919593 | 1 | PTPRC | 0.000005 |
| TT | SNP_A-8438227 | rs4686457 | 3 | RTP4 | AA | SNP_A-8301485 | rs16902320 | 8 | MYC | 0.000495 |
| TT | SNP_A-8438227 | rs4686457 | 3 | RTP4 | CC | SNP_A-4238494 | rs2862274 | 1 | FAM5B | 0.000332 |
| TT | SNP_A-8438227 | rs4686457 | 3 | RTP4 | CC | SNP_A-8301485 | rs16902320 | 8 | MYC | 0.000316 |
| TT | SNP_A-8438227 | rs4686457 | 3 | RTP4 | TT | SNP_A-2294709 | rs3749796 | 5 | GFPT2 | 0.000391 |
| TT | SNP_A-8438227 | rs4686457 | 3 | RTP4 | TT | SNP_A-2294709 | rs3749796 | 5 | MAPK9 | 0.000391 |
| TT | SNP_A-8438227 | rs4686457 | 3 | RTP4 | TT | SNP_A-4238494 | rs2862274 | 1 | FAM5B | 0.000007 |
| TT | SNP_A-8438227 | rs4686457 | 3 | RTP4 | TT | SNP_A-8554692 | rs11030746 | 11 | METT5D1 | 0.000471 |
| AA | SNP_A-8453929 | rs1991921 | 15 | MEF2A | CC | SNP_A-8554692 | rs11030746 | 11 | METT5D1 | 0.000034 |
| AT | SNP_A-8453929 | rs1991921 | 15 | MEF2A | CC | SNP_A-8554692 | rs11030746 | 11 | METT5D1 | 0.000034 |
| AA | SNP_A-8453929 | rs1991921 | 15 | MEF2A | CC | SNP_A-8716016 | rs3816612 | 15 | ITGA11 | 0.000908 |
| AT | SNP_A-8453929 | rs1991921 | 15 | MEF2A | CC | SNP_A-8716016 | rs3816612 | 15 | ITGA11 | 0.000908 |
| AA | SNP_A-8453929 | rs1991921 | 15 | MEF2A | TT | SNP_A-2294709 | rs3749796 | 5 | GFPT2 | 0.000060 |
| AT | SNP_A-8453929 | rs1991921 | 15 | MEF2A | TT | SNP_A-2294709 | rs3749796 | 5 | GFPT2 | 0.000060 |
| AA | SNP_A-8453929 | rs1991921 | 15 | MEF2A | TT | SNP_A-2294709 | rs3749796 | 5 | MAPK9 | 0.000060 |
| AT | SNP_A-8453929 | rs1991921 | 15 | MEF2A | TT | SNP_A-2294709 | rs3749796 | 5 | MAPK9 | 0.000060 |
| AA | SNP_A-8453929 | rs1991921 | 15 | MEF2A | TT | SNP_A-8554692 | rs11030746 | 11 | METT5D1 | 0.000024 |
| AT | SNP_A-8453929 | rs1991921 | 15 | MEF2A | TT | SNP_A-8554692 | rs11030746 | 11 | METT5D1 | 0.000024 |
| AA | SNP_A-8579220 | rs2762933 | 20 | CYP24A1 | CC | SNP_A-4238494 | rs2862274 | 1 | FAM5B | 0.000727 |
| AT | SNP_A-8579220 | rs2762933 | 20 | CYP24A1 | CC | SNP_A-4238494 | rs2862274 | 1 | FAM5B | 0.000727 |
| AA | SNP_A-8579220 | rs2762933 | 20 | CYP24A1 | TT | SNP_A-4238494 | rs2862274 | 1 | FAM5B | 0.000435 |
| AT | SNP_A-8579220 | rs2762933 | 20 | CYP24A1 | TT | SNP_A-4238494 | rs2862274 | 1 | FAM5B | 0.000435 |
| AA | SNP_A-8579220 | rs2762933 | 20 | BCAS1 | CC | SNP_A-4238494 | rs2862274 | 1 | FAM5B | 0.000727 |
| AT | SNP_A-8579220 | rs2762933 | 20 | BCAS1 | CC | SNP_A-4238494 | rs2862274 | 1 | FAM5B | 0.000727 |
| AA | SNP_A-8579220 | rs2762933 | 20 | BCAS1 | TT | SNP_A-4238494 | rs2862274 | 1 | FAM5B | 0.000435 |
| AT | SNP_A-8579220 | rs2762933 | 20 | BCAS1 | TT | SNP_A-4238494 | rs2862274 | 1 | FAM5B | 0.000435 |
| TT | SNP_A-8619237 | rs1822910 | 3 | GM2AP | AA | SNP_A-8634376 | rs2623149 | 8 | GEM | 0.000502 |
| TT | SNP_A-8619237 | rs1822910 | 3 | GM2AP | AC | SNP_A-8388931 | rs11625236 | 14 | ACTR10 | 0.000857 |
| TT | SNP_A-8619237 | rs1822910 | 3 | GM2AP | AG | SNP_A-8634376 | rs2623149 | 8 | GEM | 0.000502 |
| TT | SNP_A-8619237 | rs1822910 | 3 | GM2AP | CC | SNP_A-8300818 | rs2274703 | 1 | LEMD1 | 0.000810 |
| TT | SNP_A-8619237 | rs1822910 | 3 | GM2AP | CT | SNP_A-8485365 | rs2613216 | 8 | GEM | 0.000502 |
| TT | SNP_A-8619237 | rs1822910 | 3 | GM2AP | TT | SNP_A-8485365 | rs2613216 | 8 | GEM | 0.000502 |
| AA | SNP_A-8663346 | rs17372695 | 4 | DCHS2 | CC | SNP_A-4238494 | rs2862274 | 1 | FAM5B | 0.000829 |
| AT | SNP_A-8663346 | rs17372695 | 4 | DCHS2 | CC | SNP_A-4238494 | rs2862274 | 1 | FAM5B | 0.000829 |
| AA | SNP_A-8663346 | rs17372695 | 4 | DCHS2 | TT | SNP_A-4238494 | rs2862274 | 1 | FAM5B | 0.000751 |
| AT | SNP_A-8663346 | rs17372695 | 4 | DCHS2 | TT | SNP_A-4238494 | rs2862274 | 1 | FAM5B | 0.000751 |
| AA | SNP_A-8663346 | rs17372695 | 4 | SFRP2 | CC | SNP_A-4238494 | rs2862274 | 1 | FAM5B | 0.000829 |
| AT | SNP_A-8663346 | rs17372695 | 4 | SFRP2 | CC | SNP_A-4238494 | rs2862274 | 1 | FAM5B | 0.000829 |
| AA | SNP_A-8663346 | rs17372695 | 4 | SFRP2 | TT | SNP_A-4238494 | rs2862274 | 1 | FAM5B | 0.000751 |
| AT | SNP_A-8663346 | rs17372695 | 4 | SFRP2 | TT | SNP_A-4238494 | rs2862274 | 1 | FAM5B | 0.000751 |
| TT | SNP_A-8673617 | rs2615903 | 11 | HIPK3 | AA | SNP_A-8347387 | rs2525841 | 12 | ACSS3 | 0.000921 |
| TT | SNP_A-8673617 | rs2615903 | 11 | HIPK3 | AG | SNP_A-8446732 | rs17154320 | 7 | SEMA3C | 0.000809 |
| TT | SNP_A-8673617 | rs2615903 | 11 | C11orf41 | AA | SNP_A-2294709 | rs3749796 | 5 | GFPT2 | 0.000966 |
| TT | SNP_A-8673617 | rs2615903 | 11 | C11orf41 | AA | SNP_A-2294709 | rs3749796 | 5 | MAPK9 | 0.000966 |

TABLE 7
Characteristics of Buffering Gene

| Geno. | | | Chr.- | | | | |
|---|---|---|---|---|---|---|---|
| buffering | Buffering | dbSNP-Buffering | Buffering | Location-Buffering | | | Gene-Buffering |
| AA | SNP_A-8446732 | rs17154320 | 7 | 80149730 | downstream | 3201 | CD36 |
| AA | SNP_A-8446732 | rs17154320 | 7 | 80149730 | downstream | 3201 | CD36 |
| AA | SNP_A-8446732 | rs17154320 | 7 | 80149730 | downstream | 3201 | SEMA3C |
| AA | SNP_A-8446732 | rs17154320 | 7 | 80149730 | downstream | 3201 | SEMA3C |
| TT | SNP_A-1865739 | rs6094553 | 20 | 45073866 | intron | 0 | EYA2 |
| AT | SNP_A-1865739 | rs6094553 | 20 | 45073866 | intron | 0 | EYA2 |
| TT | SNP_A-1865739 | rs6094553 | 20 | 45073866 | intron | 0 | EYA2 |
| AT | SNP_A-1865739 | rs6094553 | 20 | 45073866 | intron | 0 | EYA2 |
| TT | SNP_A-2051880 | rs16951040 | 16 | 50892007 | downstream | 138634 | TOX3 |
| AT | SNP_A-2051880 | rs16951040 | 16 | 50892007 | downstream | 138634 | TOX3 |
| AA | SNP_A-2083501 | rs4686870 | 3 | 188500535 | upstream | 8089 | MASP1 |
| AA | SNP_A-2083501 | rs4686870 | 3 | 188500535 | upstream | 8089 | MASP1 |
| AA | SNP_A-2083501 | rs4686870 | 3 | 188500535 | upstream | 8089 | MASP1 |
| AA | SNP_A-2083501 | rs4686870 | 3 | 188500535 | upstream | 8089 | MASP1 |
| AA | SNP_A-2083501 | rs4686870 | 3 | 188500535 | upstream | 8089 | MASP1 |
| AA | SNP_A-2083501 | rs4686870 | 3 | 188500535 | upstream | 8089 | MASP1 |
| AA | SNP_A-2083501 | rs4686870 | 3 | 188500535 | upstream | 8089 | MASP1 |
| AA | SNP_A-2083501 | rs4686870 | 3 | 188500535 | upstream | 8089 | MASP1 |
| AA | SNP_A-2083501 | rs4686870 | 3 | 188500535 | upstream | 68326 | RTP4 |
| AA | SNP_A-2083501 | rs4686870 | 3 | 188500535 | upstream | 68326 | RTP4 |
| AA | SNP_A-2083501 | rs4686870 | 3 | 188500535 | upstream | 68326 | RTP4 |
| AA | SNP_A-2083501 | rs4686870 | 3 | 188500535 | upstream | 68326 | RTP4 |
| AA | SNP_A-2083501 | rs4686870 | 3 | 188500535 | upstream | 68326 | RTP4 |
| AA | SNP_A-2083501 | rs4686870 | 3 | 188500535 | upstream | 68326 | RTP4 |
| AA | SNP_A-2083501 | rs4686870 | 3 | 188500535 | upstream | 68326 | RTP4 |
| AA | SNP_A-2083501 | rs4686870 | 3 | 188500535 | upstream | 68326 | RTP4 |
| TT | SNP_A-2310343 | rs325380 | 15 | 98074141 | 3UTR | 0 | MEF2A |
| AT | SNP_A-2310343 | rs325380 | 15 | 98074141 | 3UTR | 0 | MEF2A |
| TT | SNP_A-2310343 | rs325380 | 15 | 98074141 | 3UTR | 0 | MEF2A |
| AT | SNP_A-2310343 | rs325380 | 15 | 98074141 | 3UTR | 0 | MEF2A |
| TT | SNP_A-2310343 | rs325380 | 15 | 98074141 | 3UTR | 0 | MEF2A |
| AT | SNP_A-2310343 | rs325380 | 15 | 98074141 | 3UTR | 0 | MEF2A |
| TT | SNP_A-2310343 | rs325380 | 15 | 98074141 | 3UTR | 0 | MEF2A |
| AT | SNP_A-2310343 | rs325380 | 15 | 98074141 | 3UTR | 0 | MEF2A |
| TT | SNP_A-4297368 | rs6851158 | 4 | 113998568 | intron | 0 | ANK2 |
| AT | SNP_A-4297368 | rs6851158 | 4 | 113998568 | intron | 0 | ANK2 |
| TT | SNP_A-8328958 | rs16880188 | 5 | 8293188 | downstream | 217451 | LOC729506 |
| AT | SNP_A-8328958 | rs16880188 | 5 | 8293188 | downstream | 217451 | LOC729506 |
| TT | SNP_A-8328958 | rs16880188 | 5 | 8293188 | downstream | 217451 | LOC729506 |
| AT | SNP_A-8328958 | rs16880188 | 5 | 8293188 | downstream | 217451 | LOC729506 |
| TT | SNP_A-8328958 | rs16880188 | 5 | 8293188 | downstream | 217451 | LOC729506 |
| AT | SNP_A-8328958 | rs16880188 | 5 | 8293188 | downstream | 217451 | LOC729506 |
| TT | SNP_A-8328958 | rs16880188 | 5 | 8293188 | downstream | 217451 | LOC729506 |
| AT | SNP_A-8328958 | rs16880188 | 5 | 8293188 | downstream | 217451 | LOC729506 |
| TT | SNP_A-8328958 | rs16880188 | 5 | 8293188 | downstream | 338955 | MTRR |
| AT | SNP_A-8328958 | rs16880188 | 5 | 8293188 | downstream | 338955 | MTRR |
| TT | SNP_A-8328958 | rs16880188 | 5 | 8293188 | downstream | 338955 | MTRR |
| AT | SNP_A-8328958 | rs16880188 | 5 | 8293188 | downstream | 338955 | MTRR |
| TT | SNP_A-8328958 | rs16880188 | 5 | 8293188 | downstream | 338955 | MTRR |
| AT | SNP_A-8328958 | rs16880188 | 5 | 8293188 | downstream | 338955 | MTRR |
| TT | SNP_A-8328958 | rs16880188 | 5 | 8293188 | downstream | 338955 | MTRR |
| AT | SNP_A-8328958 | rs16880188 | 5 | 8293188 | downstream | 338955 | MTRR |
| AA | SNP_A-8347387 | rs2525841 | 12 | 80054239 | intron | 0 | ACSS3 |
| AA | SNP_A-8347387 | rs2525841 | 12 | 80054239 | intron | 0 | ACSS3 |
| TT | SNP_A-8619237 | rs1822910 | 3 | 146474560 | downstream | 411067 | GM2AP |
| AT | SNP_A-8619237 | rs1822910 | 3 | 146474560 | downstream | 411067 | GM2AP |
| TT | SNP_A-8619237 | rs1822910 | 3 | 146474560 | downstream | 411067 | GM2AP |
| AT | SNP_A-8619237 | rs1822910 | 3 | 146474560 | downstream | 411067 | GM2AP |
| AA | SNP_A-8619277 | rs696405 | 3 | 188438917 | intron | 0 | MASP1 |
| TT | SNP_A-8687186 | rs17014445 | 2 | 34058603 | upstream | 252257 | MYADML |
| AT | SNP_A-8687186 | rs17014445 | 2 | 34058603 | upstream | 252257 | MYADML |
| TT | SNP_A-8687186 | rs17014445 | 2 | 34058603 | upstream | 252257 | MYADML |
| AT | SNP_A-8687186 | rs17014445 | 2 | 34058603 | upstream | 252257 | MYADML |
| TT | SNP_A-8687186 | rs17014445 | 2 | 34058603 | upstream | 252257 | MYADML |
| AT | SNP_A-8687186 | rs17014445 | 2 | 34058603 | upstream | 252257 | MYADML |
| TT | SNP_A-8687186 | rs17014445 | 2 | 34058603 | upstream | 252257 | MYADML |
| AT | SNP_A-8687186 | rs17014445 | 2 | 34058603 | upstream | 252257 | MYADML |
| TT | SNP_A-8687186 | rs17014445 | 2 | 34058603 | upstream | 252257 | MYADML |
| AT | SNP_A-8687186 | rs17014445 | 2 | 34058603 | upstream | 252257 | MYADML |
| TT | SNP_A-8687186 | rs17014445 | 2 | 34058603 | upstream | 252257 | MYADML |
| AT | SNP_A-8687186 | rs17014445 | 2 | 34058603 | upstream | 252257 | MYADML |
| TT | SNP_A-8687186 | rs17014445 | 2 | 34058603 | upstream | 252257 | MYADML |
| AT | SNP_A-8687186 | rs17014445 | 2 | 34058603 | upstream | 252257 | MYADML |
| TT | SNP_A-8687186 | rs17014445 | 2 | 34058603 | upstream | 252257 | MYADML |
| AT | SNP_A-8687186 | rs17014445 | 2 | 34058603 | upstream | 252257 | MYADML |
| TT | SNP_A-8687186 | rs17014445 | 2 | 34058603 | downstream | 2376788 | LOC100130506 |
| AT | SNP_A-8687186 | rs17014445 | 2 | 34058603 | downstream | 2376788 | LOC100130506 |
| TT | SNP_A-8687186 | rs17014445 | 2 | 34058603 | downstream | 2376788 | LOC100130506 |
| AT | SNP_A-8687186 | rs17014445 | 2 | 34058603 | downstream | 2376788 | LOC100130506 |
| TT | SNP_A-8687186 | rs17014445 | 2 | 34058603 | upstream | 139290 | SLC25A5 |
| AT | SNP_A-8687186 | rs17014445 | 2 | 34058603 | upstream | 139290 | SLC25A5 |
| TT | SNP_A-8687186 | rs17014445 | 2 | 34058603 | upstream | 139290 | SLC25A5 |
| AT | SNP_A-8687186 | rs17014445 | 2 | 34058603 | upstream | 139290 | SLC25A5 |
| AA | SNP_A-8446732 | rs17154320 | 7 | 80149730 | downstream | 3201 | CD36 |
| GG | SNP_A-8446732 | rs17154320 | 7 | 80149730 | downstream | 3201 | CD36 |
| AA | SNP_A-8446732 | rs17154320 | 7 | 80149730 | downstream | 3201 | CD36 |
| GG | SNP_A-8446732 | rs17154320 | 7 | 80149730 | downstream | 3201 | CD36 |
| AA | SNP_A-8446732 | rs17154320 | 7 | 80149730 | downstream | 3201 | SEMA3C |
| GG | SNP_A-8446732 | rs17154320 | 7 | 80149730 | downstream | 3201 | SEMA3C |
| AA | SNP_A-8446732 | rs17154320 | 7 | 80149730 | downstream | 3201 | SEMA3C |
| GG | SNP_A-8446732 | rs17154320 | 7 | 80149730 | downstream | 3201 | SEMA3C |
| AA | SNP_A-4180725 | rs7106605 | 11 | 103045752 | downstream | 189951 | DYNC2H1 |
| GG | SNP_A-4180725 | rs7106605 | 11 | 103045752 | downstream | 189951 | DYNC2H1 |
| AA | SNP_A-4180725 | rs7106605 | 11 | 103045752 | downstream | 189951 | DYNC2H1 |
| GG | SNP_A-4180725 | rs7106605 | 11 | 103045752 | downstream | 189951 | DYNC2H1 |
| AA | SNP_A-4180725 | rs7106605 | 11 | 103045752 | downstream | 237372 | PDGFD |
| GG | SNP_A-4180725 | rs7106605 | 11 | 103045752 | downstream | 237372 | PDGFD |
| AA | SNP_A-4180725 | rs7106605 | 11 | 103045752 | downstream | 237372 | PDGFD |
| GG | SNP_A-4180725 | rs7106605 | 11 | 103045752 | downstream | 237372 | PDGFD |
| AG | SNP_A-4286901 | rs1528708 | 8 | 35347405 | intron | 0 | UNC5D |
| AG | SNP_A-8282993 | rs2382993 | 8 | 125770106 | intron | 0 | MTSS1 |
| AG | SNP_A-8494528 | rs1255575 | 14 | 62847659 | downstream | 1735 | GPHB5 |
| AG | SNP_A-8494528 | rs1255575 | 14 | 62847659 | downstream | 1735 | GPHB5 |
| AG | SNP_A-8494528 | rs1255575 | 14 | 62847659 | downstream | 19347 | RHOJ |
| AG | SNP_A-8494528 | rs1255575 | 14 | 62847659 | downstream | 19347 | RHOJ |
| AA | SNP_A-8605897 | rs7130910 | 11 | 103050448 | downstream | 194647 | DYNC2H1 |
| GG | SNP_A-8605897 | rs7130910 | 11 | 103050448 | downstream | 194647 | DYNC2H1 |
| AA | SNP_A-8605897 | rs7130910 | 11 | 103050448 | downstream | 194647 | DYNC2H1 |
| GG | SNP_A-8605897 | rs7130910 | 11 | 103050448 | downstream | 194647 | DYNC2H1 |
| AA | SNP_A-8605897 | rs7130910 | 11 | 103050448 | downstream | 232676 | PDGFD |
| GG | SNP_A-8605897 | rs7130910 | 11 | 103050448 | downstream | 232676 | PDGFD |
| AA | SNP_A-8605897 | rs7130910 | 11 | 103050448 | downstream | 232676 | PDGFD |
| GG | SNP_A-8605897 | rs7130910 | 11 | 103050448 | downstream | 232676 | PDGFD |
| CC | SNP_A-1850528 | rs17049044 | 4 | 137958761 | downstream | 700671 | PCDH18 |
| CC | SNP_A-1850528 | rs17049044 | 4 | 137958761 | downstream | 700671 | PCDH18 |
| GG | SNP_A-1905146 | rs2012129 | 15 | 97950440 | intron | 0 | MEF2A |
| CG | SNP_A-1905146 | rs2012129 | 15 | 97950440 | intron | 0 | MEF2A |
| GG | SNP_A-1905146 | rs2012129 | 15 | 97950440 | intron | 0 | MEF2A |
| CG | SNP_A-1905146 | rs2012129 | 15 | 97950440 | intron | 0 | MEF2A |
| GG | SNP_A-1905146 | rs2012129 | 15 | 97950440 | intron | 0 | MEF2A |
| CG | SNP_A-1905146 | rs2012129 | 15 | 97950440 | intron | 0 | MEF2A |
| GG | SNP_A-1905146 | rs2012129 | 15 | 97950440 | intron | 0 | MEF2A |
| CG | SNP_A-1905146 | rs2012129 | 15 | 97950440 | intron | 0 | MEF2A |
| GG | SNP_A-1935646 | rs2098846 | — | — | — | — | |
| CG | SNP_A-1935646 | rs2098846 | — | — | — | — | |
| GG | SNP_A-1935646 | rs2098846 | — | — | — | — | |
| CG | SNP_A-1935646 | rs2098846 | — | — | — | — | |
| CC | SNP_A-2051880 | rs16951040 | 16 | 50892007 | downstream | 138634 | TOX3 |
| GG | SNP_A-4289735 | rs2061007 | 15 | 98087203 | CDS | 0 | LYSMD4 |
| CG | SNP_A-4289735 | rs2061007 | 15 | 98087203 | CDS | 0 | LYSMD4 |
| GG | SNP_A-4289735 | rs2061007 | 15 | 98087203 | CDS | 0 | LYSMD4 |
| CG | SNP_A-4289735 | rs2061007 | 15 | 98087203 | CDS | 0 | LYSMD4 |
| GG | SNP_A-4289735 | rs2061007 | 15 | 98087203 | CDS | 0 | LYSMD4 |
| CG | SNP_A-4289735 | rs2061007 | 15 | 98087203 | CDS | 0 | LYSMD4 |
| GG | SNP_A-4289735 | rs2061007 | 15 | 98087203 | CDS | 0 | LYSMD4 |
| CG | SNP_A-4289735 | rs2061007 | 15 | 98087203 | CDS | 0 | LYSMD4 |
| GG | SNP_A-4289735 | rs2061007 | 15 | 98087203 | CDS | 0 | LYSMD4 |
| CG | SNP_A-4289735 | rs2061007 | 15 | 98087203 | CDS | 0 | LYSMD4 |
| GG | SNP_A-4289735 | rs2061007 | 15 | 98087203 | CDS | 0 | LYSMD4 |
| CG | SNP_A-4289735 | rs2061007 | 15 | 98087203 | CDS | 0 | LYSMD4 |
| GG | SNP_A-4289735 | rs2061007 | 15 | 98087203 | CDS | 0 | LYSMD4 |
| CG | SNP_A-4289735 | rs2061007 | 15 | 98087203 | CDS | 0 | LYSMD4 |
| GG | SNP_A-4289735 | rs2061007 | 15 | 98087203 | CDS | 0 | LYSMD4 |
| CG | SNP_A-4289735 | rs2061007 | 15 | 98087203 | CDS | 0 | LYSMD4 |
| GG | SNP_A-4289735 | rs2061007 | 15 | 98087203 | CDS | 0 | LYSMD4 |
| CG | SNP_A-4289735 | rs2061007 | 15 | 98087203 | CDS | 0 | LYSMD4 |
| GG | SNP_A-4289735 | rs2061007 | 15 | 98087203 | CDS | 0 | LYSMD4 |
| CG | SNP_A-4289735 | rs2061007 | 15 | 98087203 | CDS | 0 | LYSMD4 |
| GG | SNP_A-4289735 | rs2061007 | 15 | 98087203 | CDS | 0 | LYSMD4 |
| CG | SNP_A-4289735 | rs2061007 | 15 | 98087203 | CDS | 0 | LYSMD4 |
| GG | SNP_A-4289735 | rs2061007 | 15 | 98087203 | CDS | 0 | LYSMD4 |
| CG | SNP_A-4289735 | rs2061007 | 15 | 98087203 | CDS | 0 | LYSMD4 |
| CC | SNP_A-8300818 | rs2274703 | 1 | 203620091 | CDS | 0 | LEMD1 |
| CC | SNP_A-8300818 | rs2274703 | 1 | 203620091 | CDS | 0 | LEMD1 |
| CC | SNP_A-8300818 | rs2274703 | 1 | 203620091 | CDS | 0 | LEMD1 |
| GG | SNP_A-8374392 | rs6066159 | 20 | 45074038 | intron | 0 | EYA2 |
| CG | SNP_A-8374392 | rs6066159 | 20 | 45074038 | intron | 0 | EYA2 |
| GG | SNP_A-8374392 | rs6066159 | 20 | 45074038 | intron | 0 | EYA2 |
| CG | SNP_A-8374392 | rs6066159 | 20 | 45074038 | intron | 0 | EYA2 |
| GG | SNP_A-8374392 | rs6066159 | 20 | 45074038 | intron | 0 | EYA2 |
| CG | SNP_A-8374392 | rs6066159 | 20 | 45074038 | intron | 0 | EYA2 |
| GG | SNP_A-8374392 | rs6066159 | 20 | 45074038 | intron | 0 | EYA2 |
| CG | SNP_A-8374392 | rs6066159 | 20 | 45074038 | intron | 0 | EYA2 |
| CC | SNP_A-8452641 | rs1499953 | 3 | 116719791 | upstream | 105049 | GAP43 |
| CC | SNP_A-8452641 | rs1499953 | 3 | 116719791 | upstream | 370974 | ZBTB20 |
| CC | SNP_A-8639378 | rs12432648 | 14 | 39997127 | upstream | 1149396 | LRFN5 |
| CC | SNP_A-8639378 | rs12432648 | 14 | 39997127 | upstream | 1149396 | LRFN5 |
| CG | SNP_A-8451881 | rs10041500 | 5 | 6440741 | upstream | 9102 | FLJ25076 |
| CG | SNP_A-8451881 | rs10041500 | 5 | 6440741 | upstream | 9102 | FLJ25076 |
| CG | SNP_A-8451881 | rs10041500 | 5 | 6440741 | upstream | 9102 | MED10 |
| CG | SNP_A-8451881 | rs10041500 | 5 | 6440741 | upstream | 9102 | MED10 |
| CC | SNP_A-8639378 | rs12432648 | 14 | 39997127 | upstream | 1149396 | LRFN5 |
| GG | SNP_A-8639378 | rs12432648 | 14 | 39997127 | upstream | 1149396 | LRFN5 |
| CC | SNP_A-8639378 | rs12432648 | 14 | 39997127 | upstream | 1149396 | LRFN5 |
| GG | SNP_A-8639378 | rs12432648 | 14 | 39997127 | upstream | 1149396 | LRFN5 |
| CT | SNP_A-2032934 | rs17775764 | 5 | 6458678 | upstream | 27039 | FLJ25076 |
| CT | SNP_A-2032934 | rs17775764 | 5 | 6458678 | upstream | 27039 | FLJ25076 |
| CT | SNP_A-2032934 | rs17775764 | 5 | 6458678 | upstream | 27039 | MED10 |
| CT | SNP_A-2032934 | rs17775764 | 5 | 6458678 | upstream | 27039 | MED10 |
| CT | SNP_A-4291022 | rs5979604 | X | 12274581 | intron | 0 | FRMPD4 |
| CT | SNP_A-4291022 | rs5979604 | X | 12274581 | intron | 0 | FRMPD4 |
| CC | SNP_A-8479146 | rs7153013 | 14 | 79945864 | downstream | 86709 | C14orf145 |
| TT | SNP_A-8479146 | rs7153013 | 14 | 79945864 | downstream | 86709 | C14orf145 |
| CC | SNP_A-8479146 | rs7153013 | 14 | 79945864 | upstream | 197586 | DIO2 |
| TT | SNP_A-8479146 | rs7153013 | 14 | 79945864 | upstream | 197586 | DIO2 |
| CT | SNP_A-8665678 | rs11738547 | 5 | 10087081 | downstream | 192356 | FAM173B |
| CT | SNP_A-8665678 | rs11738547 | 5 | 10087081 | downstream | 192356 | FAM173B |
| CT | SNP_A-8665678 | rs11738547 | 5 | 10087081 | downstream | 192356 | FAM173B |
| CT | SNP_A-8665678 | rs11738547 | 5 | 10087081 | downstream | 192356 | FAM173B |
| CT | SNP_A-8665678 | rs11738547 | 5 | 10087081 | downstream | 192356 | FAM173B |
| CT | SNP_A-8665678 | rs11738547 | 5 | 10087081 | downstream | 192356 | FAM173B |
| CT | SNP_A-8665678 | rs11738547 | 5 | 10087081 | downstream | 192356 | FAM173B |
| CT | SNP_A-8665678 | rs11738547 | 5 | 10087081 | downstream | 192356 | FAM173B |
| CT | SNP_A-8665678 | rs11738547 | 5 | 10087081 | upstream | 403618 | TAS2R1 |
| CT | SNP_A-8665678 | rs11738547 | 5 | 10087081 | upstream | 403618 | TAS2R1 |
| CT | SNP_A-8665678 | rs11738547 | 5 | 10087081 | upstream | 403618 | TAS2R1 |
| CT | SNP_A-8665678 | rs11738547 | 5 | 10087081 | upstream | 403618 | TAS2R1 |
| CT | SNP_A-8665678 | rs11738547 | 5 | 10087081 | upstream | 403618 | TAS2R1 |
| CT | SNP_A-8665678 | rs11738547 | 5 | 10087081 | upstream | 403618 | TAS2R1 |
| CT | SNP_A-8665678 | rs11738547 | 5 | 10087081 | upstream | 403618 | TAS2R1 |
| CT | SNP_A-8665678 | rs11738547 | 5 | 10087081 | upstream | 403618 | TAS2R1 |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| GG | SNP_A-1854119 | rs7221263 | 17 | 39367221 | downstream | 6476 | PPY |
| CC | SNP_A-1996154 | rs10738555 | 9 | 19915073 | downstream | 419894 | MLLT3 |
| GC | SNP_A-1996154 | rs10738555 | 9 | 19915073 | downstream | 419894 | MLLT3 |
| CC | SNP_A-1996154 | rs10738555 | 9 | 19915073 | upstream | 138147 | SLC24A2 |
| GC | SNP_A-1996154 | rs10738555 | 9 | 19915073 | upstream | 138147 | SLC24A2 |
| GG | SNP_A-4272472 | rs1461809 | 3 | 28679611 | intron | 0 | LOC645206 |
| CC | SNP_A-8347387 | rs2525841 | 12 | 80054239 | intron | 0 | ACSS3 |
| CG | SNP_A-8347387 | rs2525841 | 12 | 80054239 | intron | 0 | ACSS3 |
| CC | SNP_A-8347387 | rs2525841 | 12 | 80054239 | intron | 0 | ACSS3 |
| CG | SNP_A-8347387 | rs2525841 | 12 | 80054239 | intron | 0 | ACSS3 |
| CC | SNP_A-8347387 | rs2525841 | 12 | 80054239 | intron | 0 | ACSS3 |
| CG | SNP_A-8347387 | rs2525841 | 12 | 80054239 | intron | 0 | ACSS3 |
| CC | SNP_A-8347387 | rs2525841 | 12 | 80054239 | intron | 0 | ACSS3 |
| CG | SNP_A-8347387 | rs2525841 | 12 | 80054239 | intron | 0 | ACSS3 |
| CC | SNP_A-8384021 | rs368866 | X | 88703924 | downstream | 807482 | CPXCR1 |
| CG | SNP_A-8384021 | rs368866 | X | 88703924 | downstream | 807482 | CPXCR1 |
| CC | SNP_A-8384021 | rs368866 | X | 88703924 | downstream | 807482 | CPXCR1 |
| CG | SNP_A-8384021 | rs368866 | X | 88703924 | downstream | 807482 | CPXCR1 |
| CC | SNP_A-8384021 | rs368866 | X | 88703924 | upstream | 359656 | TGIF2LY |
| CG | SNP_A-8384021 | rs368866 | X | 88703924 | upstream | 359656 | TGIF2LY |
| CC | SNP_A-8384021 | rs368866 | X | 88703924 | upstream | 359656 | TGIF2LY |
| CG | SNP_A-8384021 | rs368866 | X | 88703924 | upstream | 359656 | TGIF2LY |
| CC | SNP_A-8484091 | rs12348066 | 9 | 9900028 | intron | 0 | PTPRD |
| CG | SNP_A-8484091 | rs12348066 | 9 | 9900028 | intron | 0 | PTPRD |
| CC | SNP_A-8484091 | rs12348066 | 9 | 9900028 | intron | 0 | PTPRD |
| CG | SNP_A-8484091 | rs12348066 | 9 | 9900028 | intron | 0 | PTPRD |
| CC | SNP_A-8484091 | rs12348066 | 9 | 9900028 | intron | 0 | PTPRD |
| CG | SNP_A-8484091 | rs12348066 | 9 | 9900028 | intron | 0 | PTPRD |
| CC | SNP_A-8484091 | rs12348066 | 9 | 9900028 | intron | 0 | PTPRD |
| CG | SNP_A-8484091 | rs12348066 | 9 | 9900028 | intron | 0 | PTPRD |
| CC | SNP_A-8484091 | rs12348066 | 9 | 9900028 | intron | 0 | PTPRD |
| CG | SNP_A-8484091 | rs12348066 | 9 | 9900028 | intron | 0 | PTPRD |
| CC | SNP_A-8484091 | rs12348066 | 9 | 9900028 | intron | 0 | PTPRD |
| CG | SNP_A-8484091 | rs12348066 | 9 | 9900028 | intron | 0 | PTPRD |
| CC | SNP_A-8484091 | rs12348066 | 9 | 9900028 | intron | 0 | PTPRD |
| CG | SNP_A-8484091 | rs12348066 | 9 | 9900028 | intron | 0 | PTPRD |
| CC | SNP_A-8484091 | rs12348066 | 9 | 9900028 | intron | 0 | PTPRD |
| CG | SNP_A-8484091 | rs12348066 | 9 | 9900028 | intron | 0 | PTPRD |
| CC | SNP_A-8494528 | rs1255575 | 14 | 62847659 | downstream | 1735 | GPHB5 |
| CG | SNP_A-8494528 | rs1255575 | 14 | 62847659 | downstream | 1735 | GPHB5 |
| CC | SNP_A-8494528 | rs1255575 | 14 | 62847659 | downstream | 1735 | GPHB5 |
| CG | SNP_A-8494528 | rs1255575 | 14 | 62847659 | downstream | 1735 | GPHB5 |
| CC | SNP_A-8494528 | rs1255575 | 14 | 62847659 | downstream | 19347 | RHOJ |
| CG | SNP_A-8494528 | rs1255575 | 14 | 62847659 | downstream | 19347 | RHOJ |
| CC | SNP_A-8494528 | rs1255575 | 14 | 62847659 | downstream | 19347 | RHOJ |
| CG | SNP_A-8494528 | rs1255575 | 14 | 62847659 | downstream | 19347 | RHOJ |
| GG | SNP_A-8574340 | rs7579939 | 2 | 51842138 | downstream | 1908483 | ASB3 |
| GG | SNP_A-8574340 | rs7579939 | 2 | 51842138 | downstream | 1908483 | ASB3 |
| GG | SNP_A-8574340 | rs7579939 | 2 | 51842138 | upstream | 728960 | NRXN1 |
| GG | SNP_A-8574340 | rs7579939 | 2 | 51842138 | upstream | 728960 | NRXN1 |
| GG | SNP_A-8580357 | rs1430707 | 2 | 131282490 | downstream | 40313 | FAM123C |
| GG | SNP_A-8580357 | rs1430707 | 2 | 131282490 | downstream | 40313 | FAM123C |
| GG | SNP_A-8580357 | rs1430707 | 2 | 131282490 | downstream | 40313 | FAM123C |
| GG | SNP_A-8580357 | rs1430707 | 2 | 131282490 | downstream | 40313 | FAM123C |
| GG | SNP_A-8580357 | rs1430707 | 2 | 131282490 | downstream | 40313 | FAM123C |
| GG | SNP_A-8580357 | rs1430707 | 2 | 131282490 | upstream | 27899 | LOC730032 |
| GG | SNP_A-8580357 | rs1430707 | 2 | 131282490 | upstream | 27899 | LOC730032 |
| GG | SNP_A-8580357 | rs1430707 | 2 | 131282490 | upstream | 27899 | LOC730032 |
| GG | SNP_A-8580357 | rs1430707 | 2 | 131282490 | upstream | 27899 | LOC730032 |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | upstream | 252257 | MYADML |
| GG | SNP_A-8687186 | rs17014445 | 2 | 34058603 | downstream | 2376788 | LOC100130506 |
| GG | SNP_A-8687186 | rs17014445 | 2 | 34058603 | downstream | 2376788 | LOC100130506 |
| GG | SNP_A-8687186 | rs17014445 | 2 | 34058603 | downstream | 2376788 | LOC100130506 |
| GG | SNP_A-8687186 | rs17014445 | 2 | 34058603 | downstream | 2376788 | LOC100130506 |
| GG | SNP_A-8687186 | rs17014445 | 2 | 34058603 | downstream | 2376788 | LOC100130506 |
| GG | SNP_A-8687186 | rs17014445 | 2 | 34058603 | upstream | 139290 | SLC25A5 |
| GG | SNP_A-8687186 | rs17014445 | 2 | 34058603 | upstream | 139290 | SLC25A5 |
| GG | SNP_A-8687186 | rs17014445 | 2 | 34058603 | upstream | 139290 | SLC25A5 |
| GG | SNP_A-8687186 | rs17014445 | 2 | 34058603 | upstream | 139290 | SLC25A5 |
| GG | SNP_A-8687186 | rs17014445 | 2 | 34058603 | upstream | 139290 | SLC25A5 |
| GG | SNP_A-8687186 | rs17014445 | 2 | 34058603 | upstream | 139290 | SLC25A5 |
| GG | SNP_A-8687186 | rs17014445 | 2 | 34058603 | upstream | 139290 | SLC25A5 |
| TT | SNP_A-8438227 | rs4686457 | 3 | 188502086 | upstream | 9640 | MASP1 |
| TT | SNP_A-8438227 | rs4686457 | 3 | 188502086 | upstream | 9640 | MASP1 |
| TT | SNP_A-8438227 | rs4686457 | 3 | 188502086 | upstream | 9640 | MASP1 |
| TT | SNP_A-8438227 | rs4686457 | 3 | 188502086 | upstream | 9640 | RTP4 |
| TT | SNP_A-8438227 | rs4686457 | 3 | 188502086 | upstream | 9640 | RTP4 |
| TT | SNP_A-8438227 | rs4686457 | 3 | 188502086 | upstream | 9640 | RTP4 |
| TT | SNP_A-8673617 | rs2615903 | 11 | 33414403 | downstream | 81976 | HIPK3 |
| TT | SNP_A-8673617 | rs2615903 | 11 | 33414403 | downstream | 81976 | HIPK3 |
| AA | SNP_A-1850528 | rs17049044 | 4 | 137958761 | downstream | 700671 | PCDH18 |
| AT | SNP_A-1850528 | rs17049044 | 4 | 137958761 | downstream | 700671 | PCDH18 |
| TT | SNP_A-2298975 | rs12276840 | 11 | 131853608 | intron | 0 | OPCML |
| TT | SNP_A-2298975 | rs12276840 | 11 | 131853608 | intron | 0 | OPCML |
| AA | SNP_A-8369202 | rs533391 | 13 | 96091455 | intron | 0 | HS6ST3 |
| AT | SNP_A-8369202 | rs533391 | 13 | 96091455 | intron | 0 | HS6ST3 |
| AA | SNP_A-8419894 | rs17083201 | 9 | 82164366 | downstream | 1224053 | TLE1 |
| AT | SNP_A-8419894 | rs17083201 | 9 | 82164366 | downstream | 1224053 | TLE1 |
| TT | SNP_A-8438227 | rs4686457 | 3 | 188502086 | upstream | 9640 | MASP1 |
| TT | SNP_A-8438227 | rs4686457 | 3 | 188502086 | upstream | 9640 | MASP1 |
| TT | SNP_A-8438227 | rs4686457 | 3 | 188502086 | upstream | 9640 | MASP1 |
| TT | SNP_A-8438227 | rs4686457 | 3 | 188502086 | upstream | 9640 | MASP1 |
| TT | SNP_A-8438227 | rs4686457 | 3 | 188502086 | upstream | 9640 | MASP1 |
| TT | SNP_A-8438227 | rs4686457 | 3 | 188502086 | upstream | 9640 | MASP1 |
| TT | SNP_A-8438227 | rs4686457 | 3 | 188502086 | upstream | 9640 | MASP1 |
| TT | SNP_A-8438227 | rs4686457 | 3 | 188502086 | upstream | 9640 | MASP1 |
| TT | SNP_A-8438227 | rs4686457 | 3 | 188502086 | upstream | 66775 | RTP4 |
| TT | SNP_A-8438227 | rs4686457 | 3 | 188502086 | upstream | 66775 | RTP4 |
| TT | SNP_A-8438227 | rs4686457 | 3 | 188502086 | upstream | 66775 | RTP4 |
| TT | SNP_A-8438227 | rs4686457 | 3 | 188502086 | upstream | 66775 | RTP4 |
| TT | SNP_A-8438227 | rs4686457 | 3 | 188502086 | upstream | 66775 | RTP4 |
| TT | SNP_A-8438227 | rs4686457 | 3 | 188502086 | upstream | 66775 | RTP4 |
| TT | SNP_A-8438227 | rs4686457 | 3 | 188502086 | upstream | 66775 | RTP4 |
| TT | SNP_A-8438227 | rs4686457 | 3 | 188502086 | upstream | 66775 | RTP4 |
| AA | SNP_A-8453929 | rs1991921 | 15 | 98011512 | intron | 0 | MEF2A |
| AT | SNP_A-8453929 | rs1991921 | 15 | 98011512 | intron | 0 | MEF2A |
| AA | SNP_A-8453929 | rs1991921 | 15 | 98011512 | intron | 0 | MEF2A |
| AT | SNP_A-8453929 | rs1991921 | 15 | 98011512 | intron | 0 | MEF2A |
| AA | SNP_A-8453929 | rs1991921 | 15 | 98011512 | intron | 0 | MEF2A |
| AT | SNP_A-8453929 | rs1991921 | 15 | 98011512 | intron | 0 | MEF2A |
| AA | SNP_A-8453929 | rs1991921 | 15 | 98011512 | intron | 0 | MEF2A |
| AT | SNP_A-8453929 | rs1991921 | 15 | 98011512 | intron | 0 | MEF2A |
| AA | SNP_A-8453929 | rs1991921 | 15 | 98011512 | intron | 0 | MEF2A |
| AT | SNP_A-8453929 | rs1991921 | 15 | 98011512 | intron | 0 | MEF2A |
| AA | SNP_A-8579220 | rs2762933 | 20 | 52203310 | downstream | 84 | CYP24A1 |
| AT | SNP_A-8579220 | rs2762933 | 20 | 52203310 | downstream | 84 | CYP24A1 |

TABLE 8
Characteristics of Buffered Gene

| Geno. | | | Chr.- | | | | | |
|---|---|---|---|---|---|---|---|---|
| buffered | Buffered | dbSNP-Buffered | Buffered | Location-Buffered | | | Gene-Buffering | P-value |
| CC | SNP_A-8465134 | rs10278652 | 7 | 155338093 | upstream | 40365 | SHH | 0.000005 |
| CC | SNP_A-8465134 | rs10278652 | 7 | 155338093 | upstream | 251780 | tcag7.1213 | 0.000005 |
| CC | SNP_A-8465134 | rs10278652 | 7 | 155338093 | upstream | 40365 | SHH | 0.000005 |
| CC | SNP_A-8465134 | rs10278652 | 7 | 155338093 | upstream | 251780 | tcag7.1213 | 0.000005 |
| CC | SNP_A-8465134 | rs10278652 | 7 | 155338093 | upstream | 40365 | SHH | 0.000001 |
| CC | SNP_A-8465134 | rs10278652 | 7 | 155338093 | upstream | 40365 | SHH | 0.000001 |
| CC | SNP_A-8465134 | rs10278652 | 7 | 155338093 | upstream | 251780 | tcag7.1213 | 0.000001 |
| CC | SNP_A-8465134 | rs10278652 | 7 | 155338093 | upstream | 251780 | tcag7.1213 | 0.000001 |
| CC | SNP_A-4296086 | rs1324362 | 1 | 64339447 | intron | 0 | ROR1 | 0.000410 |
| CC | SNP_A-4296086 | rs1324362 | 1 | 64339447 | intron | 0 | ROR1 | 0.000410 |
| AA | SNP_A-2294709 | rs3749796 | 5 | 179653982 | downstream | 6314 | GFPT2 | 0.000024 |
| AA | SNP_A-2294709 | rs3749796 | 5 | 179653982 | upstream | 13764 | MAPK9 | 0.000024 |
| AA | SNP_A-4218063 | rs10919593 | 1 | 197077995 | downstream | 16630 | PTPRC | 0.000017 |
| CC | SNP_A-4238494 | rs2862274 | 1 | 175945277 | downstream | 427096 | FAM5B | 0.000694 |
| CC | SNP_A-8301485 | rs16902320 | 8 | 128779894 | upstream | 37604 | MYC | 0.000820 |
| TT | SNP_A-2294709 | rs3749796 | 5 | 179653982 | downstream | 6314 | GFPT2 | 0.000815 |
| TT | SNP_A-2294709 | rs3749796 | 5 | 179653982 | upstream | 13764 | MAPK9 | 0.000815 |
| TT | SNP_A-4238494 | rs2862274 | 1 | 175945277 | downstream | 427096 | FAM5B | 0.000020 |
| AA | SNP_A-2294709 | rs3749796 | 5 | 179653982 | downstream | 6314 | GFPT2 | 0.000024 |
| AA | SNP_A-2294709 | rs3749796 | 5 | 179653982 | upstream | 13764 | MAPK9 | 0.000024 |
| AA | SNP_A-4218063 | rs10919593 | 1 | 197077995 | downstream | 16630 | PTPRC | 0.000017 |
| CC | SNP_A-4238494 | rs2862274 | 1 | 175945277 | downstream | 427096 | FAM5B | 0.000694 |
| CC | SNP_A-8301485 | rs16902320 | 8 | 128779894 | upstream | 37604 | MYC | 0.000820 |
| TT | SNP_A-2294709 | rs3749796 | 5 | 179653982 | downstream | 6314 | GFPT2 | 0.000815 |
| TT | SNP_A-2294709 | rs3749796 | 5 | 179653982 | upstream | 13764 | MAPK9 | 0.000815 |
| TT | SNP_A-4238494 | rs2862274 | 1 | 175945277 | downstream | 427096 | FAM5B | 0.000020 |
| CC | SNP_A-8554692 | rs11030746 | 11 | 29592649 | upstream | 106940 | METT5D1 | 0.000179 |
| CC | SNP_A-8554692 | rs11030746 | 11 | 29592649 | upstream | 106940 | METT5D1 | 0.000179 |
| TT | SNP_A-2294709 | rs3749796 | 5 | 179653982 | downstream | 6314 | GFPT2 | 0.000097 |
| TT | SNP_A-2294709 | rs3749796 | 5 | 179653982 | downstream | 6314 | GFPT2 | 0.000097 |
| TT | SNP_A-2294709 | rs3749796 | 5 | 179653982 | upstream | 13764 | MAPK9 | 0.000097 |
| TT | SNP_A-2294709 | rs3749796 | 5 | 179653982 | upstream | 13764 | MAPK9 | 0.000097 |
| TT | SNP_A-8554692 | rs11030746 | 11 | 29592649 | upstream | 106940 | METT5D1 | 0.000144 |
| TT | SNP_A-8554692 | rs11030746 | 11 | 29592649 | upstream | 106940 | METT5D1 | 0.000144 |
| GG | SNP_A-4282690 | rs17120428 | 1 | 60494495 | upstream | 609024 | NFIA | 0.000676 |
| GG | SNP_A-4282690 | rs17120428 | 1 | 60494495 | upstream | 609024 | NFIA | 0.000676 |
| CC | SNP_A-4262900 | rs17003277 | 21 | 14782024 | intron | 0 | SAMSN1 | 0.000181 |
| CC | SNP_A-4262900 | rs17003277 | 21 | 14782024 | intron | 0 | SAMSN1 | 0.000181 |
| CC | SNP_A-8554692 | rs11030746 | 11 | 29592649 | upstream | 106940 | METT5D1 | 0.000173 |
| CC | SNP_A-8554692 | rs11030746 | 11 | 29592649 | upstream | 106940 | METT5D1 | 0.000173 |
| TT | SNP_A-4262900 | rs17003277 | 21 | 14782024 | intron | 0 | SAMSN1 | 0.000138 |
| TT | SNP_A-4262900 | rs17003277 | 21 | 14782024 | intron | 0 | SAMSN1 | 0.000138 |
| TT | SNP_A-8554692 | rs11030746 | 11 | 29592649 | upstream | 106940 | METT5D1 | 0.000188 |
| TT | SNP_A-8554692 | rs11030746 | 11 | 29592649 | upstream | 106940 | METT5D1 | 0.000188 |
| CC | SNP_A-4262900 | rs17003277 | 21 | 14782024 | intron | 0 | SAMSN1 | 0.000181 |
| CC | SNP_A-4262900 | rs17003277 | 21 | 14782024 | intron | 0 | SAMSN1 | 0.000181 |
| CC | SNP_A-8554692 | rs11030746 | 11 | 29592649 | upstream | 106940 | METT5D1 | 0.000173 |
| CC | SNP_A-8554692 | rs11030746 | 11 | 29592649 | upstream | 106940 | METT5D1 | 0.000173 |
| TT | SNP_A-4262900 | rs17003277 | 21 | 14782024 | intron | 0 | SAMSN1 | 0.000138 |
| TT | SNP_A-4262900 | rs17003277 | 21 | 14782024 | intron | 0 | SAMSN1 | 0.000138 |
| TT | SNP_A-8554692 | rs11030746 | 11 | 29592649 | upstream | 106940 | METT5D1 | 0.000188 |
| TT | SNP_A-8554692 | rs11030746 | 11 | 29592649 | upstream | 106940 | METT5D1 | 0.000188 |
| CT | SNP_A-8682320 | rs12838423 | X | 4482942 | downstream | 1335142 | NLGN4X | 0.000190 |
| GT | SNP_A-8456832 | rs12857956 | X | 4452855 | downstream | 1365229 | NLGN4X | 0.000190 |
| GG | SNP_A-2267424 | rs5981167 | X | 68251184 | downstream | 46238 | PJA1 | 0.000805 |
| GG | SNP_A-2267424 | rs5981167 | X | 68251184 | downstream | 46238 | PJA1 | 0.000805 |
| GG | SNP_A-2267424 | rs5981167 | X | 68251184 | downstream | 272455 | EFNB1 | 0.000805 |
| GG | SNP_A-2267424 | rs5981167 | X | 68251184 | downstream | 272455 | EFNB1 | 0.000805 |
| TT | SNP_A-4238494 | rs2862274 | 1 | 175945277 | downstream | 427096 | FAM5B | 0.000417 |
| CC | SNP_A-1797721 | rs11183915 | 12 | 46057251 | upstream | 515 | LOC728148 | 0.000456 |
| CC | SNP_A-1797721 | rs11183915 | 12 | 46057251 | upstream | 515 | LOC728148 | 0.000456 |
| CC | SNP_A-4296112 | rs11103184 | 9 | 137811533 | intron | 0 | KCNT1 | 0.000029 |
| CC | SNP_A-4296112 | rs11103184 | 9 | 137811533 | intron | 0 | KCNT1 | 0.000029 |
| CC | SNP_A-8691130 | rs11103512 | 9 | 136796458 | intron | 0 | COL5A1 | 0.000078 |
| CC | SNP_A-8691130 | rs11103512 | 9 | 136796458 | intron | 0 | COL5A1 | 0.000078 |
| CT | SNP_A-8449633 | rs9586459 | 13 | 103750145 | upstream | 1166219 | DAOA | 0.000045 |
| CT | SNP_A-8449633 | rs9586459 | 13 | 103750145 | upstream | 1166219 | DAOA | 0.000045 |
| GG | SNP_A-4218063 | rs10919593 | 1 | 197077995 | downstream | 84827 | PTPRC | 0.000807 |
| GG | SNP_A-4218063 | rs10919593 | 1 | 197077995 | downstream | 84827 | PTPRC | 0.000807 |
| GG | SNP_A-4218063 | rs10919593 | 1 | 197077995 | upstream | 106760 | RPS2P9 | 0.000807 |
| GG | SNP_A-4218063 | rs10919593 | 1 | 197077995 | upstream | 106760 | RPS2P9 | 0.000807 |
| TT | SNP_A-8449633 | rs9586459 | 13 | 103750145 | upstream | 1166219 | DAOA | 0.000045 |
| TT | SNP_A-8449633 | rs9586459 | 13 | 103750145 | upstream | 1166219 | DAOA | 0.000045 |
| TT | SNP_A-8619237 | rs1822910 | 3 | 146474560 | downstream | 547087 | RPL21P46 | 0.000745 |
| TT | SNP_A-8619237 | rs1822910 | 3 | 146474560 | downstream | 547087 | RPL21P46 | 0.000745 |
| CC | SNP_A-4296112 | rs11103184 | 9 | 137811533 | intron | 0 | KCNT1 | 0.000029 |
| CC | SNP_A-4296112 | rs11103184 | 9 | 137811533 | intron | 0 | KCNT1 | 0.000029 |
| GG | SNP_A-4218063 | rs10919593 | 1 | 197077995 | downstream | 16630 | PTPRC | 0.000807 |
| GG | SNP_A-4218063 | rs10919593 | 1 | 197077995 | downstream | 16630 | PTPRC | 0.000807 |
| CC | SNP_A-4296112 | rs11103184 | 9 | 137811533 | intron | 0 | KCNT1 | 0.000029 |
| CC | SNP_A-4296112 | rs11103184 | 9 | 137811533 | intron | 0 | KCNT1 | 0.000029 |
| GG | SNP_A-4218063 | rs10919593 | 1 | 197077995 | downstream | 16630 | PTPRC | 0.000807 |
| GG | SNP_A-4218063 | rs10919593 | 1 | 197077995 | downstream | 16630 | PTPRC | 0.000807 |
| CC | SNP_A-8465134 | rs10278652 | 7 | 155338093 | upstream | 40365 | SHH | 0.000004 |
| CC | SNP_A-8465134 | rs10278652 | 7 | 155338093 | upstream | 40365 | SHH | 0.000004 |
| CC | SNP_A-8465134 | rs10278652 | 7 | 155338093 | upstream | 251780 | tcag7.1213 | 0.000004 |
| CC | SNP_A-8465134 | rs10278652 | 7 | 155338093 | upstream | 251780 | tcag7.1213 | 0.000004 |
| CC | SNP_A-8465134 | rs10278652 | 7 | 155338093 | upstream | 40365 | SHH | 0.000004 |
| CC | SNP_A-8465134 | rs10278652 | 7 | 155338093 | upstream | 40365 | SHH | 0.000004 |
| CC | SNP_A-8465134 | rs10278652 | 7 | 155338093 | upstream | 251780 | tcag7.1213 | 0.000004 |
| CC | SNP_A-8465134 | rs10278652 | 7 | 155338093 | upstream | 251780 | tcag7.1213 | 0.000004 |
| AA | SNP_A-2294709 | rs3749796 | 5 | 179653982 | downstream | 6314 | GFPT2 | 0.000260 |
| AA | SNP_A-2294709 | rs3749796 | 5 | 179653982 | downstream | 6314 | GFPT2 | 0.000260 |
| AA | SNP_A-2294709 | rs3749796 | 5 | 179653982 | upstream | 13764 | MAPK9 | 0.000260 |
| AA | SNP_A-2294709 | rs3749796 | 5 | 179653982 | upstream | 13764 | MAPK9 | 0.000260 |
| AA | SNP_A-2294709 | rs3749796 | 5 | 179653982 | downstream | 6314 | GFPT2 | 0.000260 |
| AA | SNP_A-2294709 | rs3749796 | 5 | 179653982 | downstream | 6314 | GFPT2 | 0.000260 |
| AA | SNP_A-2294709 | rs3749796 | 5 | 179653982 | upstream | 13764 | MAPK9 | 0.000260 |
| AA | SNP_A-2294709 | rs3749796 | 5 | 179653982 | upstream | 13764 | MAPK9 | 0.000260 |
| GG | SNP_A-8478767 | rs4595891 | 18 | 62571504 | upstream | 149308 | CDH19 | 0.000671 |
| CC | SNP_A-4296112 | rs11103184 | 9 | 137811533 | intron | 0 | KCNT1 | 0.000452 |
| CC | SNP_A-4238494 | rs2862274 | 1 | 175945277 | downstream | 427096 | FAM5B | 0.000932 |
| TT | SNP_A-4238494 | rs2862274 | 1 | 175945277 | downstream | 427096 | FAM5B | 0.000015 |
| CC | SNP_A-4238494 | rs2862274 | 1 | 175945277 | downstream | 427096 | FAM5B | 0.000932 |
| TT | SNP_A-4238494 | rs2862274 | 1 | 175945277 | downstream | 427096 | FAM5B | 0.000015 |
| AA | SNP_A-2294709 | rs3749796 | 5 | 179653982 | downstream | 6314 | GFPT2 | 0.000332 |
| AA | SNP_A-2294709 | rs3749796 | 5 | 179653982 | downstream | 6314 | GFPT2 | 0.000332 |
| AA | SNP_A-2294709 | rs3749796 | 5 | 179653982 | upstream | 13764 | MAPK9 | 0.000332 |
| AA | SNP_A-2294709 | rs3749796 | 5 | 179653982 | upstream | 13764 | MAPK9 | 0.000332 |
| AA | SNP_A-2294709 | rs3749796 | 5 | 179653982 | downstream | 6314 | GFPT2 | 0.000332 |
| AA | SNP_A-2294709 | rs3749796 | 5 | 179653982 | downstream | 6314 | GFPT2 | 0.000332 |
| AA | SNP_A-2294709 | rs3749796 | 5 | 179653982 | upstream | 13764 | MAPK9 | 0.000332 |
| AA | SNP_A-2294709 | rs3749796 | 5 | 179653982 | upstream | 13764 | MAPK9 | 0.000332 |
| AA | SNP_A-8529547 | rs553369 | X | 154616633 | upstream | 34011 | SPRY3 | 0.000930 |
| AA | SNP_A-8529547 | rs553369 | X | 154616633 | upstream | 120822 | TMLHE | 0.000930 |
| CC | SNP_A-8554692 | rs11030746 | 11 | 29592649 | upstream | 106940 | METT5D1 | 0.000102 |
| CC | SNP_A-8554692 | rs11030746 | 11 | 29592649 | upstream | 106940 | METT5D1 | 0.000102 |
| TT | SNP_A-2294709 | rs3749796 | 5 | 179653982 | downstream | 6314 | GFPT2 | 0.000123 |
| TT | SNP_A-2294709 | rs3749796 | 5 | 179653982 | downstream | 6314 | GFPT2 | 0.000123 |
| TT | SNP_A-2294709 | rs3749796 | 5 | 179653982 | upstream | 13764 | MAPK9 | 0.000123 |
| TT | SNP_A-2294709 | rs3749796 | 5 | 179653982 | upstream | 13764 | MAPK9 | 0.000123 |
| TT | SNP_A-8554692 | rs11030746 | 11 | 29592649 | upstream | 106940 | METT5D1 | 0.000070 |
| TT | SNP_A-8554692 | rs11030746 | 11 | 29592649 | upstream | 106940 | METT5D1 | 0.000070 |
| CC | SNP_A-4262900 | rs17003277 | 21 | 14782024 | intron | 0 | SAMSN1 | 0.000586 |
| CC | SNP_A-4262900 | rs17003277 | 21 | 14782024 | intron | 0 | SAMSN1 | 0.000586 |
| TT | SNP_A-4262900 | rs17003277 | 21 | 14782024 | intron | 0 | SAMSN1 | 0.000819 |
| TT | SNP_A-4262900 | rs17003277 | 21 | 14782024 | intron | 0 | SAMSN1 | 0.000819 |
| AG | SNP_A-8446732 | rs17154320 | 7 | 80149730 | downstream | 60060 | SEMA3C | 0.000670 |
| AA | SNP_A-8301485 | rs16902320 | 8 | 128779894 | upstream | 37604 | MYC | 0.000364 |
| AA | SNP_A-8301485 | rs16902320 | 8 | 128779894 | upstream | 37604 | MYC | 0.000364 |
| CC | SNP_A-4238494 | rs2862274 | 1 | 175945277 | downstream | 427096 | FAM5B | 0.000265 |
| CC | SNP_A-4238494 | rs2862274 | 1 | 175945277 | downstream | 427096 | FAM5B | 0.000265 |
| CC | SNP_A-4262900 | rs17003277 | 21 | 14782024 | intron | 0 | SAMSN1 | 0.000060 |
| CC | SNP_A-4262900 | rs17003277 | 21 | 14782024 | intron | 0 | SAMSN1 | 0.000060 |
| CC | SNP_A-8301485 | rs16902320 | 8 | 128779894 | upstream | 37604 | MYC | 0.000213 |
| CC | SNP_A-8301485 | rs16902320 | 8 | 128779894 | upstream | 37604 | MYC | 0.000213 |
| CC | SNP_A-8554692 | rs11030746 | 11 | 29592649 | upstream | 106940 | METT5D1 | 0.000179 |
| CC | SNP_A-8554692 | rs11030746 | 11 | 29592649 | upstream | 106940 | METT5D1 | 0.000179 |
| CC | SNP_A-8716016 | rs3816612 | 15 | 66438234 | intron | 0 | ITGA11 | 0.000047 |
| CC | SNP_A-8716016 | rs3816612 | 15 | 66438234 | intron | 0 | ITGA11 | 0.000047 |
| TT | SNP_A-2294709 | rs3749796 | 5 | 179653982 | downstream | 6314 | GFPT2 | 0.000099 |
| TT | SNP_A-2294709 | rs3749796 | 5 | 179653982 | downstream | 6314 | GFPT2 | 0.000099 |
| TT | SNP_A-2294709 | rs3749796 | 5 | 179653982 | upstream | 13764 | MAPK9 | 0.000099 |
| TT | SNP_A-2294709 | rs3749796 | 5 | 179653982 | upstream | 13764 | MAPK9 | 0.000099 |
| TT | SNP_A-4238494 | rs2862274 | 1 | 175945277 | downstream | 427096 | FAM5B | 0.000247 |
| TT | SNP_A-4238494 | rs2862274 | 1 | 175945277 | downstream | 427096 | FAM5B | 0.000247 |
| TT | SNP_A-8554692 | rs11030746 | 11 | 29592649 | upstream | 106940 | METT5D1 | 0.000145 |
| TT | SNP_A-8554692 | rs11030746 | 11 | 29592649 | upstream | 106940 | METT5D1 | 0.000145 |
| TT | SNP_A-8619237 | rs1822910 | 3 | 146474560 | downstream | 547087 | RPL21P46 | 0.000000 |
| TT | SNP_A-8619237 | rs1822910 | 3 | 146474560 | downstream | 547087 | RPL21P46 | 0.000000 |
| TT | SNP_A-8716016 | rs3816612 | 15 | 66438234 | intron | 0 | ITGA11 | 0.000381 |
| TT | SNP_A-8716016 | rs3816612 | 15 | 66438234 | intron | 0 | ITGA11 | 0.000381 |
| CC | SNP_A-8475711 | rs7827228 | 8 | 2577400 | downstream | 202881 | CSMD1 | 0.000490 |
| CC | SNP_A-8475711 | rs7827228 | 8 | 2577400 | downstream | 496614 | MYOM2 | 0.000490 |
| CG | SNP_A-1881982 | rs2237342 | 7 | 27762767 | intron | — | TAX1BP1 | 0.000234 |
| AA | SNP_A-4218063 | rs10919593 | 1 | 197077995 | downstream | 16630 | PTPRC | 0.000576 |
| AA | SNP_A-4218063 | rs10919593 | 1 | 197077995 | downstream | 16630 | PTPRC | 0.000576 |
| AA | SNP_A-8636331 | rs10920481 | 1 | 201024173 | intron | 0 | KDM5B | 0.000007 |
| AA | SNP_A-8636331 | rs10920481 | 1 | 201024173 | intron | 0 | KDM5B | 0.000007 |
| CC | SNP_A-8465134 | rs10278652 | 7 | 155338093 | upstream | 40365 | SHH | 0.000001 |
| CC | SNP_A-8465134 | rs10278652 | 7 | 155338093 | upstream | 40365 | SHH | 0.000001 |
| CC | SNP_A-8465134 | rs10278652 | 7 | 155338093 | upstream | 251780 | tcag7.1213 | 0.000001 |
| CC | SNP_A-8465134 | rs10278652 | 7 | 155338093 | upstream | 251780 | tcag7.1213 | 0.000001 |
| AA | SNP_A-4218063 | rs10919593 | 1 | 197077995 | downstream | 16630 | PTPRC | 0.000058 |
| AA | SNP_A-4218063 | rs10919593 | 1 | 197077995 | downstream | 16630 | PTPRC | 0.000058 |
| CT | SNP_A-8682320 | rs12838423 | X | 4482942 | downstream | 1335142 | NLGN4X | 0.000208 |
| GT | SNP_A-8456832 | rs12857956 | X | 4452855 | downstream | 1365229 | NLGN4X | 0.000208 |
| AG | SNP_A-8548351 | rs5912192 | X | 78031569 | upstream | 55915 | P2RY10 | 0.000004 |
| AG | SNP_A-8548351 | rs5912192 | X | 78031569 | downstream | 132337 | LPAR4 | 0.000004 |
| AG | SNP_A-8548351 | rs5912192 | X | 78031569 | upstream | 55915 | P2RY10 | 0.000004 |
| AG | SNP_A-8548351 | rs5912192 | X | 78031569 | downstream | 132337 | LPAR4 | 0.000004 |
| CT | SNP_A-8682320 | rs12838423 | X | 4482942 | downstream | 1335142 | NLGN4X | 0.000208 |
| CT | SNP_A-8682320 | rs12838423 | X | 4482942 | downstream | 1335142 | NLGN4X | 0.000208 |
| GT | SNP_A-8456832 | rs12857956 | X | 4452855 | downstream | 1365229 | NLGN4X | 0.000208 |
| GT | SNP_A-8456832 | rs12857956 | X | 4452855 | downstream | 1365229 | NLGN4X | 0.000208 |
| AG | SNP_A-8548351 | rs5912192 | X | 78031569 | upstream | 55915 | P2RY10 | 0.000004 |
| AG | SNP_A-8548351 | rs5912192 | X | 78031569 | downstream | 132337 | LPAR4 | 0.000004 |
| AG | SNP_A-8548351 | rs5912192 | X | 78031569 | upstream | 55915 | P2RY10 | 0.000004 |
| AG | SNP_A-8548351 | rs5912192 | X | 78031569 | downstream | 132337 | LPAR4 | 0.000004 |
| AA | SNP_A-2294709 | rs3749796 | 5 | 179653982 | downstream | 6314 | GFPT2 | 0.000867 |
| AA | SNP_A-2294709 | rs3749796 | 5 | 179653982 | upstream | 13764 | MAPK9 | 0.000867 |
| TT | SNP_A-4262900 | rs17003277 | 21 | 14782024 | intron | 0 | SAMSN1 | 0.000619 |
| TT | SNP_A-4262900 | rs17003277 | 21 | 14782024 | intron | 0 | SAMSN1 | 0.000619 |
| TT | SNP_A-4262900 | rs17003277 | 21 | 14782024 | intron | 0 | SAMSN1 | 0.000619 |
| TT | SNP_A-4262900 | rs17003277 | 21 | 14782024 | intron | 0 | SAMSN1 | 0.000619 |
| AA | SNP_A-2294709 | rs3749796 | 5 | 179653982 | downstream | 6314 | GFPT2 | 0.000312 |
| AA | SNP_A-2294709 | rs3749796 | 5 | 179653982 | upstream | 13764 | MAPK9 | 0.000312 |
| AA | SNP_A-8299128 | rs1257689 | 14 | 98580183 | downstream | 53375 | BCL11B | 0.000765 |
| CC | SNP_A-8554692 | rs11030746 | 11 | 29592649 | upstream | 106940 | METT5D1 | 0.000423 |
| GG | SNP_A-4282690 | rs17120428 | 1 | 60494495 | upstream | 609024 | NFIA | 0.000487 |
| TT | SNP_A-2294709 | rs3749796 | 5 | 179653982 | downstream | 6314 | GFPT2 | 0.000211 |
| TT | SNP_A-2294709 | rs3749796 | 5 | 179653982 | upstream | 13764 | MAPK9 | 0.000211 |
| TT | SNP_A-8554692 | rs11030746 | 11 | 29592649 | upstream | 106940 | METT5D1 | 0.000472 |
| AA | SNP_A-2294709 | rs3749796 | 5 | 179653982 | downstream | 6314 | GFPT2 | 0.000312 |
| AA | SNP_A-2294709 | rs3749796 | 5 | 179653982 | upstream | 13764 | MAPK9 | 0.000312 |
| AA | SNP_A-8299128 | rs1257689 | 14 | 98580183 | downstream | 53375 | BCL11B | 0.000765 |
| CC | SNP_A-8554692 | rs11030746 | 11 | 29592649 | upstream | 106940 | METT5D1 | 0.000423 |
| GG | SNP_A-4282690 | rs17120428 | 1 | 60494495 | upstream | 609024 | NFIA | 0.000487 |
| TT | SNP_A-2294709 | rs3749796 | 5 | 179653982 | downstream | 6314 | GFPT2 | 0.000211 |
| TT | SNP_A-2294709 | rs3749796 | 5 | 179653982 | upstream | 13764 | MAPK9 | 0.000211 |
| TT | SNP_A-8554692 | rs11030746 | 11 | 29592649 | upstream | 106940 | METT5D1 | 0.000472 |
| AA | SNP_A-2294709 | rs3749796 | 5 | 179653982 | downstream | 6314 | GFPT2 | 0.000061 |
| AA | SNP_A-2294709 | rs3749796 | 5 | 179653982 | upstream | 13764 | MAPK9 | 0.000061 |
| AA | SNP_A-4282690 | rs17120428 | 1 | 60494495 | upstream | 609024 | NFIA | 0.000657 |
| AA | SNP_A-8301485 | rs16902320 | 8 | 128779894 | upstream | 37604 | MYC | 0.000924 |
| CC | SNP_A-4238494 | rs2862274 | 1 | 175945277 | downstream | 427096 | FAM5B | 0.000082 |
| CC | SNP_A-4262900 | rs17003277 | 21 | 14782024 | intron | 0 | SAMSN1 | 0.000090 |
| CC | SNP_A-4296112 | rs11103184 | 9 | 137811533 | intron | 0 | KCNT1 | 0.000194 |
| CC | SNP_A-8554692 | rs11030746 | 11 | 29592649 | upstream | 106940 | METT5D1 | 0.000020 |
| CC | SNP_A-8592516 | rs17078925 | 6 | 149213263 | intron | 0 | UST | 0.000382 |
| CC | SNP_A-8716016 | rs3816612 | 15 | 66438234 | intron | 0 | ITGA11 | 0.000122 |
| GG | SNP_A-4282690 | rs17120428 | 1 | 60494495 | upstream | 609024 | NFIA | 0.000317 |
| GG | SNP_A-8478767 | rs4595891 | 18 | 62571504 | upstream | 149308 | CDH19 | 0.000272 |
| TT | SNP_A-2294709 | rs3749796 | 5 | 179653982 | downstream | 6314 | GFPT2 | 0.000030 |
| TT | SNP_A-2294709 | rs3749796 | 5 | 179653982 | upstream | 13764 | MAPK9 | 0.000030 |
| TT | SNP_A-4238494 | rs2862274 | 1 | 175945277 | downstream | 427096 | FAM5B | 0.000075 |
| TT | SNP_A-4262900 | rs17003277 | 21 | 14782024 | intron | 0 | SAMSN1 | 0.000075 |
| TT | SNP_A-4296112 | rs11103184 | 9 | 137811533 | intron | 0 | KCNT1 | 0.000285 |
| TT | SNP_A-8478767 | rs4595891 | 18 | 62571504 | upstream | 149308 | CDH19 | 0.000365 |
| TT | SNP_A-8554692 | rs11030746 | 11 | 29592649 | upstream | 106940 | METT5D1 | 0.000021 |
| TT | SNP_A-8592516 | rs17078925 | 6 | 149213263 | intron | 0 | UST | 0.000576 |
| TT | SNP_A-8716016 | rs3816612 | 15 | 66438234 | intron | 0 | ITGA11 | 0.000103 |
| GG | SNP_A-4218063 | rs10919593 | 1 | 197077995 | downstream | 16630 | PTPRC | 0.000690 |
| GG | SNP_A-4218063 | rs10919593 | 1 | 197077995 | downstream | 16630 | PTPRC | 0.000690 |
| GG | SNP_A-42 18063 | rs10919593 | 1 | 197077995 | downstream | 16630 | PTPRC | 0.000690 |
| GG | SNP_A-4218063 | rs10919593 | 1 | 197077995 | downstream | 16630 | PTPRC | 0.000690 |
| TT | SNP_A-8716016 | rs3816612 | 15 | 66438234 | intron | 0 | ITGA11 | 0.000073 |
| AG | SNP_A-2267424 | rs5981167 | X | 68251184 | downstream | 46238 | PJA1 | 0.000738 |
| AG | SNP_A-2267424 | rs5981167 | X | 68251184 | downstream | 46238 | PJA1 | 0.000738 |
| AG | SNP_A-2267424 | rs5981167 | X | 68251184 | downstream | 272455 | EFNB1 | 0.000738 |
| AG | SNP_A-2267424 | rs5981167 | X | 68251184 | downstream | 272455 | EFNB1 | 0.000738 |
| GG | SNP_A-2267424 | rs5981167 | X | 68251184 | downstream | 46238 | PJA1 | 0.000665 |
| GG | SNP_A-2267424 | rs5981167 | X | 68251184 | downstream | 46238 | PJA1 | 0.000665 |
| GG | SNP_A-2267424 | rs5981167 | X | 68251184 | downstream | 272455 | EFNB1 | 0.000665 |
| GG | SNP_A-2267424 | rs5981167 | X | 68251184 | downstream | 272455 | EFNB1 | 0.000665 |
| CC | SNP_A-4262900 | rs17003277 | 21 | 14782024 | intron | 0 | SAMSN1 | 0.000702 |
| CC | SNP_A-4262900 | rs17003277 | 21 | 14782024 | intron | 0 | SAMSN1 | 0.000702 |
| TT | SNP_A-4262900 | rs17003277 | 21 | 14782024 | intron | 0 | SAMSN1 | 0.000852 |
| TT | SNP_A-4262900 | rs17003277 | 21 | 14782024 | intron | 0 | SAMSN1 | 0.000852 |
| CC | SNP_A-4262900 | rs17003277 | 21 | 14782024 | intron | 0 | SAMSN1 | 0.000702 |
| CC | SNP_A-4262900 | rs17003277 | 21 | 14782024 | intron | 0 | SAMSN1 | 0.000702 |
| TT | SNP_A-4262900 | rs17003277 | 21 | 14782024 | intron | 0 | SAMSN1 | 0.000852 |
| TT | SNP_A-4262900 | rs17003277 | 21 | 14782024 | intron | 0 | SAMSN1 | 0.000852 |
| CC | SNP_A-8717502 | rs536789 | X | 78087017 | upstream | 467 | P2RY10 | 0.000006 |
| CC | SNP_A-8717502 | rs536789 | X | 78087017 | upstream | 467 | P2RY10 | 0.000006 |
| CC | SNP_A-8717502 | rs536789 | X | 78087017 | downstream | 187785 | LPAR4 | 0.000006 |
| CC | SNP_A-8717502 | rs536789 | X | 78087017 | downstream | 187785 | LPAR4 | 0.000006 |
| GG | SNP_A-8281027 | rs496666 | X | 78063163 | upstream | 24321 | P2RY10 | 0.000005 |
| GG | SNP_A-8281027 | rs496666 | X | 78063163 | upstream | 24321 | P2RY10 | 0.000005 |
| GG | SNP_A-8281027 | rs496666 | X | 78063163 | downstream | 163931 | LPAR4 | 0.000005 |
| GG | SNP_A-8281027 | rs496666 | X | 78063163 | downstream | 163931 | LPAR4 | 0.000005 |
| GG | SNP_A-8548351 | rs5912192 | X | 78031569 | upstream | 55915 | P2RY10 | 0.000009 |
| GG | SNP_A-8548351 | rs5912192 | X | 78031569 | upstream | 55915 | P2RY10 | 0.000009 |
| GG | SNP_A-8548351 | rs5912192 | X | 78031569 | downstream | 132337 | LPAR4 | 0.000009 |
| GG | SNP_A-8548351 | rs5912192 | X | 78031569 | downstream | 132337 | LPAR4 | 0.000009 |
| TT | SNP_A-8546758 | rs7063827 | X | 78241380 | upstream | 71744 | GPR174 | 0.000006 |
| TT | SNP_A-8546758 | rs7063827 | X | 78241380 | upstream | 71744 | GPR174 | 0.000006 |
| TT | SNP_A-8546758 | rs7063827 | X | 78241380 | downstream | 137287 | P2RY10 | 0.000006 |
| TT | SNP_A-8546758 | rs7063827 | X | 78241380 | downstream | 137287 | P2RY10 | 0.000006 |
| CC | SNP_A-4238494 | rs2862274 | 1 | 175945277 | downstream | 427096 | FAM5B | 0.000932 |
| CC | SNP_A-4238494 | rs2862274 | 1 | 175945277 | downstream | 427096 | FAM5B | 0.000932 |
| TT | SNP_A-4238494 | rs2862274 | 1 | 175945277 | downstream | 427096 | FAM5B | 0.000015 |
| TT | SNP_A-4238494 | rs2862274 | 1 | 175945277 | downstream | 427096 | FAM5B | 0.000015 |
| CC | SNP_A-4238494 | rs2862274 | 1 | 175945277 | downstream | 427096 | FAM5B | 0.000932 |
| CC | SNP_A-4238494 | rs2862274 | 1 | 175945277 | downstream | 427096 | FAM5B | 0.000932 |
| TT | SNP_A-4238494 | rs2862274 | 1 | 175945277 | downstream | 427096 | FAM5B | 0.000015 |
| TT | SNP_A-4238494 | rs2862274 | 1 | 175945277 | downstream | 427096 | FAM5B | 0.000015 |
| AA | SNP_A-2294709 | rs3749796 | 5 | 179653982 | downstream | 6314 | GFPT2 | 0.000133 |
| AA | SNP_A-2294709 | rs3749796 | 5 | 179653982 | upstream | 13764 | MAPK9 | 0.000133 |
| AA | SNP_A-2294709 | rs3749796 | 5 | 179653982 | downstream | 6314 | GFPT2 | 0.000133 |
| AA | SNP_A-2294709 | rs3749796 | 5 | 179653982 | upstream | 13764 | MAPK9 | 0.000133 |
| AA | SNP_A-2294709 | rs3749796 | 5 | 179653982 | downstream | 6314 | GFPT2 | 0.000154 |
| AA | SNP_A-2294709 | rs3749796 | 5 | 179653982 | upstream | 13764 | MAPK9 | 0.000154 |
| CC | SNP_A-4238494 | rs2862274 | 1 | 175945277 | downstream | 427096 | FAM5B | 0.000036 |
| TT | SNP_A-4238494 | rs2862274 | 1 | 175945277 | downstream | 427096 | FAM5B | 0.000033 |
| TT | SNP_A-8300818 | rs2274703 | 1 | 203620091 | intron | 0 | LEMD1 | 0.000007 |
| AA | SNP_A-2294709 | rs3749796 | 5 | 179653982 | downstream | 6314 | GFPT2 | 0.000154 |
| AA | SNP_A-2294709 | rs3749796 | 5 | 179653982 | upstream | 13764 | MAPK9 | 0.000154 |
| CC | SNP_A-4238494 | rs2862274 | 1 | 175945277 | downstream | 427096 | FAM5B | 0.000036 |
| TT | SNP_A-4238494 | rs2862274 | 1 | 175945277 | downstream | 427096 | FAM5B | 0.000033 |
| AA | SNP_A-2107652 | rs3925518 | 5 | 178004835 | upstream | 18175 | CLK4 | 0.000666 |
| AA | SNP_A-2107652 | rs3925518 | 5 | 178004835 | downstream | 66292 | ZNF354A | 0.000666 |
| AA | SNP_A-2294709 | rs3749796 | 5 | 179653982 | upstream | 2305 | MAPK9 | 0.000361 |
| AA | SNP_A-2294709 | rs3749796 | 5 | 179653982 | downstream | 6323 | GFPT2 | 0.000361 |
| AA | SNP_A-4218063 | rs10919593 | 1 | 197077995 | downstream | 84827 | PTPRC | 0.000138 |
| AA | SNP_A-4218063 | rs10919593 | 1 | 197077995 | upstream | 106760 | RPS2P9 | 0.000138 |
| AA | SNP_A-8347387 | rs2525841 | 12 | 80054239 | intron | 0 | ACSS3 | 0.000481 |
| AG | SNP_A-8446732 | rs17154320 | 7 | 80149730 | downstream | 60060 | SEMA3C | 0.000292 |
| AT | SNP_A-8703223 | rs6641231 | X | 147345728 | upstream | 8164 | RPL7P57 | 0.000280 |
| AT | SNP_A-8703223 | rs6641231 | X | 147345728 | upstream | 257537 | HAX1 | 0.000280 |
| CC | SNP_A-1797721 | rs11183915 | 12 | 46057251 | upstream | 515 | LOC728148 | 0.000474 |
| CC | SNP_A-4296112 | rs11103184 | 9 | 137811533 | intron | 0 | KCNT1 | 0.000102 |
| CC | SNP_A-8475711 | rs7827228 | 8 | 2577400 | downstream | 202881 | CSMD1 | 0.000777 |
| CC | SNP_A-8475711 | rs7827228 | 8 | 2577400 | downstream | 496614 | MYOM2 | 0.000777 |
| CC | SNP_A-8691130 | rs11103512 | 9 | 136796458 | intron | 0 | COL5A1 | 0.000059 |
| CG | SNP_A-8339688 | rs6540375 | X | 147371489 | downstream | 16849 | RPL7P57 | 0.000485 |
| CG | SNP_A-8339688 | rs6540375 | X | 147371489 | upstream | 18341 | AFF2 | 0.000485 |
| CT | SNP_A-8339687 | rs6419449 | X | 147361392 | downstream | 6752 | RPL7P57 | 0.000527 |
| CT | SNP_A-8339687 | rs6419449 | X | 147361392 | upstream | 28438 | AFF2 | 0.000527 |
| CT | SNP_A-8449633 | rs9586459 | 13 | 103750145 | upstream | 1166219 | DAOA | 0.000007 |
| GG | SNP_A-4218063 | rs10919593 | 1 | 197077995 | downstream | 84827 | PTPRC | 0.000510 |
| GG | SNP_A-4218063 | rs10919593 | 1 | 197077995 | upstream | 106760 | RPS2P9 | 0.000510 |
| TT | SNP_A-8449633 | rs9586459 | 13 | 103750145 | upstream | 1166219 | DAOA | 0.000007 |
| TT | SNP_A-8494779 | rs6077004 | 20 | 6390961 | upstream | 246605 | LOC728383 | 0.000170 |
| TT | SNP_A-8494779 | rs6077004 | 20 | 6390961 | upstream | 260123 | TARDBPL | 0.000170 |
| TT | SNP_A-8619237 | rs1822910 | 3 | 146474560 | downstream | 547087 | RPL21P46 | 0.000214 |
| AA | SNP_A-2294709 | rs3749796 | 5 | 179653982 | downstream | 6314 | GFPT2 | 0.000361 |
| AA | SNP_A-2294709 | rs3749796 | 5 | 179653982 | upstream | 13764 | MAPK9 | 0.000361 |
| AA | SNP_A-4218063 | rs10919593 | 1 | 197077995 | downstream | 16630 | PTPRC | 0.000138 |
| CC | SNP_A-4296112 | rs11103184 | 9 | 137811533 | intron | 0 | KCNT1 | 0.000102 |
| GG | SNP_A-4218063 | rs10919593 | 1 | 197077995 | downstream | 16630 | PTPRC | 0.000510 |
| AA | SNP_A-2294709 | rs3749796 | 5 | 179653982 | downstream | 6314 | GFPT2 | 0.000361 |
| AA | SNP_A-2294709 | rs3749796 | 5 | 179653982 | upstream | 13764 | MAPK9 | 0.000361 |
| AA | SNP_A-4218063 | rs10919593 | 1 | 197077995 | downstream | 16630 | PTPRC | 0.000138 |
| CC | SNP_A-4296112 | rs11103184 | 9 | 137811533 | intron | 0 | KCNT1 | 0.000102 |
| CT | SNP_A-8449633 | rs9586459 | 13 | 103750145 | upstream | 1166219 | DAOA | 0.000007 |
| GG | SNP_A-4218063 | rs10919593 | 1 | 197077995 | downstream | 16630 | PTPRC | 0.000510 |
| TT | SNP_A-8449633 | rs9586459 | 13 | 103750145 | upstream | 1166219 | DAOA | 0.000007 |
| AA | SNP_A-4218063 | rs10919593 | 1 | 197077995 | downstream | 84827 | PTPRC | 0.000005 |
| AA | SNP_A-4218063 | rs10919593 | 1 | 197077995 | upstream | 106760 | RPS2P9 | 0.000005 |
| TT | SNP_A-4238494 | rs2862274 | 1 | 175945277 | intron | 0 | LOC400796 | 0.000007 |
| AA | SNP_A-4218063 | rs10919593 | 1 | 197077995 | downstream | 84827 | PTPRC | 0.000005 |
| AA | SNP_A-4218063 | rs10919593 | 1 | 197077995 | upstream | 106760 | RPS2P9 | 0.000005 |
| TT | SNP_A-4238494 | rs2862274 | 1 | 175945277 | intron | 0 | LOC400796 | 0.000007 |
| AA | SNP_A-2294709 | rs3749796 | 5 | 179653982 | upstream | 2305 | MAPK9 | 0.000966 |
| AA | SNP_A-2294709 | rs3749796 | 5 | 179653982 | downstream | 6323 | GFPT2 | 0.000966 |
| CT | SNP_A-1998103 | rs3859965 | X | 3959212 | downstream | 1858872 | NLGN4X | 0.000500 |
| CT | SNP_A-1998103 | rs3859965 | X | 3959212 | downstream | 1858872 | NLGN4X | 0.000500 |
| AC | SNP_A-8672156 | rs10227794 | 7 | 52274133 | upstream | 796709 | POM121L12 | 0.000009 |
| CC | SNP_A-8672156 | rs10227794 | 7 | 52274133 | upstream | 796709 | POM121L12 | 0.000009 |
| CG | SNP_A-1881982 | rs2237342 | 7 | 27762767 | intron | — | TAX1BP1 | 0.000663 |
| CG | SNP_A-1881982 | rs2237342 | 7 | 27762767 | intron | — | TAX1BP1 | 0.000663 |
| GT | SNP_A-1831539 | rs2144497 | X | 32315082 | intron | 0 | DMD | 0.000000 |
| GT | SNP_A-1831539 | rs2144497 | X | 32315082 | intron | 0 | DMD | 0.000000 |
| AA | SNP_A-4218063 | rs10919593 | 1 | 197077995 | downstream | 16630 | PTPRC | 0.000005 |
| AA | SNP_A-8301485 | rs16902320 | 8 | 128779894 | upstream | 37604 | MYC | 0.000495 |
| CC | SNP_A-4238494 | rs2862274 | 1 | 175945277 | downstream | 427096 | FAM5B | 0.000332 |
| CC | SNP_A-8301485 | rs16902320 | 8 | 128779894 | upstream | 37604 | MYC | 0.000316 |
| TT | SNP_A-2294709 | rs3749796 | 5 | 179653982 | downstream | 6314 | GFPT2 | 0.000391 |
| TT | SNP_A-2294709 | rs3749796 | 5 | 179653982 | upstream | 13764 | MAPK9 | 0.000391 |
| TT | SNP_A-4238494 | rs2862274 | 1 | 175945277 | downstream | 427096 | FAM5B | 0.000007 |
| TT | SNP_A-8554692 | rs11030746 | 11 | 29592649 | upstream | 106940 | METT5D1 | 0.000471 |
| AA | SNP_A-4218063 | rs10919593 | 1 | 197077995 | downstream | 16630 | PTPRC | 0.000005 |
| AA | SNP_A-8301485 | rs16902320 | 8 | 128779894 | upstream | 37604 | MYC | 0.000495 |
| CC | SNP_A-4238494 | rs2862274 | 1 | 175945277 | downstream | 427096 | FAM5B | 0.000332 |
| CC | SNP_A-8301485 | rs16902320 | 8 | 128779894 | upstream | 37604 | MYC | 0.000316 |
| TT | SNP_A-2294709 | rs3749796 | 5 | 179653982 | downstream | 6314 | GFPT2 | 0.000391 |
| TT | SNP_A-2294709 | rs3749796 | 5 | 179653982 | upstream | 13764 | MAPK9 | 0.000391 |
| TT | SNP_A-4238494 | rs2862274 | 1 | 175945277 | downstream | 427096 | FAM5B | 0.000007 |
| TT | SNP_A-8554692 | rs11030746 | 11 | 29592649 | upstream | 106940 | METT5D1 | 0.000471 |
| CC | SNP_A-8554692 | rs11030746 | 11 | 29592649 | upstream | 106940 | METT5D1 | 0.000034 |
| CC | SNP_A-8554692 | rs11030746 | 11 | 29592649 | upstream | 106940 | METT5D1 | 0.000034 |
| CC | SNP_A-8716016 | rs3816612 | 15 | 66438234 | intron | 0 | ITGA11 | 0.000908 |
| CC | SNP_A-8716016 | rs3816612 | 15 | 66438234 | intron | 0 | ITGA11 | 0.000908 |
| TT | SNP_A-2294709 | rs3749796 | 5 | 179653982 | downstream | 6314 | GFPT2 | 0.000060 |
| TT | SNP_A-2294709 | rs3749796 | 5 | 179653982 | downstream | 6314 | GFPT2 | 0.000060 |
| TT | SNP_A-2294709 | rs3749796 | 5 | 179653982 | upstream | 13764 | MAPK9 | 0.000060 |
| TT | SNP_A-2294709 | rs3749796 | 5 | 179653982 | upstream | 13764 | MAPK9 | 0.000060 |
| TT | SNP_A-8554692 | rs11030746 | 11 | 29592649 | upstream | 106940 | METT5D1 | 0.000024 |
| TT | SNP_A-8554692 | rs11030746 | 11 | 29592649 | upstream | 106940 | METT5D1 | 0.000024 |
| CC | SNP_A-4238494 | rs2862274 | 1 | 175945277 | downstream | 427096 | FAM5B | 0.000727 |
| CC | SNP_A-4238494 | rs2862274 | 1 | 175945277 | downstream | 427096 | FAM5B | 0.000727 |
| TT | SNP_A-4238494 | rs2862274 | 1 | 175945277 | downstream | 427096 | FAM5B | 0.000435 |
| TT | SNP_A-4238494 | rs2862274 | 1 | 175945277 | downstream | 427096 | FAM5B | 0.000435 |
| CC | SNP_A-4238494 | rs2862274 | 1 | 175945277 | downstream | 427096 | FAM5B | 0.000727 |
| CC | SNP_A-4238494 | rs2862274 | 1 | 175945277 | downstream | 427096 | FAM5B | 0.000727 |
| TT | SNP_A-4238494 | rs2862274 | 1 | 175945277 | downstream | 427096 | FAM5B | 0.000435 |
| TT | SNP_A-4238494 | rs2862274 | 1 | 175945277 | downstream | 427096 | FAM5B | 0.000435 |
| AA | SNP_A-8634376 | rs2623149 | 8 | 95309158 | downstream | 21504 | GEM | 0.000502 |
| AC | SNP_A-8388931 | rs11625236 | 14 | 57344492 | upstream | 392093 | ACTR10 | 0.000857 |
| AG | SNP_A-8634376 | rs2623149 | 8 | 95309158 | downstream | 21504 | GEM | 0.000502 |
| CC | SNP_A-8300818 | rs2274703 | 1 | 203620091 | intron | 0 | LEMD1 | 0.000810 |
| CT | SNP_A-8485365 | rs2613216 | 8 | 95312238 | downstream | 18424 | GEM | 0.000502 |
| TT | SNP_A-8485365 | rs2613216 | 8 | 95312238 | downstream | 18424 | GEM | 0.000502 |
| CC | SNP_A-4238494 | rs2862274 | 1 | 175945277 | downstream | 427096 | FAM5B | 0.000829 |
| CC | SNP_A-4238494 | rs2862274 | 1 | 175945277 | downstream | 427096 | FAM5B | 0.000829 |
| TT | SNP_A-4238494 | rs2862274 | 1 | 175945277 | downstream | 427096 | FAM5B | 0.000751 |
| TT | SNP_A-4238494 | rs2862274 | 1 | 175945277 | downstream | 427096 | FAM5B | 0.000751 |
| CC | SNP_A-4238494 | rs2862274 | 1 | 175945277 | downstream | 427096 | FAM5B | 0.000829 |
| CC | SNP_A-4238494 | rs2862274 | 1 | 175945277 | downstream | 427096 | FAM5B | 0.000829 |
| TT | SNP_A-4238494 | rs2862274 | 1 | 175945277 | downstream | 427096 | FAM5B | 0.000751 |
| TT | SNP_A-4238494 | rs2862274 | 1 | 175945277 | downstream | 427096 | FAM5B | 0.000751 |
| AA | SNP_A-8347387 | rs2525841 | 12 | 80054239 | intron | 0 | ACSS3 | 0.000921 |
| AG | SNP_A-8446732 | rs17154320 | 7 | 80149730 | downstream | 60060 | SEMA3C | 0.000809 |
| AA | SNP_A-2294709 | rs3749796 | 5 | 179653982 | downstream | 6314 | GFPT2 | 0.000966 |
| AA | SNP_A-2294709 | rs3749796 | 5 | 179653982 | upstream | 13764 | MAPK9 | 0.000966 |

TABLE 9
Characteristics of Longevity Interactions Affecting More Than One Buffered and Buffering Gene

| | | | Chr- | Physical | Geno- | | | | Physical | |
|---|---|---|---|---|---|---|---|---|---|---|
| Geno- | | dbSNP- | Buff- | Position- | Buff- | | dbSNP- | Chr- | position- | |
| Buffering | Buffering | Buffering | ering | Buffering | ered | Buffered | Buffered | Buffered | Buffered | P-value |
| AA | SNP_A-2083501 | rs4686870 | 3 | 188500535 | AA | SNP_A-2294709 | rs3749796 | 5 | 179653982 | 2.38E−05 |
| AA | SNP_A-4180725 | rs7106605 | 11 | 103045752 | AA | SNP_A-2294709 | rs3749796 | 5 | 179653982 | 0.00026015 |
| GG | SNP_A-4180725 | rs7106605 | 11 | 103045752 | AA | SNP_A-2294709 | rs3749796 | 5 | 179653982 | 0.00026015 |
| CT | SNP_A-4291022 | rs5979604 | X | 12274581 | AA | SNP_A-2294709 | rs3749796 | 5 | 179653982 | 0.00086661 |
| AA | SNP_A-8605897 | rs7130910 | 11 | 103050448 | AA | SNP_A-2294709 | rs3749796 | 5 | 179653982 | 0.00033226 |
| GG | SNP_A-8605897 | rs7130910 | 11 | 103050448 | AA | SNP_A-2294709 | rs3749796 | 5 | 179653982 | 0.00033226 |
| CT | SNP_A-8665678 | rs11738547 | 5 | 10087081 | AA | SNP_A-2294709 | rs3749796 | 5 | 179653982 | 0.00031194 |
| GG | SNP_A-1854119 | rs7221263 | 17 | 39367221 | AA | SNP_A-2294709 | rs3749796 | 5 | 179653982 | 6.10E−05 |
| GG | SNP_A-8574340 | rs7579939 | 2 | 51842138 | AA | SNP_A-2294709 | rs3749796 | 5 | 179653982 | 0.00013315 |
| GG | SNP_A-8580357 | rs1430707 | 2 | 131282490 | AA | SNP_A-2294709 | rs3749796 | 5 | 179653982 | 0.00015401 |
| TT | SNP_A-8673617 | rs2615903 | 11 | 33414403 | AA | SNP_A-2294709 | rs3749796 | 5 | 179653982 | 0.00096618 |
| GG | SNP_A-8687186 | rs17014445 | 2 | 34058603 | AA | SNP_A-2294709 | rs3749796 | 5 | 179653982 | 0.00036057 |
| AA | SNP_A-2083501 | rs4686870 | 3 | 188500535 | AA | SNP_A-4218063 | rs10919593 | 1 | 197077995 | 1.67E−05 |
| GG | SNP_A-8374392 | rs6066159 | 20 | 45074038 | AA | SNP_A-4218063 | rs10919593 | 1 | 197077995 | 0.00057585 |
| CG | SNP_A-8374392 | rs6066159 | 20 | 45074038 | AA | SNP_A-4218063 | rs10919593 | 1 | 197077995 | 0.00057585 |
| CC | SNP_A-8452641 | rs1499953 | 3 | 116719791 | AA | SNP_A-4218063 | rs10919593 | 1 | 197077995 | 5.81E−05 |
| TT | SNP_A-8438227 | rs4686457 | 3 | 188502086 | AA | SNP_A-4218063 | rs10919593 | 1 | 197077995 | 4.58E−06 |
| GG | SNP_A-8687186 | rs17014445 | 2 | 34058603 | AA | SNP_A-4218063 | rs10919593 | 1 | 197077995 | 0.00013795 |
| GG | SNP_A-1854119 | rs7221263 | 17 | 39367221 | AA | SNP_A-4282690 | rs17120428 | 1 | 60494495 | 0.00065701 |
| CC | SNP_A-8632732 | rs999430 | 17 | 73983274 | CC | SNP_A-8300818 | rs2274703 | 1 | 203620091 | 0.00011148 |
| TT | SNP_A-2298975 | rs12276840 | 11 | 131853608 | CC | SNP_A-8300818 | rs2274703 | 1 | 203620091 | 0.00033637 |
| CC | SNP_A-2298975 | rs12276840 | 11 | 131853608 | CC | SNP_A-8300818 | rs2274703 | 1 | 203620091 | 0.00033637 |
| GG | SNP_A-1854119 | rs7221263 | 17 | 39367221 | CC | SNP_A-8300818 | rs2274703 | 1 | 203620091 | 0.00014117 |
| GG | SNP_A-8574340 | rs7579939 | 2 | 51842138 | CC | SNP_A-8300818 | rs2274703 | 1 | 203620091 | 0.00031348 |
| TT | SNP_A-8619237 | rs1822910 | 3 | 146474560 | CC | SNP_A-8300818 | rs2274703 | 1 | 203620091 | 0.00080982 |
| GG | SNP_A-1854119 | rs7221263 | 17 | 39367221 | CC | SNP_A-8673617 | rs2615903 | 11 | 33414403 | 0.00018911 |
| GG | SNP_A-1905146 | rs2012129 | 15 | 97950440 | TT | SNP_A-2294709 | rs3749796 | 5 | 179653982 | 0.00012279 |
| CG | SNP_A-1905146 | rs2012129 | 15 | 97950440 | TT | SNP_A-2294709 | rs3749796 | 5 | 179653982 | 0.00012279 |
| AA | SNP_A-2083501 | rs4686870 | 3 | 188500535 | TT | SNP_A-2294709 | rs3749796 | 5 | 179653982 | 0.00081455 |
| TT | SNP_A-2310343 | rs325380 | 15 | 98074141 | TT | SNP_A-2294709 | rs3749796 | 5 | 179653982 | 0.00009741 |
| AT | SNP_A-2310343 | rs325380 | 15 | 98074141 | TT | SNP_A-2294709 | rs3749796 | 5 | 179653982 | 0.00009741 |
| GG | SNP_A-4289735 | rs2061007 | 15 | 98087203 | TT | SNP_A-2294709 | rs3749796 | 5 | 179653982 | 9.87E−05 |
| CG | SNP_A-4289735 | rs2061007 | 15 | 98087203 | TT | SNP_A-2294709 | rs3749796 | 5 | 179653982 | 9.87E−05 |
| CT | SNP_A-8665678 | rs11738547 | 5 | 10087081 | TT | SNP_A-2294709 | rs3749796 | 5 | 179653982 | 0.00021055 |
| GG | SNP_A-1854119 | rs7221263 | 17 | 39367221 | TT | SNP_A-2294709 | rs3749796 | 5 | 179653982 | 3.03E−05 |
| TT | SNP_A-8438227 | rs4686457 | 3 | 188502086 | TT | SNP_A-2294709 | rs3749796 | 5 | 179653982 | 0.00039138 |
| AA | SNP_A-8453929 | rs1991921 | 15 | 98011512 | TT | SNP_A-2294709 | rs3749796 | 5 | 179653982 | 5.97E−05 |
| AT | SNP_A-8453929 | rs1991921 | 15 | 98011512 | TT | SNP_A-2294709 | rs3749796 | 5 | 179653982 | 5.97E−05 |
| TT | SNP_A-8687186 | rs17014445 | 2 | 34058603 | GG | SNP_A-4218063 | rs10919593 | 1 | 197077995 | 0.0008072 |
| AT | SNP_A-8687186 | rs17014445 | 2 | 34058603 | GG | SNP_A-4218063 | rs10919593 | 1 | 197077995 | 0.0008072 |
| CC | SNP_A-1996154 | rs10738555 | 9 | 19915073 | GG | SNP_A-4218063 | rs10919593 | 1 | 197077995 | 0.00069018 |
| CG | SNP_A-1996154 | rs10738555 | 9 | 19915073 | GG | SNP_A-4218063 | rs10919593 | 1 | 197077995 | 0.00069018 |
| GG | SNP_A-8687186 | rs17014445 | 2 | 34058603 | GG | SNP_A-4218063 | rs10919593 | 1 | 197077995 | 0.00050999 |
| TT | SNP_A-4297368 | rs6851158 | 4 | 113998568 | GG | SNP_A-4282690 | rs17120428 | 1 | 60494495 | 0.00067607 |
| AT | SNP_A-4297368 | rs6851158 | 4 | 113998568 | GG | SNP_A-4282690 | rs17120428 | 1 | 60494495 | 0.00067607 |
| CT | SNP_A-8665678 | rs11738547 | 5 | 10087081 | GG | SNP_A-4282690 | rs17120428 | 1 | 60494495 | 0.00048653 |
| GG | SNP_A-1854119 | rs7221263 | 17 | 39367221 | GG | SNP_A-4282690 | rs17120428 | 1 | 60494495 | 0.00031725 |
| GG | SNP_A-1935646 | rs2098846 | — | — | TT | SNP_A-8300818 | rs2274703 | 1 | 203620091 | 0.00061044 |
| CG | SNP_A-1935646 | rs2098846 | — | — | TT | SNP_A-8300818 | rs2274703 | 1 | 203620091 | 0.00061044 |
| GG | SNP_A-4289735 | rs2061007 | 15 | 98087203 | TT | SNP_A-8300818 | rs2274703 | 1 | 203620091 | 2.33E−05 |
| CG | SNP_A-4289735 | rs2061007 | 15 | 98087203 | TT | SNP_A-8300818 | rs2274703 | 1 | 203620091 | 2.33E−05 |
| CC | SNP_A-8632732 | rs999430 | 17 | 73983274 | TT | SNP_A-8300818 | rs2274703 | 1 | 203620091 | 0.000616 |
| GG | SNP_A-1854119 | rs7221263 | 17 | 39367221 | TT | SNP_A-8300818 | rs2274703 | 1 | 203620091 | 1.36E−05 |
| GG | SNP_A-8580357 | rs1430707 | 2 | 131282490 | TT | SNP_A-8300818 | rs2274703 | 1 | 203620091 | 6.84E−06 |
| TT | SNP_A-8673617 | rs2615903 | 11 | 33414403 | AA | SNP_A-2294709 | rs3749796 | 5 | 179653982 | 0.00096618 |
| TT | SNP_A-8673617 | rs2615903 | 11 | 33414403 | AA | SNP_A-8347387 | rs2525841 | 12 | 80054239 | 0.00092098 |
| AA | SNP_A-8300818 | rs2274703 | 1 | 203620091 | CG | SNP_A-1881982 | rs2237342 | 7 | 27762767 | 0.00023386 |
| AT | SNP_A-8300818 | rs2274703 | 1 | 203620091 | CG | SNP_A-1881982 | rs2237342 | 7 | 27762767 | 0.00023386 |
| TT | SNP_A-4218063 | rs10919593 | 1 | 197077995 | CT | SNP_A-8445436 | rs642053 | 1 | 217173829 | 0.00044651 |
| AT | SNP_A-4218063 | rs10919593 | 1 | 197077995 | CT | SNP_A-8445436 | rs642053 | 1 | 217173829 | 0.00044651 |
| TT | SNP_A-8673617 | rs2615903 | 11 | 33414403 | AG | SNP_A-8446732 | rs17154320 | 7 | 80149730 | 0.00080902 |
| CC | SNP_A-8639378 | rs12432648 | 14 | 39997127 | GT | SNP_A-8456832 | rs12857956 | X | 4452855 | 0.00020803 |
| CC | SNP_A-8639378 | rs12432648 | 14 | 39997127 | CT | SNP_A-8682320 | rs12838423 | X | 4482942 | 0.00020803 |
| TT | SNP_A-4218063 | rs10919593 | 1 | 197077995 | TT | SNP_A-8445436 | rs642053 | 1 | 217173829 | 0.0007686 |
| AT | SNP_A-4218063 | rs10919593 | 1 | 197077995 | TT | SNP_A-8445436 | rs642053 | 1 | 217173829 | 0.0007686 |
| CC | SNP_A-8300818 | rs2274703 | 1 | 203620091 | CC | SNP_A-8475711 | rs7827228 | 8 | 2577400 | 0.00048994 |

TABLE 10
Characteristics of Buffering Genotype Affecting Multiple Genes

| | | | | Physical | | | | |
|---|---|---|---|---|---|---|---|---|
| Geno- | | dbSNP- | Chr- | Position- | 1st Gene- | Position 1- | 2nd Gene- | Position 2- |
| Buffering | Buffering | Buffering | Buffering | Buffering | Buffering | Buffering | Buffering | Buffering |
| AA | SNP_A-2083501 | rs4686870 | 3 | 188500535 | RTP4 | upstream | MASP1 | upstream |
| AA | SNP_A-4180725 | rs7106605 | 11 | 103045752 | PDGFD | downstream | DYNC2H1 | downstream |
| GG | SNP_A-4180725 | rs7106605 | 11 | 103045752 | PDGFD | downstream | DYNC2H1 | downstream |
| CT | SNP_A-4291022 | rs5979604 | X | 12274581 | FRMPD4 | intron | | |
| AA | SNP_A-8605897 | rs7130910 | 11 | 103050448 | PDGFD | downstream | DYNC2H1 | downstream |
| GG | SNP_A-8605897 | rs7130910 | 11 | 103050448 | PDGFD | downstream | DYNC2H1 | downstream |
| CT | SNP_A-8665678 | rs11738547 | 5 | 10087081 | FAM173B | downstream | TAS2R1 | upstream |
| GG | SNP_A-1854119 | rs7221263 | 17 | 39367221 | PPY | downstream | | |
| GG | SNP_A-8574340 | rs7579939 | 2 | 51842138 | ASB3 | downstream | NRXN1 | upstream |
| GG | SNP_A-8580357 | rs1430707 | 2 | 131282490 | FAM123C | downstream | | |
| TT | SNP_A-8673617 | rs2615903 | 11 | 33414403 | HIPK3 | downstream | | |
| GG | SNP_A-8687186 | rs17014445 | 2 | 34058603 | SLC25A5 | upstream | | |
| AA | SNP_A-2083501 | rs4686870 | 3 | 188500535 | RTP4 | upstream | MASP1 | upstream |
| GG | SNP_A-8374392 | rs6066159 | 20 | 45074038 | EYA2 | intron | | |
| CG | SNP_A-8374392 | rs6066159 | 20 | 45074038 | EYA2 | intron | | |
| CC | SNP_A-8452641 | rs1499953 | 3 | 116719791 | GAP43 | upstream | ZBTB20 | upstream |
| TT | SNP_A-8438227 | rs4686457 | 3 | 188502086 | RTP4 | upstream | MASP1 | upstream |
| GG | SNP_A-8687186 | rs17014445 | 2 | 34058603 | SLC25A5 | upstream | | |
| GG | SNP_A-1854119 | rs7221263 | 17 | 39367221 | PPY | downstream | | |
| CC | SNP_A-8632732 | rs999430 | 17 | 73983274 | PGS1 | downstream | | |
| TT | SNP_A-2298975 | rs12276840 | 11 | 131853608 | OPCML | intron | | |
| CC | SNP_A-2298975 | rs12276840 | 11 | 131853608 | OPCML | intron | | |
| GG | SNP_A-1854119 | rs7221263 | 17 | 39367221 | PPY | downstream | | |
| GG | SNP_A-8574340 | rs7579939 | 2 | 51842138 | ASB3 | downstream | NRXN1 | upstream |
| TT | SNP_A-8619237 | rs1822910 | 3 | 146474560 | RPL21P46 | downstream | | |
| GG | SNP_A-1854119 | rs7221263 | 17 | 39367221 | PPY | downstream | | |
| GG | SNP_A-1905146 | rs2012129 | 15 | 97950440 | MEF2A | intron | | |
| CG | SNP_A-1905146 | rs2012129 | 15 | 97950440 | MEF2A | intron | | |
| AA | SNP_A-2083501 | rs4686870 | 3 | 188500535 | RTP4 | upstream | MASP1 | upstream |
| TT | SNP_A-2310343 | rs325380 | 15 | 98074141 | MEF2A | 3UTR | | |
| AT | SNP_A-2310343 | rs325380 | 15 | 98074141 | MEF2A | 3UTR | | |
| GG | SNP_A-4289735 | rs2061007 | 15 | 98087203 | LYSMD4 | CDS | | |
| CG | SNP_A-4289735 | rs2061007 | 15 | 98087203 | LYSMD4 | CDS | | |
| CT | SNP_A-8665678 | rs11738547 | 5 | 10087081 | FAM173B | downstream | TAS2R1 | upstream |
| GG | SNP_A-1854119 | rs7221263 | 17 | 39367221 | PPY | downstream | | |
| TT | SNP_A-8438227 | rs4686457 | 3 | 188502086 | RTP4 | upstream | MASP1 | upstream |
| AA | SNP_A-8453929 | rs1991921 | 15 | 98011512 | MEF2A | intron | | |
| AT | SNP_A-8453929 | rs1991921 | 15 | 98011512 | MEF2A | intron | | |
| TT | SNP_A-8687186 | rs17014445 | 2 | 34058603 | SLC25A5 | upstream | | |
| AT | SNP_A-8687186 | rs17014445 | 2 | 34058603 | SLC25A5 | upstream | | |
| CC | SNP_A-1996154 | rs10738555 | 9 | 19915073 | MLLT3 | downstream | SLC24A2 | upstream |
| CG | SNP_A-1996154 | rs10738555 | 9 | 19915073 | MLLT3 | downstream | SLC24A2 | upstream |
| GG | SNP_A-8687186 | rs17014445 | 2 | 34058603 | SLC25A5 | upstream | | |
| TT | SNP_A-4297368 | rs6851158 | 4 | 113998568 | ANK2 | intron | | |
| AT | SNP_A-4297368 | rs6851158 | 4 | 113998568 | ANK2 | intron | | |
| CT | SNP_A-8665678 | rs11738547 | 5 | 10087081 | FAM173B | downstream | TAS2R1 | upstream |
| GG | SNP_A-1854119 | rs7221263 | 17 | 39367221 | PPY | downstream | | |
| GG | SNP_A-1935646 | rs2098846 | — | — | — | — | | |
| CG | SNP_A-1935646 | rs2098846 | — | — | — | — | | |
| GG | SNP_A-4289735 | rs2061007 | 15 | 98087203 | LYSMD4 | CDS | | |
| CG | SNP_A-4289735 | rs2061007 | 15 | 98087203 | LYSMD4 | CDS | | |
| CC | SNP_A-8632732 | rs999430 | 17 | 73983274 | PGS1 | downstream | | |
| GG | SNP_A-1854119 | rs7221263 | 17 | 39367221 | PPY | downstream | | |
| GG | SNP_A-8580357 | rs1430707 | 2 | 131282490 | FAM123C | downstream | | |
| TT | SNP_A-8673617 | rs2615903 | 11 | 33414403 | HIPK3 | upstream | | |
| TT | SNP_A-8673617 | rs2615903 | 11 | 33414403 | HIPK3 | upstream | | |
| AA | SNP_A-8300818 | rs2274703 | 1 | 203620091 | LEMD1 | intron | | |
| AT | SNP_A-8300818 | rs2274703 | 1 | 203620091 | LEMD1 | intron | | |
| TT | SNP_A-4218063 | rs10919593 | 1 | 197077995 | PTPRC | upstream | | |
| AT | SNP_A-4218063 | rs10919593 | 1 | 197077995 | PTPRC | upstream | | |
| TT | SNP_A-8673617 | rs2615903 | 11 | 33414403 | HIPK3 | upstream | | |
| CC | SNP_A-8639378 | rs12432648 | 14 | 39997127 | LRFN5 | upstream | FBXO33 | upstream |
| CC | SNP_A-8639378 | rs12432648 | 14 | 39997127 | LRFN5 | upstream | FBXO33 | upstream |
| TT | SNP_A-4218063 | rs10919593 | 1 | 197077995 | PTPRC | upstream | | |
| AT | SNP_A-4218063 | rs10919593 | 1 | 197077995 | PTPRC | upstream | | |
| CC | SNP_A-8300818 | rs2274703 | 1 | 203620091 | LEMD1 | intron | | |

TABLE 11
Characteristics of Buffered Genotype Affecting Multiple Genes

| | | | | Physical | | | | |
|---|---|---|---|---|---|---|---|---|
| Geno- | | dbSNP- | Chr- | position- | 1st Gene- | Position 1- | 2nd Gene- | Position 2- |
| Buffered | Buffered | Buffered | Buffered | Buffered | Buffered | Buffered | Buffered | Buffered |
| AA | SNP_A-2294709 | rs3749796 | 5 | 179653982 | GFPT2 | downstream | MAPK9 | upstream |
| AA | SNP_A-2294709 | rs3749796 | 5 | 179653982 | GFPT2 | downstream | MAPK9 | upstream |
| AA | SNP_A-2294709 | rs3749796 | 5 | 179653982 | GFPT2 | downstream | MAPK9 | upstream |
| AA | SNP_A-2294709 | rs3749796 | 5 | 179653982 | GFPT2 | downstream | MAPK9 | upstream |
| AA | SNP_A-2294709 | rs3749796 | 5 | 179653982 | GFPT2 | downstream | MAPK9 | upstream |
| AA | SNP_A-2294709 | rs3749796 | 5 | 179653982 | GFPT2 | downstream | MAPK9 | upstream |
| AA | SNP_A-2294709 | rs3749796 | 5 | 179653982 | GFPT2 | downstream | MAPK9 | upstream |
| AA | SNP_A-2294709 | rs3749796 | 5 | 179653982 | GFPT2 | downstream | MAPK9 | upstream |
| AA | SNP_A-2294709 | rs3749796 | 5 | 179653982 | GFPT2 | downstream | MAPK9 | upstream |
| AA | SNP_A-2294709 | rs3749796 | 5 | 179653982 | GFPT2 | downstream | MAPK9 | upstream |
| AA | SNP_A-2294709 | rs3749796 | 5 | 179653982 | GFPT2 | downstream | MAPK9 | upstream |
| AA | SNP_A-2294709 | rs3749796 | 5 | 179653982 | GFPT2 | downstream | MAPK9 | upstream |
| AA | SNP_A-4218063 | rs10919593 | 1 | 197077995 | PTPRC | downstream | | |
| AA | SNP_A-4218063 | rs10919593 | 1 | 197077995 | PTPRC | downstream | | |
| AA | SNP_A-4218063 | rs10919593 | 1 | 197077995 | PTPRC | downstream | | |
| AA | SNP_A-4218063 | rs10919593 | 1 | 197077995 | PTPRC | downstream | | |
| AA | SNP_A-4218063 | rs10919593 | 1 | 197077995 | PTPRC | downstream | | |
| AA | SNP_A-4218063 | rs10919593 | 1 | 197077995 | PTPRC | downstream | | |
| AA | SNP_A-4282690 | rs17120428 | 1 | 60494495 | NFIA | upstream | | |
| CC | SNP_A-8300818 | rs2274703 | 1 | 203620091 | LEMD1 | intron | | |
| CC | SNP_A-8300818 | rs2274703 | 1 | 203620091 | LEMD1 | intron | | |
| CC | SNP_A-8300818 | rs2274703 | 1 | 203620091 | LEMD1 | intron | | |
| CC | SNP_A-8300818 | rs2274703 | 1 | 203620091 | LEMD1 | intron | | |
| CC | SNP_A-8300818 | rs2274703 | 1 | 203620091 | LEMD1 | intron | | |
| CC | SNP_A-8300818 | rs2274703 | 1 | 203620091 | LEMD1 | intron | | |
| CC | SNP_A-8673617 | rs2615903 | 11 | 33414403 | HIPK3 | downstream | | |
| TT | SNP_A-2294709 | rs3749796 | 5 | 179653982 | GFPT2 | downstream | MAPK9 | upstream |
| TT | SNP_A-2294709 | rs3749796 | 5 | 179653982 | GFPT2 | downstream | MAPK9 | upstream |
| TT | SNP_A-2294709 | rs3749796 | 5 | 179653982 | GFPT2 | downstream | MAPK9 | upstream |
| TT | SNP_A-2294709 | rs3749796 | 5 | 179653982 | GFPT2 | downstream | MAPK9 | upstream |
| TT | SNP_A-2294709 | rs3749796 | 5 | 179653982 | GFPT2 | downstream | MAPK9 | upstream |
| TT | SNP_A-2294709 | rs3749796 | 5 | 179653982 | GFPT2 | downstream | MAPK9 | upstream |
| TT | SNP_A-2294709 | rs3749796 | 5 | 179653982 | GFPT2 | downstream | MAPK9 | upstream |
| TT | SNP_A-2294709 | rs3749796 | 5 | 179653982 | GFPT2 | downstream | MAPK9 | upstream |
| TT | SNP_A-2294709 | rs3749796 | 5 | 179653982 | GFPT2 | downstream | MAPK9 | upstream |
| TT | SNP_A-2294709 | rs3749796 | 5 | 179653982 | GFPT2 | downstream | MAPK9 | upstream |
| TT | SNP_A-2294709 | rs3749796 | 5 | 179653982 | GFPT2 | downstream | MAPK9 | upstream |
| TT | SNP_A-2294709 | rs3749796 | 5 | 179653982 | GFPT2 | downstream | MAPK9 | upstream |
| GG | SNP_A-4218063 | rs10919593 | 1 | 197077995 | PTPRC | downstream | | |
| GG | SNP_A-4218063 | rs10919593 | 1 | 197077995 | PTPRC | downstream | | |
| GG | SNP_A-4218063 | rs10919593 | 1 | 197077995 | PTPRC | downstream | | |
| GG | SNP_A-4218063 | rs10919593 | 1 | 197077995 | PTPRC | downstream | | |
| GG | SNP_A-4218063 | rs10919593 | 1 | 197077995 | PTPRC | downstream | | |
| GG | SNP_A-4282690 | rs17120428 | 1 | 60494495 | NFIA | upstream | | |
| GG | SNP_A-4282690 | rs17120428 | 1 | 60494495 | NFIA | upstream | | |
| GG | SNP_A-4282690 | rs17120428 | 1 | 60494495 | NFIA | upstream | | |
| GG | SNP_A-4282690 | rs17120428 | 1 | 60494495 | NFIA | upstream | | |
| TT | SNP_A-8300818 | rs2274703 | 1 | 203620091 | LEMD1 | intron | | |
| TT | SNP_A-8300818 | rs2274703 | 1 | 203620091 | LEMD1 | intron | | |
| TT | SNP_A-8300818 | rs2274703 | 1 | 203620091 | LEMD1 | intron | | |
| TT | SNP_A-8300818 | rs2274703 | 1 | 203620091 | LEMD1 | intron | | |
| TT | SNP_A-8300818 | rs2274703 | 1 | 203620091 | LEMD1 | intron | | |
| TT | SNP_A-8300818 | rs2274703 | 1 | 203620091 | LEMD1 | intron | | |
| TT | SNP_A-8300818 | rs2274703 | 1 | 203620091 | LEMD1 | intron | | |
| AA | SNP_A-2294709 | rs3749796 | 5 | 179653982 | GFPT2 | downstream | MAPK9 | upstream |
| AA | SNP_A-8347387 | rs2525841 | 12 | 80054239 | ACSS3 | intron | | |
| CG | SNP_A-1881982 | rs2237342 | 7 | 27762767 | TAX1BP1 | intron | | |
| CG | SNP_A-1881982 | rs2237342 | 7 | 27762767 | TAX1BP1 | intron | | |
| CT | SNP_A-8445436 | rs642053 | 1 | 217173829 | LOC643717 | intron | | |
| CT | SNP_A-8445436 | rs642053 | 1 | 217173829 | LOC643717 | intron | | |
| AG | SNP_A-8446732 | rs17154320 | 7 | 80149730 | SEMA3C | downstream | | |
| GT | SNP_A-8456832 | rs12857956 | X | 4452855 | NLGN4X | downstream | | |
| CT | SNP_A-8682320 | rs12838423 | X | 4482942 | NLGN4X | downstream | | |
| TT | SNP_A-8445436 | rs642053 | 1 | 217173829 | LOC643717 | intron | | |
| TT | SNP_A-8445436 | rs642053 | 1 | 217173829 | LOC643717 | intron | | |
| CC | SNP_A-8475711 | rs7827228 | 8 | 2577400 | CSMD1 | downstream | MYOM2 | downstream |

### REFERENCES

1. Barzilai N, Schuldiner A R. Searching for human longevity genes: The future history of gerontology in the post-genomic era. Journal of Gerontology. 56A:M83-M87.
2. Perls T T, Bubrick E, Wagner C G, Vijg J, Kruglyak L. Siblings of centenarians live longer. Lancet. 351: 1560, 1998.
3. Puca A A, Daly M J, Brewster S J, Matise T C, Barrett J, Shea-Drinkwater M, et al. A genome-wide scan for linkage to human exceptional longevity identifies a locus on chromosome 4. Proc Natl Acad Sci USA. 98:10505-8, 2001.
4. Atzmon G, Schechter C, Greiner W, Davidson D, Rennert G, Barzilai N. Clinical Phenotype of Families with Longevity. J Am Geriat Soc 52(2):274-277, 2004.
5. Evert J, Lawler E, Perls T. Morbidity Profiles of Ventenarians: Survivors, Delayers and Escapers. J. Gerontol. A. Biol. Sci. Med. Sci. 58(3):232-7, 2003.
6. Bergman A, Atzmon G, Ye K, MacCarthy T, Barilai N. Buffering mechanisms in aging: a systems approach toward uncovering the genetic component of aging.
7. Olshansky S J, Passaro D J, Hershow R C, Layden J, Carnes B A, et al. (2005) A potential decline in life expectancy in the United States in the 21st century. N Engl J Med 352:1138-45.
8. Nissen H, Hansen A B, Guldberg P, Petersen N E, Larsen M L, et al. (1994) Detection of a single base deletion in codon 424 of the los density lipoprotein receptor gene in a Danish family with familial hypercholesterolemia. Atherosclerosis 111:209-215.
9. Deschamps O, Hondekjin J C, Van Acker P, Deslypere J P, Heller F R, et al. (1997) High prevalence of a novel mutation in the exon 4 of the low-density lipoprotein receptor gene causing familial hypercholosterolemia in Belgium. Clin Genet 51:303-308.
10. Lancaster J M, Carney M E, Futreal P A. (1997) BRCA 1 and 2—A genetic link to familial breast and ovarian cancer. medscape Womens Health 2:7.
11. Roth G S, Lane M A, Ingram D k, Mattison J A, Elahi D, et al. (2002) Biomarkers of caloric restriction may predict longevity in humans. Science 297:811.

1. A method for identifying a subject's likelihood of achieving longevity comprising detecting at least one of the following single nucleotide polymorphisms in a sample of a subject's DNA: nucleotide A at rs2410082, nucleotide A at rs11094258, nucleotide C at rs12131971, nucleotide C at rs966272, nucleotide G at rs6851158, nucleotide G at rs12556351, nucleotide G at rs5991963, nucleotide T at rs1047972, or nucleotide T at rs11001056, wherein detection of the presence of one of more of the single nucleotide polymorphisms identifies the subject as likely to achieve of longevity.

2. The method of claim 1, comprising detecting at least one of the following single nucleotide polymorphisms: nucleotide A at rs11094258, nucleotide C at rs966272, or nucleotide G at rs12556351.

3.-8. (canceled)

9. The method of claim 1, comprising detecting two or more of the single nucleotide polymorphisms.

10. The method of claim 1, further comprising obtaining the sample of the subject's DNA from the subject prior to detecting the one or more single nucleotide polymorphisms.

11. A method for identifying a subject's likelihood of developing an age-related disease comprising detecting at least one of the following single nucleotide polymorphisms in a sample of a subject's DNA: nucleotide A at rs16951040, nucleotide A at rs17625605, nucleotide A at rs3743180, nucleotide A at rs325380, nucleotide A at rs1528061, nucleotide C at rs17095079, nucleotide A at rs10775623, nucleotide A at rs10515725, nucleotide C at rs2061007, nucleotide A at rs4479454, nucleotide C at rs7630086, nucleotide C at rs3865868, nucleotide C at rs16987522, nucleotide C at rs16916036, nucleotide A at rs11818525, nucleotide A at rs17696856, nucleotide A at rs17014445, nucleotide T at rs1808723, nucleotide T at rs17049044, nucleotide G at rs11497898, nucleotide G at rs16916055, nucleotide T at rs2274703, nucleotide T at rs7067297, nucleotide G at rs2525841, nucleotide G at rs5951437, nucleotide T at rs1991921, nucleotide T at rs11247124, nucleotide T at rs3848883, or nucleotide G at rs5981402,
wherein detection of the presence of one or more of the single nucleotide polymorphisms in the subject's DNA identifies the subject as likely to develop an age-related disease.

12. The method of claim 11, comprising detecting at least one of the following single nucleotide polymorphisms: nucleotide A at rs3743180, or nucleotide T at rs1991921.

13. The method of claim 11, comprising detecting at least one of the following single nucleotide polymorphisms: nucleotide A at rs325380, nucleotide C at rs2061007, nucleotide T at rs1808723, or nucleotide T at rs11247124.

14. The method of claim 13, comprising detecting at least one of the following single nucleotide polymorphisms: nucleotide A at rs325380 or nucleotide C at rs2061007.

15. The method of claim 13, comprising detecting at least one of the following single nucleotide polymorphisms: nucleotide T at rs1808723 or nucleotide T at rs11247124.

16. The method of claim 11, comprising detecting at least one of the following single nucleotide polymorphisms: nucleotide C at rs16916036, or nucleotide G at rs16916055.

17-37. (canceled)

38. The method of claim 11, comprising detecting two or more of the single nucleotide polymorphisms.

39. The method of claim 11, wherein the age-related disease is a cardiovascular disease, diabetes mellitus, Alzheimer's disease, or a cancer.

40. The method of claim 39, wherein the age-related disease is cardiovascular disease and is hypertension, myocardial infarction, stroke, or transient ischemic attack.

41. A method for identifying a subject's likelihood of achieving longevity comprising detecting in a sample of the subject's DNA at least one of the following combinations of a genotype at a single nucleotide polymorphism from column 1 and a corresponding genotype at a single nucleotide polymorphism from column 2:

| | |
|---|---|
| Column 1 | Column 2 |
| | Genotype | dbSNP | Genotype | dbSNP |
|---|---|---|---|---|
| 1 | CG | rs10041500 | AG | rs5912192 |
| 2 | CC | rs10738555 | GG | rs10919593 |
| 3 | GC | rs10738555 | GG | rs10919593 |
| 4 | TT | rs10919593 | CT | rs642053 |
| 5 | AT | rs10919593 | CT | rs642053 |
| 6 | CT | rs11738547 | CC | rs11030746 |
| 7 | CT | rs11738547 | TT | rs11030746 |
| 8 | CT | rs11738547 | AA | rs1257689 |
| 9 | CT | rs11738547 | GG | rs17120428 |
| 10 | CT | rs11738547 | AA | rs3749796 |
| 11 | CT | rs11738547 | TT | rs3749796 |
| 12 | TT | rs12276840 | AC | rs10227794 |
| 13 | TT | rs12276840 | CC | rs10227794 |
| 14 | TT | rs12276840 | CC | rs2274703 |
| 15 | CC | rs12276840 | CC | rs2274703 |
| 1 | CC | rs12348066 | GG | rs496666 |
| 17 | CG | rs12348066 | GG | rs496666 |
| 18 | CC | rs12348066 | CC | rs536789 |
| 19 | CG | rs12348066 | CC | rs536789 |
| 20 | CC | rs12348066 | GG | rs5912192 |
| 21 | CG | rs12348066 | GG | rs5912192 |
| 22 | CC | rs12348066 | TT | rs7063827 |
| 23 | CG | rs12348066 | TT | rs7063827 |
| 24 | CC | rs12432648 | CT | rs12838423 |
| 25 | GG | rs12432648 | CT | rs12838423 |
| 26 | CC | rs12432648 | GT | rs12857956 |
| 27 | GG | rs12432648 | GT | rs12857956 |
| 28 | AG | rs1255575 | CC | rs2862274 |
| 29 | AG | rs1255575 | TT | rs2862274 |
| 30 | CC | rs1255575 | CC | rs2862274 |
| 31 | CG | rs1255575 | CC | rs2862274 |
| 32 | CC | rs1255575 | TT | rs2862274 |
| 33 | CG | rs1255575 | TT | rs2862274 |
| 34 | GG | rs1430707 | TT | rs2274703 |
| 35 | GG | rs1430707 | CC | rs2862274 |
| 36 | GG | rs1430707 | TT | rs2862274 |
| 37 | GG | rs1430707 | AA | rs3749796 |
| 38 | GG | rs1461809 | TT | rs3816612 |
| 39 | CC | rs1499953 | AA | rs10919593 |
| 40 | AG | rs1528708 | GG | rs4595891 |
| 41 | TT | rs16880188 | CC | rs11030746 |
| 42 | AT | rs16880188 | CC | rs11030746 |
| 43 | TT | rs16880188 | TT | rs11030746 |
| 44 | AT | rs16880188 | TT | rs11030746 |
| 45 | TT | rs16880188 | CC | rs17003277 |
| 46 | AT | rs16880188 | CC | rs17003277 |
| 47 | TT | rs16880188 | TT | rs17003277 |
| 48 | AT | rs16880188 | TT | rs17003277 |
| 49 | TT | rs16951040 | CC | rs1324362 |
| 50 | AT | rs16951040 | CC | rs1324362 |
| 51 | CC | rs16951040 | AG | rs17154320 |
| 52 | TT | rs17014445 | GG | rs10919593 |
| 53 | AT | rs17014445 | GG | rs10919593 |
| 54 | TT | rs17014445 | GG | rs10919593 |
| 55 | GG | rs17014445 | AA | rs10919593 |
| 56 | GG | rs17014445 | GG | rs10919593 |
| 57 | GG | rs17014445 | AA | rs10919593 |
| 58 | TT | rs17014445 | CC | rs11103184 |
| 59 | AT | rs17014445 | CC | rs11103184 |
| 60 | GG | rs17014445 | CC | rs11103184 |
| 61 | TT | rs17014445 | CC | rs11103512 |
| 62 | AT | rs17014445 | CC | rs11103512 |
| 63 | GG | rs17014445 | CC | rs11103512 |
| 64 | TT | rs17014445 | CC | rs11183915 |
| 65 | AT | rs17014445 | CC | rs11183915 |
| 66 | GG | rs17014445 | CC | rs11183915 |
| 67 | GG | rs17014445 | AG | rs17154320 |
| 68 | TT | rs17014445 | TT | rs1822910 |
| 69 | AT | rs17014445 | TT | rs1822910 |
| 70 | GG | rs17014445 | TT | rs1822910 |
| 71 | GG | rs17014445 | AA | rs2525841 |
| 72 | GG | rs17014445 | AA | rs3749796 |
| 73 | GG | rs17014445 | AA | rs3925518 |
| 74 | GG | rs17014445 | TT | rs6077004 |
| 75 | GG | rs17014445 | CT | rs6419449 |
| 76 | GG | rs17014445 | CG | rs6540375 |
| 77 | GG | rs17014445 | AT | rs6641231 |
| 78 | GG | rs17014445 | CC | rs7827228 |
| 79 | TT | rs17014445 | CT | rs9586459 |
| 80 | AT | rs17014445 | CT | rs9586459 |
| 81 | TT | rs17014445 | TT | rs9586459 |
| 82 | AT | rs17014445 | TT | rs9586459 |
| 83 | GG | rs17014445 | CT | rs9586459 |
| 84 | GG | rs17014445 | TT | rs9586459 |
| 85 | AA | rs17049044 | CT | rs3859965 |
| 86 | AT | rs17049044 | CT | rs3859965 |
| 87 | CC | rs17049044 | AA | rs553369 |
| 88 | AA | rs17083201 | GT | rs2144497 |
| 89 | AT | rs17083201 | GT | rs2144497 |
| 90 | AA | rs17154320 | CC | rs10278652 |
| 91 | GG | rs17154320 | CC | rs10278652 |
| 92 | AA | rs17372695 | CC | rs2862274 |
| 93 | AT | rs17372695 | CC | rs2862274 |
| 94 | AA | rs17372695 | TT | rs2862274 |
| 95 | AT | rs17372695 | TT | rs2862274 |
| 96 | CT | rs17775764 | AG | rs5912192 |
| 97 | TT | rs1822910 | AC | rs11625236 |
| 98 | TT | rs1822910 | CC | rs2274703 |
| 99 | TT | rs1822910 | CT | rs2613216 |
| 100 | TT | rs1822910 | TT | rs2613216 |
| 101 | TT | rs1822910 | AA | rs2623149 |
| 102 | TT | rs1822910 | AG | rs2623149 |
| 103 | TT | rs1822910 | GG | rs5981167 |
| 104 | AT | rs1822910 | GG | rs5981167 |
| 105 | AA | rs1991921 | CC | rs11030746 |
| 106 | AT | rs1991921 | CC | rs11030746 |
| 107 | AA | rs1991921 | TT | rs11030746 |
| 108 | AT | rs1991921 | TT | rs11030746 |
| 109 | AA | rs1991921 | TT | rs3749796 |
| 110 | AT | rs1991921 | TT | rs3749796 |
| 111 | AA | rs1991921 | CC | rs3816612 |
| 112 | AT | rs1991921 | CC | rs3816612 |
| 113 | GG | rs2012129 | CC | rs11030746 |
| 114 | CG | rs2012129 | CC | rs11030746 |
| 115 | GG | rs2012129 | TT | rs11030746 |
| 116 | CG | rs2012129 | TT | rs11030746 |
| 117 | GG | rs2012129 | TT | rs3749796 |
| 118 | CG | rs2012129 | TT | rs3749796 |
| 119 | GG | rs2061007 | CC | rs11030746 |
| 120 | CG | rs2061007 | CC | rs11030746 |
| 121 | GG | rs2061007 | TT | rs11030746 |
| 122 | CG | rs2061007 | TT | rs11030746 |
| 123 | GG | rs2061007 | AA | rs16902320 |
| 124 | CG | rs2061007 | AA | rs16902320 |
| 125 | GG | rs2061007 | CC | rs16902320 |
| 126 | CG | rs2061007 | CC | rs16902320 |
| 127 | GG | rs2061007 | CC | rs17003277 |
| 128 | CG | rs2061007 | CC | rs17003277 |
| 129 | GG | rs2061007 | TT | rs1822910 |
| 130 | CG | rs2061007 | TT | rs1822910 |
| 131 | GG | rs2061007 | TT | rs2274703 |
| 132 | CG | rs2061007 | TT | rs2274703 |
| 133 | GG | rs2061007 | CC | rs2862274 |
| 134 | CG | rs2061007 | CC | rs2862274 |
| 135 | GG | rs2061007 | TT | rs2862274 |
| 136 | CG | rs2061007 | TT | rs2862274 |
| 137 | GG | rs2061007 | TT | rs3749796 |
| 138 | CG | rs2061007 | TT | rs3749796 |
| 139 | GG | rs2061007 | CC | rs3816612 |
| 140 | CG | rs2061007 | CC | rs3816612 |
| 141 | GG | rs2061007 | TT | rs3816612 |
| 142 | CG | rs2061007 | TT | rs3816612 |
| 143 | GG | rs2098846 | CC | rs17003277 |
| 144 | CG | rs2098846 | CC | rs17003277 |
| 145 | GG | rs2098846 | TT | rs17003277 |
| 146 | CG | rs2098846 | TT | rs17003277 |
| 147 | GG | rs2098846 | TT | rs2274703 |
| 148 | CG | rs2098846 | TT | rs2274703 |
| 149 | CC | rs2274703 | CG | rs2237342 |
| 150 | AA | rs2274703 | CG | rs2237342 |
| 151 | AT | rs2274703 | CG | rs2237342 |
| 152 | CC | rs2274703 | CC | rs7827228 |
| 153 | AG | rs2382993 | CC | rs11103184 |
| 154 | AA | rs2525841 | CT | rs12838423 |
| 155 | AA | rs2525841 | GT | rs12857956 |
| 156 | CC | rs2525841 | AG | rs5981167 |
| 157 | CG | rs2525841 | AG | rs5981167 |
| 158 | CC | rs2525841 | GG | rs5981167 |
| 159 | CG | rs2525841 | GG | rs5981167 |
| 160 | TT | rs2615903 | AG | rs17154320 |
| 161 | TT | rs2615903 | AA | rs2525841 |
| 162 | TT | rs2615903 | AA | rs3749796 |
| 163 | AA | rs2762933 | CC | rs2862274 |
| 164 | AT | rs2762933 | CC | rs2862274 |
| 165 | AA | rs2762933 | TT | rs2862274 |
| 166 | AT | rs2762933 | TT | rs2862274 |
| 167 | TT | rs325380 | CC | rs11030746 |
| 168 | AT | rs325380 | CC | rs11030746 |
| 169 | TT | rs325380 | TT | rs11030746 |
| 170 | AT | rs325380 | TT | rs11030746 |
| 171 | TT | rs325380 | TT | rs3749796 |
| 172 | AT | rs325380 | TT | rs3749796 |
| 173 | CC | rs368866 | CC | rs17003277 |
| 174 | CG | rs368866 | CC | rs17003277 |
| 175 | CC | rs368866 | TT | rs17003277 |
| 176 | CG | rs368866 | TT | rs17003277 |
| 177 | TT | rs4686457 | AA | rs10919593 |
| 178 | TT | rs4686457 | TT | rs11030746 |
| 179 | TT | rs4686457 | AA | rs16902320 |
| 180 | TT | rs4686457 | CC | rs16902320 |
| 181 | TT | rs4686457 | TT | rs2862274 |
| 182 | TT | rs4686457 | CC | rs2862274 |
| 183 | TT | rs4686457 | TT | rs3749796 |
| 184 | AA | rs4686870 | AA | rs10919593 |
| 185 | AA | rs4686870 | CC | rs16902320 |
| 186 | AA | rs4686870 | CC | rs2862274 |
| 187 | AA | rs4686870 | TT | rs2862274 |
| 188 | AA | rs4686870 | AA | rs3749796 |
| 189 | AA | rs4686870 | TT | rs3749796 |
| 190 | AA | rs533391 | CG | rs2237342 |
| 191 | AT | rs533391 | CG | rs2237342 |
| 192 | CT | rs5979604 | AA | rs3749796 |
| 193 | GG | rs6066159 | CC | rs10278652 |
| 194 | CG | rs6066159 | CC | rs10278652 |
| 195 | GG | rs6066159 | AA | rs10919593 |
| 196 | CG | rs6066159 | AA | rs10919593 |
| 197 | GG | rs6066159 | AA | rs10920481 |
| 198 | CG | rs6066159 | AA | rs10920481 |
| 199 | TT | rs6094553 | CC | rs10278652 |
| 200 | AT | rs6094553 | CC | rs10278652 |
| 201 | TT | rs6851158 | GG | rs17120428 |
| 202 | AT | rs6851158 | GG | rs17120428 |
| 203 | AA | rs696405 | TT | rs2862274 |
| 204 | AA | rs7106605 | AA | rs3749796 |
| 205 | GG | rs7106605 | AA | rs3749796 |
| 206 | AA | rs7130910 | AA | rs3749796 |
| 207 | GG | rs7130910 | AA | rs3749796 |
| 208 | CC | rs7153013 | TT | rs17003277 |
| 209 | TT | rs7153013 | TT | rs17003277 |
| 210 | GG | rs7221263 | CC | rs11030746 |
| 211 | GG | rs7221263 | TT | rs11030746 |
| 212 | GG | rs7221263 | CC | rs11103184 |
| 213 | GG | rs7221263 | TT | rs11103184 |
| 214 | GG | rs7221263 | AA | rs16902320 |
| 215 | GG | rs7221263 | CC | rs17003277 |
| 216 | GG | rs7221263 | TT | rs17003277 |
| 217 | GG | rs7221263 | CC | rs17078925 |
| 218 | GG | rs7221263 | TT | rs17078925 |
| 219 | GG | rs7221263 | AA | rs17120428 |
| 220 | GG | rs7221263 | GG | rs17120428 |
| 221 | GG | rs7221263 | CC | rs2274703 |
| 222 | GG | rs7221263 | TT | rs2274703 |
| 223 | GG | rs7221263 | CC | rs2615903 |
| 224 | GG | rs7221263 | CC | rs2862274 |
| 225 | GG | rs7221263 | TT | rs2862274 |
| 226 | GG | rs7221263 | AA | rs3749796 |
| 227 | GG | rs7221263 | TT | rs3749796 |
| 228 | GG | rs7221263 | CC | rs3816612 |
| 229 | GG | rs7221263 | TT | rs3816612 |
| 230 | GG | rs7221263 | GG | rs4595891 |
| 231 | GG | rs7221263 | TT | rs4595891 |
| 232 | GG | rs7579939 | CC | rs2274703 |
| 233 | GG | rs7579939 | AA | rs3749796 |
| 234 | CC | rs999430 | CC | rs2274703 |
| 235 | CC | rs999430 | TT | rs2274703 |

wherein the presence of one or more of the combinations of the recited genotype at the single nucleotide polymorphism from column 1 and the corresponding recited genotype at the single nucleotide polymorphism from column 2 indicates that the subject is likely to develop an age-related disease.

42.-47. (canceled)

48. The method of claim 1, wherein the subject is a human.
